(19) United States
(12) Patent Application Publication
(10) Pub. No.: US 2010/0022637 A1
(43) Pub. Date: Jan. 28, 2010

(54) IDENTIFICATION OF ANTI-CANCER COMPOUNDS AND COMPOUNDS FOR TREATING HUNTINGTON'S DISEASE AND METHODS OF TREATMENT THEREOF

(75) Inventors: Brent R. Stockwell, New York, NY (US); Inese Smukste, New York, NY (US)

Correspondence Address: BRYAN CAVE LLP, 1290 AVENUE OF THE AMERICAS, NEW YORK, NY, 10104, US

(73) Assignee: THE TRUSTEES OF COLUMBIA UNIVERSITY IN THE CITY OF NEW YORK, New York, NY (US)

(21) Appl. No.: 11/992,867

(22) PCT Filed: Sep. 29, 2006
(86) PCT No.: PCT/US2006/038132

§ 371 Date: Sep. 4, 2009

Related U.S. Application Data
(60) Provisional application No. 60/721,667, filed on Sep. 29, 2005.
(60) Provisional application No. 60/771,187, filed on Feb. 7, 2006.

(51) Int. Cl. A61K 31/35; C12N 5/02; G01N 33/00; A61K 31/16; C07D 315/00; A61P 35/04; A61P 25/28

(52) U.S. Cl. 514/459; 435/375; 436/86; 514/613; 549/419

(57) ABSTRACT

Small molecule screening via high-throughput screening (HTS) methods was employed to identify compounds useful for treating or preventing cancer (such as compounds that enable cells to overcome E6-oncoprotein-mediated drug resistance) or neurodegenerative disorders (such as Huntington's disease, HD). Compounds were identified that potentiate the lethality of anti-tumor agents as well as rescue a disease-state lethality. These compounds are acylated secondary amines referred to herein as indoxins and revertins.

The work described herein was supported in whole, or in part, by National Cancer Institute grant R01CA97061. The United States Government may have certain rights to the invention.

### BACKGROUND OF THE INVENTION

The ability of tumor cells to survive after treatment with any of numerous unrelated anti-cancer drugs is known as multidrug resistance (MDR). Specifically, resistance to apoptosis-inducing drugs is one of the hallmarks of cancer: most cancer cells eventually acquire alterations that enable them to evade apoptosis. This ability contributes to the drug-resistant phenotype found in human cancers. For example, the tumor-suppressor protein p53, which is a central signaling protein involved in apoptosis, is mutated in more than 50% of human cancers. Most commonly, the TP53 gene is inactivated via mutation. However, amplification of the Murine Double Minute 2 (MDM2) oncogene, which encodes an E3 ubiquitin ligase that targets p53 for proteasomal degradation, also leads to loss of p53 protein. In addition, viral oncogenes, such as HPV E6, SV40 large T antigen and human hepatitis B virus X protein, are capable of interfering with p53 function and thereby increasing the resistance of tumor cells to chemotherapeutic agents.

HPV type 16 and 18 (HPV16 and HPV18) are the major causative factors in cervical cancer. More than 90% of cervical cancer patients have been infected with these high-risk viruses that are capable of immortalizing and transforming normal human cells. HPV16 and HPV18 are associated with high-grade squamous intra-epithelial lesions, invasive cervical carcinomas, anal, peri-anal, vulvar and penile cancers. Two viral genes, E6 and E7, are required for HPV16/18-mediated carcinogenesis.

The viral protein E6 induces p53 ubiquitination and degradation by complexing with E6 Associated Protein (E6AP), which is an E3 ligase. E6/E6AP-mediated degradation of p53 allows tumor cells to overcome cell-cycle checkpoint control in DNA-damaged cells, contributing to the mutagenic and anti-apoptotic effects of E6. E6 also facilitates degradation of the pro-apoptotic protein BCL2-antagonist/killer (BAK), activates telomerase and prevents degradation of SRC-family kinases.

HPV-induced malignant growth of cervical cancer depends upon continuous expression of E6. Although there are no specific therapies available, various approaches have been used in attempts to overcome E6-induced resistance to apoptosis. Antisense inhibition of the E6 oncogene blocks the malignant phenotype of cervical cancer cells and siRNAs targeting of the viral E6 oncogene mRNA effectively kill HPV-positive cancer cells. HPV16 E6 protein-binding aptamers have been shown to eliminate HPV16-positive cancer cells by inducing apoptosis.

Small molecules are more easily adaptable for therapeutic use than peptide or nucleic acid reagents, but few examples of small molecules that overcome E6-induced drug resistance have been described in the literature. The glycolytic pathway inhibitor 2-deoxyglucose (2-DG) has been reported to suppress transcription of HPV18 in cervical carcinoma cells. The antioxidant pyrrolidine-dithiocarbamate (PDTC) suppresses HPV16 expression in human keratinocytes by modulating activity of the transcription factor AP-1. Dithiobisamine-based substances bind to the HPV16 E6 zinc finger and inactivate its activity. The histone deacetylase (HDAC) inhibitors sodium butyrate and trichostatin A induce G1/S phase arrest in HPV18-positive cervical carcinoma cells, followed by apoptosis, circumventing E6 anti-apoptotic activity. These examples suggest that it is possible to find small molecules that suppress HPV-induced tumorigenicity, although the reported examples lack specificity.

Numerous efforts to develop HD therapies have been initiated but currently there is no therapy for this fatal disease. Huntington's disease is an autosomal dominant neurodegenerative disease that involves neuronal loss in the striatum and cortex. This neuronal degeneration results in progressive motor and behavioral abnormalities that are ultimately fatal. The disease is caused by polyQ expansion (>36Q) in the amino-terminus of the htt protein. Both a loss of function of normal length polyQ (wild type (WT)) containing htt and a gain of function of expanded polyQ (mutant) containing htt protein have been implicated in HD. WT htt is essential for embryonic development in mice, prevents neuronal cell death in some models and has been implicated in diverse cellular functions. A variety of molecular mechanisms have been proposed to explain mutant htt toxicity, including protein aggregation, altered transcriptional regulation, mitochondrial dysfunction, defects in intracellular transport and activation of apoptotic machinery. The contributions of these mechanisms to neuronal pathology are unclear.

### SUMMARY OF THE INVENTION

Described herein is a high-throughput screening method useful in identifying agents for treating or preventing diseases or conditions, such as cancer, the presence or development of tumors, or other conditions characterized by excessive cell proliferation, or neurodegenerative disorders. An agent identified by such a screening method can be used to treat or prevent cancer in a subject, such as a human in need of treatment or prevention. Also described herein is a genotype-selective method for identifying agents that can be used to prevent or treat the neurodegenerative disease, Huntington's Disease (HD).

In one embodiment, the invention relates to a class of secondary acylated amines, referred to herein as indoxins. In another embodiment, the invention relates to analogs of indoxins that selectively kill or inhibit the growth of (are toxic to) engineered tumorigenic cells (for example, human RKO-E6 cells). In a further embodiment of the invention, the compound of the invention is formulated with a pharmaceutically acceptable carrier.

Methods of identifying cellular components involved in tumorigenesis are disclosed in this invention. Cellular components can include, lipids, proteins, and nucleic acids. In one embodiment of the invention, the invention relates to a method of identifying a cellular component that interacts with an indoxin wherein (a) a cell, such as an engineered human tumorigenic cell, is contacted with an indoxin; and (b) a cellular component that interacts with an indoxin, either directly or indirectly, is identified. The cellular component that is identified by the method of the invention is a cellular component that interacts with an indoxin.

The present invention relates to methods of treating cancer. In one embodiment, the invention relates to a method of treating a subject having a multi-drug resistant cancer where a therapeutically effective amount of a pharmaceutically acceptable compound is administered to a subject in need of cancer treatment. In another embodiment, the cancer is characterized by engineered cells whereby the E6 oncoprotein is activated.

The invention described herein also relates to methods of treating a neurodegenerative disorder, such as Huntington's Disease. In one embodiment, the present invention discloses a method of treating a neurodegenerative disorder associated with polyglutamine (polyQ) expansion in a subject comprising administering a therapeutically effective amount of a pharmaceutically suitable compound, wherein the compound is a a class of secondary acylated amines referred to herein as revertins. In another embodiment, the revertin is revertin-20 (TABLE 3). In further embodiments, the neurodegenerative disorder is Huntington's disease.

### BRIEF DESCRIPTION OF THE DRAWINGS

FIG. 1 shows the differential effect of doxorubicin treatment on RKO and RKO-E6 cells. FIG. 1A are microscopy images of a cell viability assay of RKO and RKO-E6 cells that were treated with doxorubicin for 16 h to evaluate RKO-E6 resistance to doxorubicin. The largest differential in cell survival was observed at 0.50 μg/ml of doxorubicin. The high-throughput screen was performed in the presence of 0.50 μg/ml of doxorubicin. Compounds that restored doxorubicin-induced killing were further evaluated. FIG. 1B is a western blot of p53. p53 cellular concentration is lower in the presence of E6 (see RKO and RKO-E6). Treatment with 0.5 μg/mL doxorubicin or camptothecin for 16 h caused p53 to accumulate; in RKO-E6 cells, p53 accumulates to a lesser extent, due to E6/E6AP mediated degradation. The blot was simultaneously probed with an antibody directed against eIF-4E to control for protein loading.

FIG. 2 depicts quaternary ammonium compounds that enhance doxorubicin lethality. FIG. 2A represents the structures of quaternary ammonium compounds (QACs) that selectively upregulated doxorubicin's lethality in RKO-E6 cells. GMS-041F is an analog of benzalkonium chloride that has been reported to inhibit proliferation of several human cancer cell lines (Gastaud 1998). FIG. 2B is a graph of a cell viability assay. RKO-E6 cells were treated with cetrimonium bromide in the presence of 0.5 μg/mL doxorubicin or camptothecin for 24 h in 384-well plates. Percent inhibition of cell proliferation, measured with Alamar Blue, is shown in the graph. The 20% growth inhibition at 0 μg/ml cetrimonium bromide represent the toxicity of 0.5 μg/ml doxorubicin alone or 0.5 μg/ml camptothecin alone in RKO-E6 cells.

FIG. 3 shows protein synthesis inhibitors enhance doxorubicin lethality. FIG. 3A represents chemical structures of protein synthesis inhibitors that were found to enhance doxorubicin lethality. FIG. 3B is a cell viability graph showing the effect of cycloheximide on RKO-E6 cells in the presence of 0.5 μg/mL doxorubicin or 0.2 μg/mL podophyllotoxin. RKO-E6 cells were treated in 384-well plates for 24 h. Percent inhibition of cell proliferation, measured with Alamar Blue, is shown in the graph.

FIG. 4 depicts 11-deoxyprostaglandin E1 analogs that enhance doxorubicin lethality. RKO cells were treated with 8 μg/ml of each 11-deoxyprostaglandin E1 analog, and RKO-E6 cells were treated with 8 μg/ml of 11-deoxyprostaglandin E1 analog and 0.5 μg/ml of doxorubicin. The percent inhibition of cell proliferation is shown and listed next to each analog. Amide functionalities are colored based on their activity: active and selective analogs—red; moderately active and selective analogs—green; toxic analogs—black; and inactive analogs—blue.

FIG. 5 shows T55D7 and related analogs that enhance doxorubicin lethality. FIG. 5A are cell viability graphs. RKO-E6 cells were treated with T55D7 and in its analogs in the presence or absence of 0.5 μg/mL doxorubicin for 24 h. Percent inhibition of cell proliferation, measured with Alamar Blue, is shown in the graph. FIG. 5B represents graphs of the cell cycle distribution, as determined using flow cytometry. T55D7 induces S-phase arrest in RKO-E6 cells, but not in RKO cells. RKO and RKO-E6 cells were treated with 4 μg/ml T55D7 alone or with 0.5 μg/mL doxorubicin for 24 h and stained with propidium iodide. FIG. 5C depicts a combination dose matrix, showing the combined effect of T55D7 and doxorubicin, in RKO-E6 cells. The experimentally measured cell growth inhibition (with the Alamar Blue assay) is shown for each concentration. The calculated excess inhibition over the predicted Bliss Independence model indicates the synergy between Indoxin B and doxorubicin treatment. The predicted Bliss Independence effect was subtracted from the experimentally measured cell growth inhibition at each pair of concentrations. The color of the squares indicates the level of activity in excess of that predicted by Bliss Independence.

FIG. 6 shows that Indoxins enhance doxorubicin lethality. FIG. 6A depicts the chemical structures of Indoxin A and Indoxin B. FIG. 6B is a graph of a cell viability assay. Indoxin-B-treated RKO and RKO-E6 cells in the presence of 0.5 μg/mL doxorubicin or 0.2 μg/mL podophyllotoxin. RKO and RKO-E6 cells were treated in 384-well plates for 24 h. Percent inhibition of cell proliferation, measured with Alamar Blue, is shown in the graph. FIG. 6C is a western blot. RKO-E6 cells were treated with 0.5 μ/mL doxorubicin, 4 μg/mL indoxin A, or 4 μg/mL indoxin B for 24 h. The level of topoisomerase IIα was determined by western blot, and the membrane was re-probed for eIF4E as a loading control. FIG. 6D represents graphs of the cell cycle distribution, as determined using flow cytometry. RKO, RKO-E6, or HeLa cells were treated with 4 μg/ml of indoxin A alone or with 0.5 μg/mL doxorubicin for 24 h. Cells were stained with propidium iodide and subjected to flow cytometry. FIG. 6E is a combination dose matrix, showing the combined effect of indoxin B and doxorubicin in RKO-E6 cells. The experimentally measured cell growth inhibition (Alamar Blue assay) is shown for each concentration. The calculated excess inhibition over the predicted Bliss Independence model indicates the synergy between indoxin B and doxorubicin treatment. The predicted Bliss independence effect was subtracted from the experimentally measured cell growth inhibition at each pair of concentrations. The color of the squares indicates the level of the synergy.

FIG. 7 depicts Indoxin affinity probes used in experiments. FIG. 7A represents the structures of indoxin-biotin labels. FIG. 7B shows the structures of indoxin-benzophenone-biotin labels. FIG. 7C illustrates the structures of indoxin-benzophenone-fluorescein labels. FIG. 7D is an SDS-PAGE gel of proteins isolated using indoxin and control probes; nonspecific crosslinking was observed using the benzophenone-biotin control probe. Replacement of biotin with fluorescein as affinity tag reduced nonspecific crosslinking. FIG. 7E shows proteins were selectively isolated using the indoxin-benzophenone-fluorescein probe in-pull-down experiments, but not the control benzophenone-fluorescein probe. In a second experiment, MYO1C and ARP2 were again identified.

FIG. 8 is a schematic of the synthesis of indoxin affinity probes. Abbreviations used in the schematic of the synthesis of indoxin probes are as follows: Boc,—t-butoxycarbonyl; CDI,—N,N′-carbonyldiimidazole; DIPEA,—diisopropylethylamine; DMF,—dimethylformamide; DMSO,—dimethyl sulfoxide; Fmoc,—9-fluorenylmethoxycarbonyl; HBTU,—O-benzotriazol-1-yl-N,N,N′,N′-tetramethyluronium hexafluorephosphate; PFP,—pentafluorophenol; TFA,—trifluoroacetic acid; TFP,—tetrafluorophenol.

FIG. 9A is the chemical structure of T86N7. FIG. 9B is a graph of a cell viability assay. T86N7 showed ˜20% differential between cell growth inhibition in RKO cells and in RKO-E6 cell in the presence of 0.5 μg/ml of doxorubicin. FIG. 9C is a western blot showing that T86N7 upregulated p53 levels in RKO-E6 cells in the presence of doxorubicin. FIG. 9D is a graph of cell viability. T86N7 showed no selectivity in a number of other cancer cell lines: BJELR, A673, HeLa and TC32 cells.

FIGS. 10A-B depict graphs of cell viability. Selected compounds from the 88 hits identified in the secondary screen were tested alone (NT) in RKO and RKO-E6 cells or in the presence of doxorubicin (0.5 μg/ml for RKO6 cells and 0.25 [2g/ml for RKO cells) or 0.2 μg/ml of podophyllotoxin (PTX). The percent inhibition of cell growth, measured with Alamar Blue, is shown after treatment for 24 h. Some spatial patterning in these 384-well plates was observed. Such “plate effects” are not corrected in these data.

FIG. 11 depicts graphs of cell viability experiments as well as the structures of Indoxin analogs. A series of Indoxin B analogs and Indoxin-A-biotin were synthesized and their activity tested in RKO-E6 cells in the presence and absence of 0.5 μg/mL doxorubicin. The activity of these analogs in the cell viability assay, measured with Alamar Blue, is shown.

FIG. 12 illustrates combination dose matrices, showing the combined effect of T55D7 and doxorubicin in RKO-E6, RKO, HeLa and TC32 cells. The experimentally measured cell growth inhibition (Alamar Blue assay) is shown for each concentration. The calculated excess inhibition over predicted Bliss Independence (Keith et al., 2005) indicates the synergy between T55D7 and doxorubicin treatment. The predicted Bliss Independence effect was subtracted from the experimentally measured cell growth inhibition at each pair of concentrations. The color of the squares indicates the level of the synergy.

FIGS. 13A-B representS combination dose matrices, showing the combined effect of Indoxin A or Indoxin B and doxorubicin in RKO-E6, RKO, HeLa and TC32 cells. The experimentally measured cell growth inhibition (Alamar Blue assay) is shown for each concentration. The calculated excess inhibition over the predicted Bliss Independence model indicates the synergy between indoxins and doxorubicin treatment. The predicted Bliss Independence effect was subtracted from the experimentally measured cell growth inhibition at each pair of concentrations. The color of the squares indicates the level of the synergy.

FIG. 14 illustrates the optimization of a striatal neuronal HD assay for screening. FIG. 14A is a graph depicting fluorescence intensity in relation to increasing the cell number plated in 6 replicates, in a 384-well plate. After 6 h, cell fluorescence was determined by the calcein AM assay and is shown as average±one S.D. FIG. 14B is a graph showing the relation of coefficient of variation (CV) to cell seeding density. CV at increasing cell densities using 6 replicates was determined. FIG. 14C is a time course of calcein fluorescence signal. 1500 cells were plated/well and subjected to calcein AM assay. Fluorescence was measured over 5 h. FIG. 14D is a western blot demonstrating that T-ag protein decreases at 39° C. ST14A and N548 mutant cells were incubated at 33° C. or 39° C. for 6 h. FIG. 14E is a graph of the effect of serum concentration on cell viability. Low serum concentration decreases viability of N548 mutant cells. 1500 cells/well were plated in 384-well plates with medium containing a range of serum concentration (0-5% IFS). Cell viability was assayed after 3 d incubation at 33° C. (open squares) or 39° C. (diamonds). Error bars indicate one S.D. FIG. 14F is a bar graph of cell viability. Relative protection from cell death in WT N548 cells was compared to N548 mutant cells. 1500 cells were incubated at 39° C. for 3 d in 0.5% IFS and viability was assayed by calcein assay.

FIG. 15 represents a schematic of HTS and hit identification. FIG. 15A is a flowchart of the hit discovery process. FIG. 15B is a plot of screening data for the NINDS library compounds. 1040 compounds arrayed in 384-well plates and one DMSO plate were assayed in triplicate. One plate was assayed on the day of cell seeding as a control for complete rescue (triangles). A 50% increase in signal above the median plate signal was set as a threshold to identify hits (horizontal bar). A hit that enhances signal in triplicate wells is circled.

FIG. 16 represents detection of mutant htt non-selective and selective compounds in striatal cell lines. FIG. 16A-B are graphs depicting that pan-caspase inhibitor BOC-D-fmk inhibits caspase activity and prevents cell death non-selectively. In FIG. 16A, activation of individual caspases was monitored fluorometrically in 3 cell lines (ST14A, N548 mutant and N548 WT) under serum deprivation at 39° C. with or without BOC-D-fmk (50 μM) (ST14A and N548 mutant). Fluorescence in each sample was normalized to protein and represented relative to the fluorescence in WT N548 cells. The results are the average±SD of one experiment performed in triplicate. FIG. 16B is a dilution series of BOC-D-fmk that was tested in ST14A and N548 mutant cells where cell death was induced by 0.5% IFS at 39° C. The fluorescence values in BOC-D-fmk treated cells are expressed relative to vehicle (DMSO) treated cells. The results are the average±S.D. of an experiment performed in triplicate. FIG. 16C-H illustrates structures and dose responses of cell death rescue in a panel of different length mutant htt expressing cell lines for selective compounds: FIG. 16C-D corresponds to the N548 mutant selective compound, revertin-6; FIG. 16E-F corresponds to the N63 and N548 mutant selective compound, revertin-10; and FIG. 16G-H correspond to the N548 and FL-mutant selective compound, revertin-14.

FIG. 17 shows the identification of microtubule inhibitors based on selectivity profiling. FIG. 17A depicts the structures of compound revertin-22 and revertin-23. FIG. 17B is a table illustrating the selectivity profiles for cell death rescue by microtubule inhibitor (MTI), colchicine, and revertin-22. FIG. 17C are microscopy images of N548 mutant cells. Revertin-22 and colchicine depolymerize microtubules in N548 mutant cells. Micrographs are of β-tubulin immunofluorescence in N548 mutant cells treated with DMSO (0.1%), colchicine (400 nM) or revertin-22 (4 μg/ml) for 8 h.

FIG. 18 represents compounds that enhance neuronal survival in PC12 and C. elegans HD models. FIG. 18A are structures of two compounds revertin-1 and revertin-2. FIG. 18B is a graph representing the rescue of PC12 HD toxicity by revertin-1c (100 μg/ml) and revertin-2 (10 μg/ml). Rescue was expressed relative to BOC-D-fmk (50 μM) that was a 100% rescue. The results are the average±S.D. of two experiments performed in triplicate. FIG. 18C is a graphic representation of a survival assay. Revertin-2 (0.8 mg/ml) and revertin-1a (1 mg/ml) enhance ASH neuronal cell survival in a C. elegans HD model. ASH neuronal cell survival was assayed at 2 d after compound treatment. The results are the average±SD of three independent experiments (n=50 animals, 100 neurons). The rescue was significant * (two tail t-test, p<0.02). FIG. 18D shows that Rev-1c selectively rescues cell death in three mutant htt expressing cell lines, N63, N548, and FL mutant but not in parental ST14A cells.

FIG. 19 is a food clearance assay to determine effective drug concentrations in worms. 20 L1 synchronized N2 worms were seeded in triplicate with food suspension mixed with drug in a 96 well plate. Drug effects were assayed by monitoring the rate of food clearance (absorbance O.D. 600 nm) in revertin-2 (0.8 mg/ml), vehicle (DMSO 1.7%) and paraquat (6 mM), treated worms. The results are the average of an experiment performed in triplicate. Paraquat was used as a control as it is known to be toxic at the concentration tested (Vanfleteren J R, et al, Biochem J 1993, 292: 605-8.).

FIG. 20 is a non-limiting schematic of an embodiment to screen compounds. The labeled compound (for example, a fluorescein-conjugated indoxin) is added to the target protein-coated wells, wherein the target protein-coated wells bound with the labeled-compound can be exposed to a library of compounds. Binding of the new, unlabeled compound can displace the labeled compound and differences in fluorescence intensity is detected using colorimetric or fluorescence assays. For example, displacement of a labeled compound, such as a fluorescein-conjugated indoxin, would result in decreased fluorescence intensity.

FIG. 21A-B depicts chemical structures of active and selective analogs, wherein RKO-E6 represents the percent cell growth inhibition in RKO-E6 cells in the presence of doxorubicin and RKO is the percent cell growth inhibition in RKO cells.

FIG. 22A-B depict chemical structures of inactive analogs, wherein RKO-E6 represents the percent cell growth inhibition in RKO-E6 cells in the presence of doxorubicin and RKO is the percent cell growth inhibition in RKO cells.

FIG. 23 are chemical structures of toxic analogs, wherein RKO-E6 represents the percent cell growth inhibition in RKO-E6 cells in the presence of doxorubicin and RKO is the percent cell growth inhibition in RKO cells.

FIG. 24A-B depict chemical structures of functional groups (in blue) that inactivate analogs, wherein RKO-E6 represents the percent cell growth inhibition in RKO-E6 cells in the presence of doxorubicin and RKO is the percent cell growth inhibition in RKO cells.

FIG. 25A-C are graphs demonstrating the percent inhibition of cell growth of RKO-E6 cells treated with either a topoisomerase I or topoisomerase II inhibitor alone or co-administrated with indoxin A.

### DETAILED DESCRIPTION OF THE INVENTION

The scientific and patent literature referred to herein establishes knowledge that is available to those skilled in the art. The issued patents, patent applications, and other publications that are cited herein are hereby incorporated by reference to the same extent as if each was specifically and individually indicated to be incorporated by reference. In the case of inconsistencies, the present disclosure will prevail.

The following publications are hereby incorporated by reference in their entirety: U.S. Patent Publication Nos. 2004/0248221 and 2005/0032124.

### Definitions

The term “—(C₁-C₆)alkyl” as used herein, refers to a straight chain or branched non-cyclic hydrocarbon having from 1 to 6 carbon atoms. Representative straight chain —(C₁-C₆)alkyls include -methyl, -ethyl, -n-propyl, -n-butyl and -n-pentyl. Representative branched —(C₁-C₆)alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl and 3,3-dimethylbutyl.

The term “pharmaceutically acceptable salt” as used herein refers to a salt of an acid and a basic nitrogen atom of a compound of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes a hydrate of a compound of the present invention.

The premise of chemical genetics is such that small molecules can be used to identify pathways and proteins that underlie biological responses and events (Schreiber, et al, Bioorg. Med. Chem. 1998, 6: 1127-52; Stockwell B, et al, Nat Rev Genet 2000, 1:116-25; Stockwell, B, et al, Trends Biotechnol 2000, 18: 449-55). The ability of genotype-selective compounds to serve as molecular probes is based on said premise. In such example, the natural product rapamycin was previously observed to inhibit cell growth. This observation provided the foundation for the discovery of the mammalian Target of Rapamycin (mTOR) as a protein that regulates cell growth (Brown, et al., Nature 1994, 369: 756-758; Sabatini, et al., Cell 1994, 78: 35-43). Thus, two approaches, chemical and molecular genetics, have been combined in the present invention to discover pathways that are affected by mutations associated with human disorders and diseases (for example, HD and cancer), as well to identify compounds to treat such disorders and diseases.

A series of human tumor cells, in addition to cells harboring a mutation and phenotype related to a disease state, have been engineered with designated genetic elements. This allows for the identification of critical pathways whose disruption leads to a tumorigenic or disease-state phenotype (Hahn, et al., Nat Med 1999, 5: 1164-70; Lessnick, et al., Cancer Cell 2002, 1: 393-401; Hahn, et al., Nat Rev Cancer 2002, 2: 331-41). The present invention details the use of these experimentally engineered cells, which purportedly allow for the identification of genotype-selective agents (for example, from both known and novel compound sources) that exhibit synthetic lethality in the presence of specific cancer-related and disease-associated alleles. Thus, compounds exhibiting genotype-selective lethality may serve as molecular probes for signaling networks present in disease-state and tumorigenic cells. In addition, these compounds may subsequently be developed into therapeutically effective drugs. This invention also utilizes high-throughput screens developed to identify suppressors (for example, small molecules) of the toxicity of huntingtin (Htt) mutants in neuronal cells. The present invention discloses compounds identified by HTS methods described herein that promote viability of neuronal cells expressing mutant huntingtin. The genotype-selective compounds identified by HTS may serve as molecular probes of signaling networks present in neuronal cells from subjects having HD. In addition, these compounds may subsequently be developed into therapeutically effective agents to treat such subjects.

### Engineered Cell Lines

In one embodiment, the present invention relates to engineered tumorigenic cell lines. In another embodiment, the tumorigenic cells are human colon carcinoma cells expressing an E6 oncoprotein (for example, RKO-E6 cancer cells; ATCC # CRL-2577). The RKO-E6 cell line was generated by transfecting cells with a pCMV.3 plasmid containing an HPV E6 cDNA cassette. In the RKO-E6 cell line, HPV E6 inactivates p53, thus cells can overcome cell cycle checkpoint controls and become resistant to apoptotic events. In one embodiment of the invention, the colon cancer cell line (for example, RKO-E6 cells) serves as a model for E6-induced resistance to apoptosis. Results of a large-scale screen for compounds that enables this engineered tumorigenic cell line to overcome E6-induced resistance to apoptosis are described in Example 1.

In another embodiment, the present invention relates to engineered neuronal cell lines. In the present invention, the engineered neuronal cell lines serve as models for neurodegenerative disorders/disease, such as Huntington's Disease. In one embodiment, neuronal cells were engineered to express a mutant huntingtin protein. Non-limiting examples of neuronal cells that were engineered to express a mutant huntingtin protein include ST14A cells and PC12 cells as described in the present invention. In another embodiment, neuronal cells (for example, PC12 cells and ST14A cells) can be transfected with a cDNA of exon-1 of the human huntingtin gene, which contains 103 N-terminal polyQ repeats (Q103 huntingtin mutant). Results of a large-scale screen for compounds that enables this engineered tumorigenic cell line to resist the effects of mutant htt toxicity are described in Example 2.

### Methods of Compound Screening

The present invention relates to large-scale compound screens wherein engineered tumorigenic cell lines exposed to screening compounds display selective toxicity or growth inhibition (for example, proliferation of tumorigenic cells ceases). Large-scale screens may include screens wherein hundreds or thousands or tens of thousands of compounds are screened (for example, in a high-throughput format) for selective toxicity to engineered tumorigenic cells. In one embodiment of the invention, engineered tumorigenic cell lines can be colon cancer cells (for example, RKO-E6 cells).

In one embodiment, selective toxicity is determined by comparing the cell viability of control cells (for example, wild type, parental cells) and test cells (such as engineered tumorigenic cells) after contact with a candidate compound. A control cell is the same type of cell as test cells, except that the control cell has not been genetically manipulated to be tumorigenic. In another embodiment, control cells can be the parental primary cells from which the test cells are derived. In yet another embodiment, under the same experimental conditions as the test cells, control cells are contacted with the candidate compound.

In another embodiment of the present invention, the candidate compound can be selected from a compound library (for example, a combinatorial library). Cell viability (for example, that of engineered tumorigenic cells) may be determined by methods understood by one skilled in the art, such as Alamar Blue staining or detection with the dye calcein acetoxymethyl ester (calcein AM). In some embodiments, a dye (for example, calcein AM) is added to control and test cells after treatment with a candidate compound. In living cells, calcein AM is cleaved by intracellular esterases, and forms the anionic fluorescent derivative calcein. Calcein cannot permeate the cell membrane of live cells. Thus, live cells display a green fluorescence when incubated with calcein AM, whereas dead cells do not. The green fluorescence exhibited by living cells can be detected using methods known in the art and can thereby provide a measurement of cell viability.

In one embodiment, a compound identified as selectively promoting cell death in an engineered tumorigenic cell, such as a colon cancer cell, can be further characterized in an animal model. Non-limiting examples of animal models include, C. elegans, rats, mice, rabbits, and monkeys. Animal models, for example mice, can be non-transgenic (for example, wildtype) or transgenic animals. The effect of the candidate compound that induces selective toxicity in engineered tumorigenic cells can be further assessed in an animal model (for example, a mouse). Some effects that can be monitored include, but are not limited to, the ability of the compound to selectively induce cell death in tumorigenic cells in the animal model, as well as general toxicity effects to the animal.

In other embodiments of the invention, the invention relates to a method of identifying compounds that selectively suppresses cellular toxicity in engineered cells. In one embodiment, the engineered cells are neuronal cells. In another embodiment, the engineered neuronal cells express a mutant huntingtin (htt) protein.

In some embodiments, the invention relates to a method of identifying a compound that suppresses the cellular toxicity of a mutant huntingtin protein in engineered cells (for example, neuronal cells). The method comprises contacting test cells (for example, engineered neuronal cells expressing a mutant huntingtin protein) with a candidate compound; then, determining the viability of test cells contacted with the candidate compound; and comparing the viability of test cells with the viability of an appropriate control (such as non-transfected, parental neuronal cells). An appropriate control is the same type of cell as that of test cells except that the control cell is not manipulated to express a protein that is toxic to the cell. The control cells may be the parental primary cells from which the test cells are derived.

In another embodiment, a compound that selectively suppresses cellular toxicity (e.g., huntingtin-induced cellular toxicity) in engineered neuronal cells can be identified when the viability of the test cells is more than that of the control cells. Control cells are contacted with the candidate compound under the same experimental conditions as the test cells.

In some embodiments, the genotype-selective compounds of the invention (for example, anti-tumor agents or anti-HD agents) can be any chemical (element, molecule, compound, drug), made synthetically, made by recombinant techniques established in the art, or isolated from a natural source by methods known in the art. For example, these compounds can be sugars, hormones, peptides, polypeptides, or nucleic acid molecules (such as anti-sense or RNAi nucleic acid molecules). In addition, these compounds can be small molecules or molecules of greater complexity (for example, those made by combinatorial chemistry), compiled into libraries. Such combinatorial libraries can comprise, for example, amines, amides, alcohols, esters, alkyl halides, aldehydes, ethers, and other classes of organic compounds. These compounds can also be natural or genetically engineered products isolated from lysates or growth media of cells (for example, bacterial, animal, yeast, or plant cells). Exposing such compounds to a test system can be in either an isolated form or as mixtures of compounds (2 or 3 or more compounds), especially in the initial screening steps.

### Compounds of the Invention

In one embodiment, the present invention describes methods by which to identify compounds with increased potency and activity in the presence of specific genetic elements. Previous reports address the possibility to identify such genotype-selective compounds with respect to one genetic element of interest (Simons, et al, Genome Res 2001, 11: 266-73; Stockwell B, et al, Chem Biol 1999, 6: 71-83; Torrance, et al, Nat Biotechnol 2001, 19: 940-5). In one embodiment, the invention provides for a systematic testing of increased cell death using more than 27,000 compounds in combination with apoptosis-inducing drugs (such as anthracyclines), and one or more cancer-related genetic elements. In another embodiment, the invention provides for a systematic testing of resistance to cellular toxicity caused by mutant proteins using more than 40,000 compounds and a neurodegenerative disorder-related genetic element, such an htt mutant protein.

In one embodiment, secondary acylated amines, referred to herein as indoxins, in combination with anthracyclines can be used to induce cell death in a tumor cell wherein contact of the tumor cell with an anthracycline and an indoxin results in cell death. In some embodiments, the tumor cells of the invention are engineered human cancer colon cells (such as RKO-E6 cells). In another embodiment of the invention, an anthracycline can be doxorubicin. Tumorigenic cells in which lethality may be induced by indoxin activity can include tumorigenic cells with an activated E6 oncoprotein-mediated pathway.

In certain embodiments, inhibitors (suppressors) of neuronal cell death induced by mutant huntingtin (htt) proteins (referred to herein as revertins) were identified using the screening methods of the present invention. About 50 compounds were identified that selectively prevented mutant huntingtin-induced death of neuronal cells when a combinatorial library of about 47,000 compounds was screened. In addition, a small number of compounds were identified to increase the viability of mutant huntingtin-expressing neuronal cells. These “revertins,” which are suppressors of mutant huntingtin-induced neuronal cell death, include, but are not limited to, secondary acylated amines, such as Revertin-20 (TABLE 3).

Accordingly, exemplary compounds and pharmaceutically acceptable salts of compounds useful in the methods of the present invention are outlined below.

### Compounds of Formula (I):

In one embodiment, the present invention provides compounds of the Formula (I):

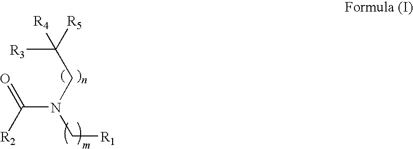

and pharmaceutically acceptable salts thereof, wherein

R₁ is

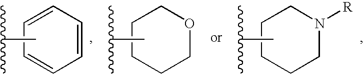

each optionally substituted with one or more C₁-C₆ alkyl, —O(C₁-C₆ alkyl), —NH(C₁-C₆ alkyl), —N(C₁-C₆ alkyl)₂, —F or —CF₃ groups;

R₂ is C₁-C₆ alkyl, —CF₃,

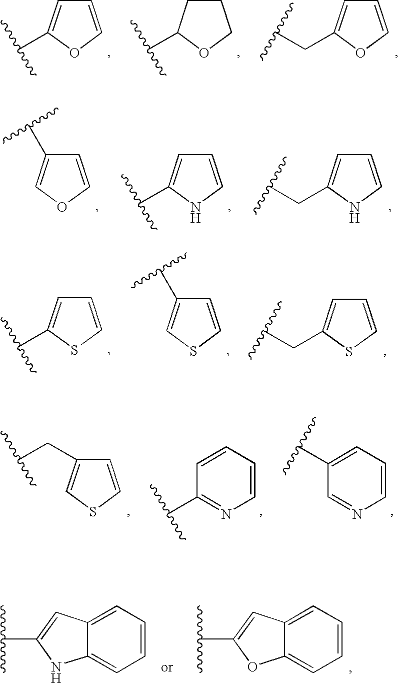

each ring optionally substituted with one or more C₁-C₆ alkyl, —O(C₁-C₆ alkyl) or —NO₂ groups;

R₃ is

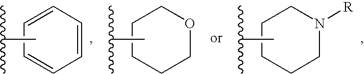

each optionally substituted with one or more C₁-C₆ alkyl, —O(C₁-C₆ alkyl), —NH(C₁-C₆ alkyl), —N(C₁-C₆ alkyl)₂, —F or —CF₃ groups;
one of R₄ and R₅ is hydrogen and the other of R₄ and R₅ is

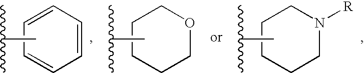

each optionally substituted with one or more C₁-C₆ alkyl, —O(C₁-C₆ alkyl), —NH(C₁-C₆ alkyl), —N(C₁-C₆ alkyl)₂, —F or —CF₃ groups;

or R₄ and R₅ taken together with the carbon to which they are attached form

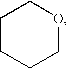

which is optionally substituted with one or more C₁-C₆ alkyl, —O(C₁-C₆ alkyl), —NH(C₁-C₆ alkyl), —N(C₁-C₆ alkyl)₂, —F or —CF₃ groups;

n is 1-3; and

m is 0-2.

In one embodiment, R₁ is

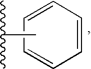

optionally substituted with one or more C₁-C₆ alkyl, —O(C₁-C₆ alkyl), —NH(C₁-C₆ alkyl), —N(C₁-C₆ alkyl)₂, —F or —CF₃ groups.

In one embodiment, R₂ is

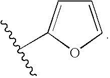

In one embodiment, R₃ is

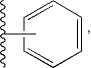

optionally substituted with one or more C₁-C₆ alkyl, —O(C₁-C₆ alkyl), —NH(C₁-C₆ alkyl), —N(C₁-C₆ alkyl)₂, —F or —CF₃ groups.

In one embodiment, R₃ is

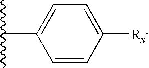

wherein Rₓ is hydrogen, C₁-C₆ alkyl or —O(C₁-C₆ alkyl).

In one embodiment, R₃ is

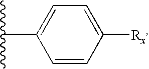

wherein Rₓ is hydrogen, —CH₃ or —OCH₃.

In one embodiment, R₄ and R₅ taken together with the carbon to which they are attached form

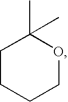

optionally further substituted with one or more C₁-C₆ alkyl, —O(C₁-C₆ alkyl), —NH(C₁-C₆ alkyl), —N(C₁-C₆ alkyl)₂, —F or —CF₃ groups.

In one embodiment, R₄ and R₅ taken together with the carbon to which they are attached form

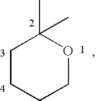

wherein the carbon to which R₄ and R₅ are attached is in the 4-position relative to the oxygen atom of the tetrahydropyran ring, and the tetrahydropyran ring is optionally further substituted with one or more C₁-C₆ alkyl, —O(C₁-C₆ alkyl), —NH(C₁-C₆ alkyl), —N(C₁-C₆ alkyl)₂, —F or —CF₃ groups.

In one embodiment, one of R₄ and R₅ is

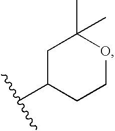

optionally further substituted with one or more C₁-C₆ alkyl, —O(C₁-C₆ alkyl), —NH(C₁-C₆ alkyl), —N(C₁-C₆ alkyl)₂, —F or —CF₃ groups.

In one embodiment, one of R₄ and R₅ is

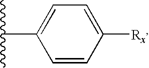

wherein Rₓ is hydrogen, C₁-C₆ alkyl or —O(C₁-C₆ alkyl).

In one embodiment, one of R₄ and R₅ is

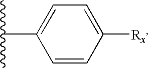

wherein Rₓ is hydrogen, —CH₃ or —OCH₃.

In one embodiment, the compound of Formula (I) is

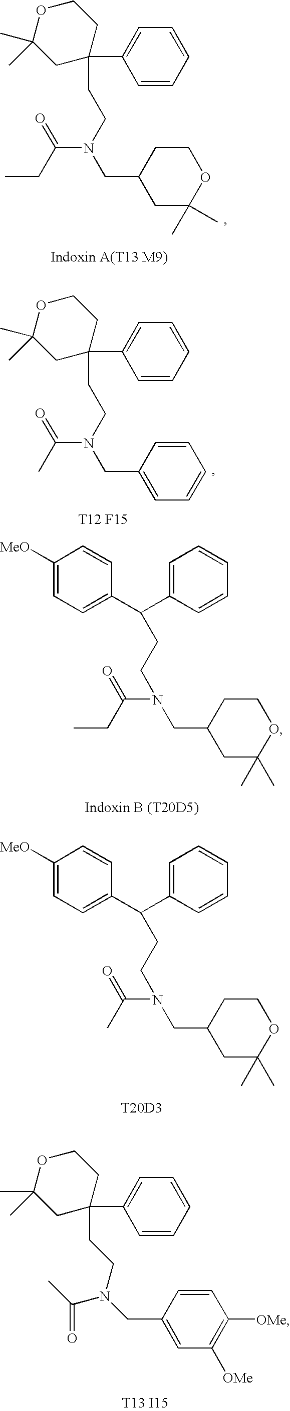

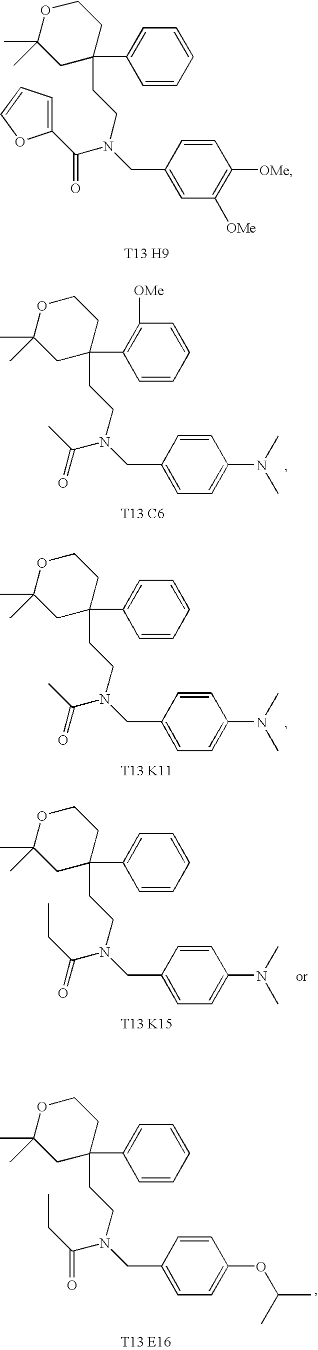

or a pharmaceutically acceptable salt thereof.

In one embodiment, the compound of Formula (I) is

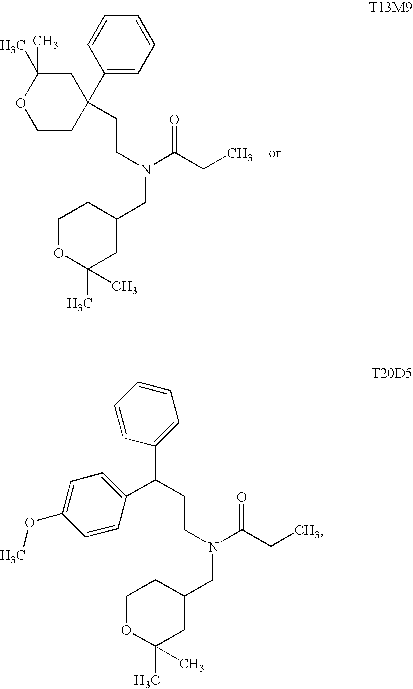

or a pharmaceutically acceptable salt thereof.

In one embodiment, the compound of Formula (I) is

| Compound | |
|---|---|
| Name | Structure |
| 164-1 | 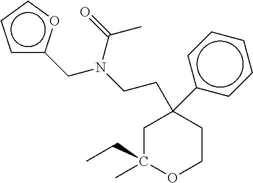 |
| 164-3 | 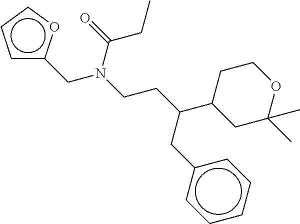 |
| 164-4 | 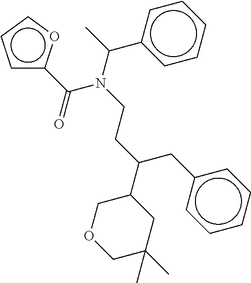 |
| 164-5 | 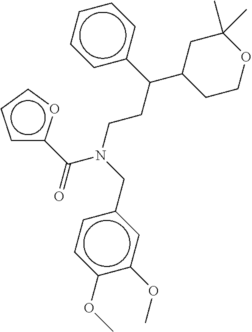 |
| 164-6 | 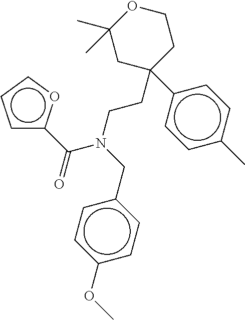 |
| 164-8 | 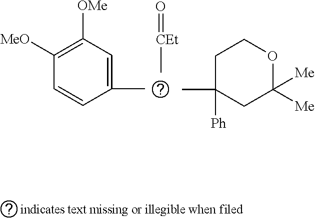 |
| 164-9 | 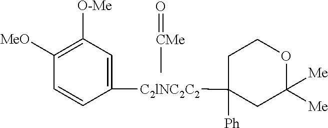 |
| 164-10 | 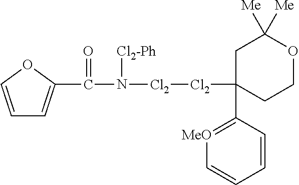 |
| 164-13 | 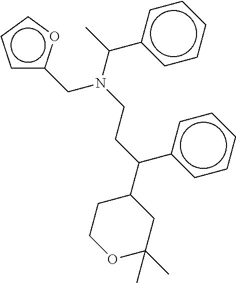 |
| 164-14 | 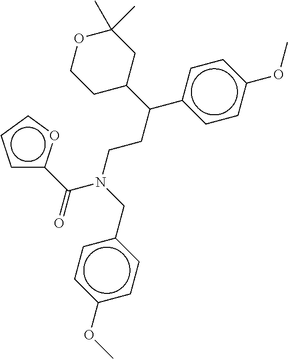 |
| 164-15 | 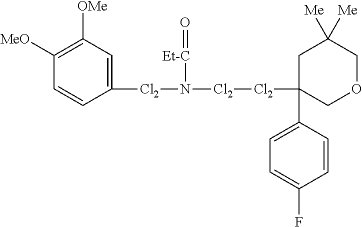 |
| 164-16 | 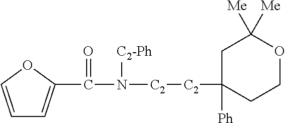 |
| 164 | 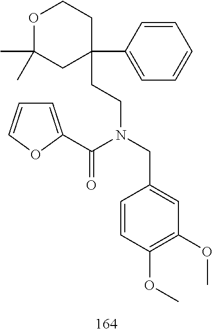 |
| 164-A | 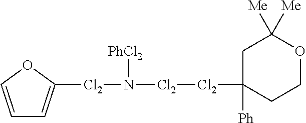 |
| 164-B | 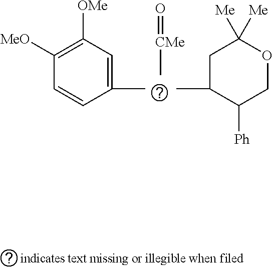 |
| 164-C | 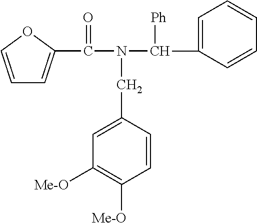 |
| 164-D | 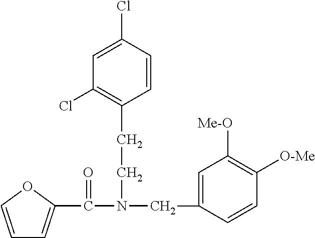 |
| 164-E | 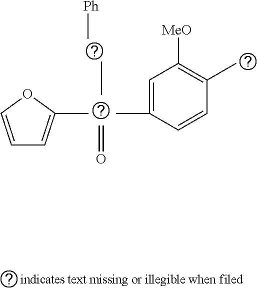 |
| 372 | 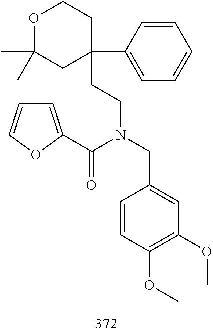 |
| 373 | 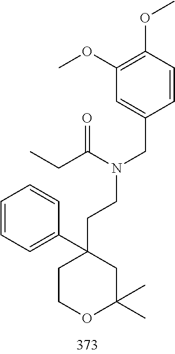 |
| 375 | 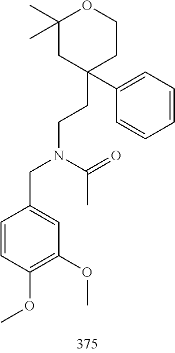 |
| 377 | 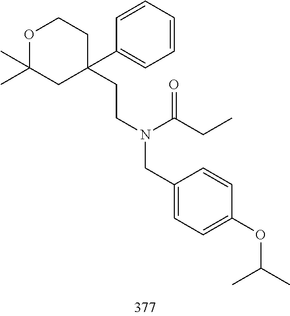 |
| 378 | 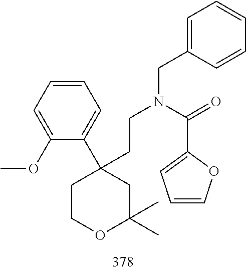 |
| 383 | 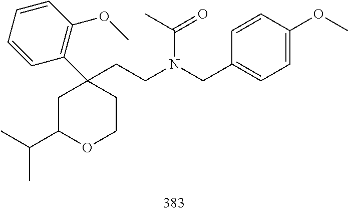 |
| 384 | 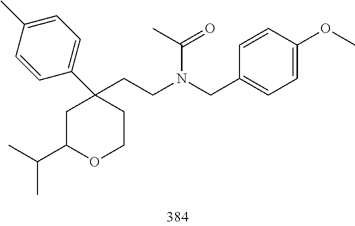 |
| 386 | 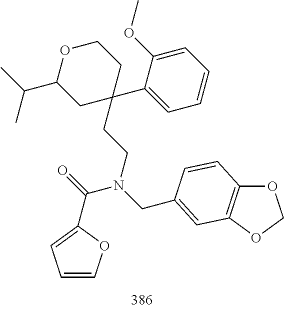 |
| 387 | 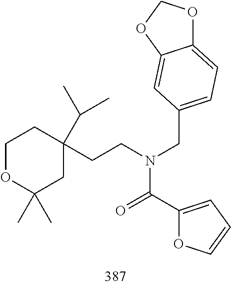 |
| 164-F | 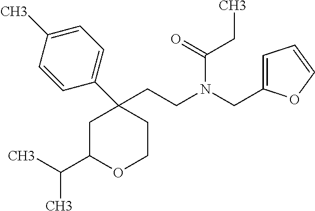 |

or a pharmaceutically acceptable salt thereof.

In one embodiment, the compound is Compound 164-5, Compound 164-16 or Compound 164, or a pharmaceutically acceptable salt thereof.

In one embodiment, the compound or pharmaceutically acceptable salt of the compound of Formula (I) is synthesized using combinatorial methodology.

In one embodiment, the compound or pharmaceutically acceptable salt of the compound of Formula (I) is derivatized with a label.

In one embodiment, the label comprises biotin.

In one embodiment, the label comprises a fluorescent moiety.

In one embodiment, the label comprises fluorescein.

In one embodiment, the label comprises benzophenone.

### Compounds of Formula (II):

In one embodiment, the present invention provides compounds of the Formula (II):

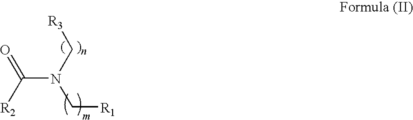

and pharmaceutically acceptable salts thereof, wherein

R₁ is

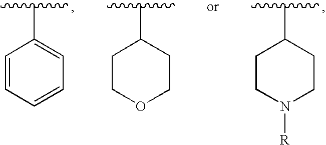

each optionally substituted with one or more C₁-C₆ alkyl, —O(C₁-C₆ alkyl), —NH(C₁-C₆ alkyl), —N(C₁-C₆ alkyl)₂, —F or —CF₃ groups;

R is hydrogen, C₁-C₆ alkyl or —CF₃;

R₂ is

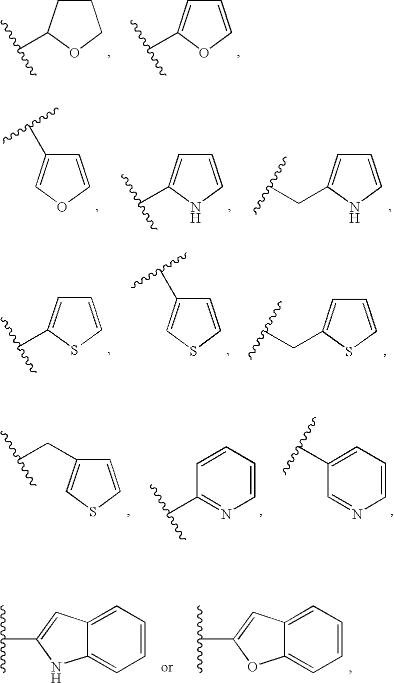

each ring optionally substituted with one or more C₁-C₆ alkyl, —O(C₁-C₆ alkyl) or —NO₂ groups;

or R₂ is

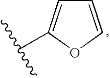

having one or more C₁-C₆ alkyl, —O(C₁-C₆ alkyl) or —NO₂ groups;

R₃ is

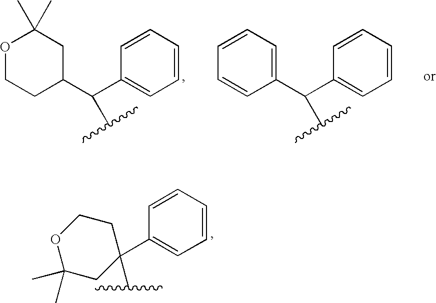

each ring optionally further substituted with one or more C₁-C₆ alkyl or —O(C₁-C₆ alkyl) groups;

n is 1-3; and

m is 0-2.

In one embodiment, n is 1 or 2.

In another embodiment, m is 1.

In one embodiment, R₁ is

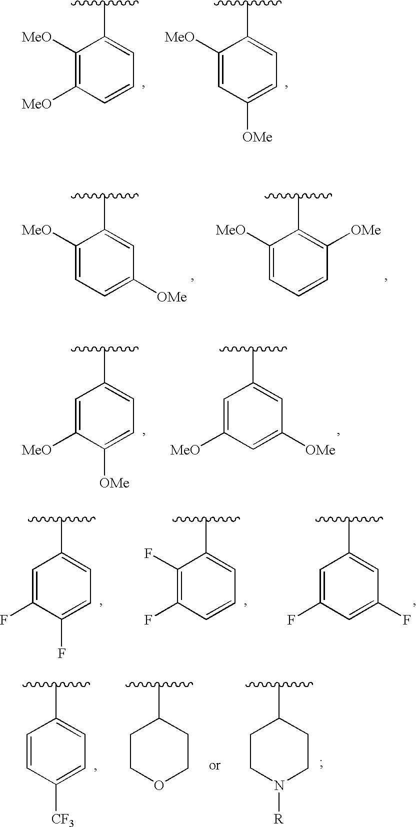

and R is hydrogen, C₁-C₆ alkyl or —CF₃.

In one embodiment, R₂ is

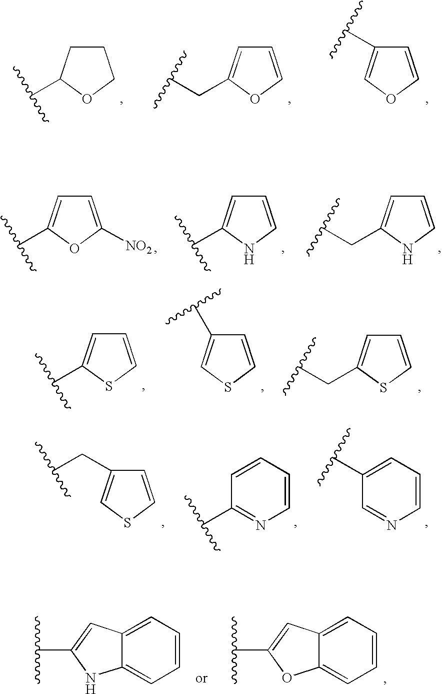

In one embodiment, or R₂ is

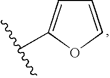

having one or more C₁-C₆ alkyl, —O(C₁-C₆ alkyl) or —NO₂ groups.

In one embodiment, R₃ is

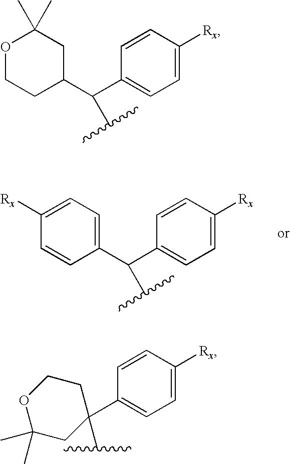

wherein Rₓ is hydrogen, C₁-C₆ alkyl or —O(C₁-C₆ alkyl).

In one embodiment, R₃ is

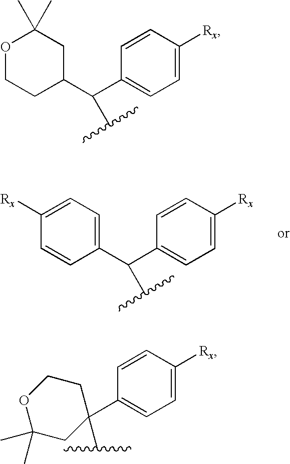

wherein Rₓ is hydrogen, —CH₃ or —OCH₃.

In one embodiment, R₃ is

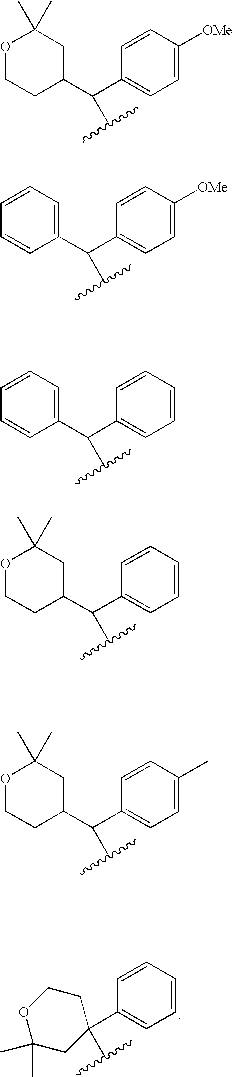

In one embodiment, the compound or pharmaceutically acceptable salt of the compound of Formula (II) is synthesized using combinatorial methodology.

In one embodiment, the compound or pharmaceutically acceptable salt of the compound of Formula (II) is derivatized with a label.

In one embodiment, the label comprises biotin.

In one embodiment, the label comprises a fluorescent moiety.

In one embodiment, the label comprises fluorescein.

In one embodiment, the label comprises benzophenone.

In one embodiment, the present invention provides compounds of Formula (III):

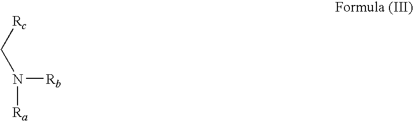

and pharmaceutically acceptable salts thereof,
wherein
Rₐ is

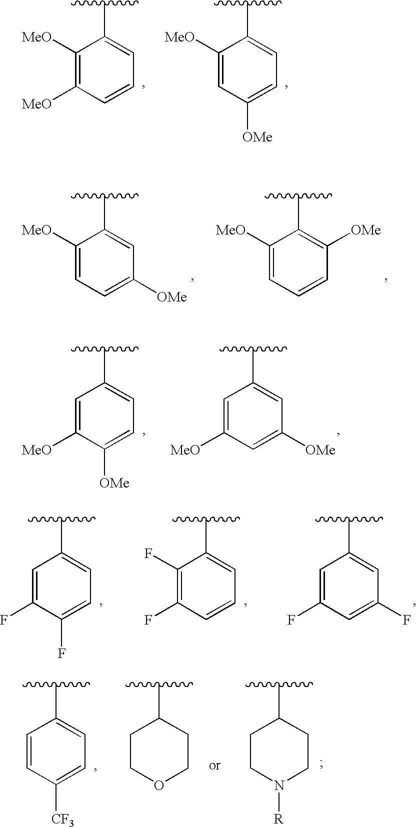

R is C₁-C₆ alkyl or haloalkyl;
R_{b} is

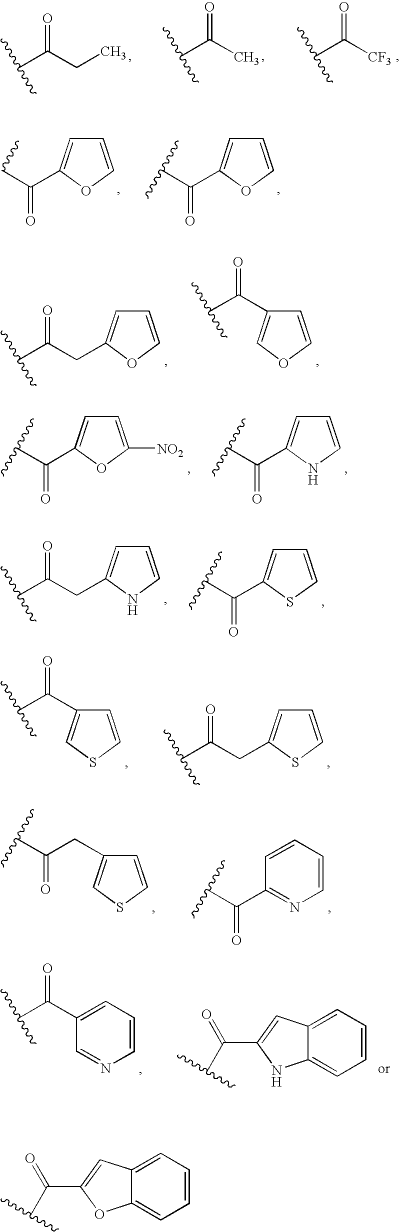

and wherein the

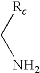

component is:

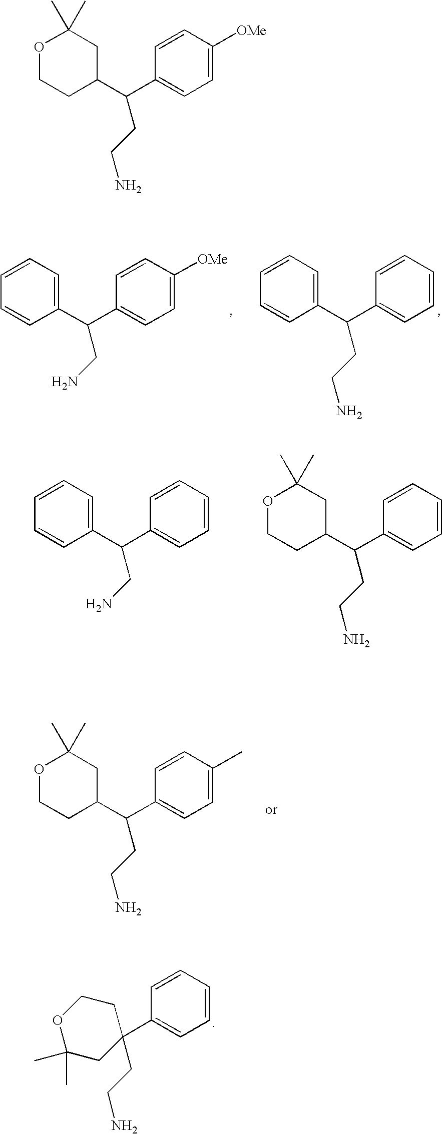

In one embodiment, the compound or pharmaceutically acceptable salt of the compound of Formula (III) is synthesized using combinatorial methodology.

In one embodiment, the compound or pharmaceutically acceptable salt of the compound of Formula (III) is derivatized with a label.

In one embodiment, the label comprises biotin.

In one embodiment, the label comprises a fluorescent moiety.

In one embodiment, the label comprises fluorescein.

In one embodiment, the label comprises benzophenone.

### Compounds of Formula (X)

In one embodiment, the invention provides compounds of Formula (X):

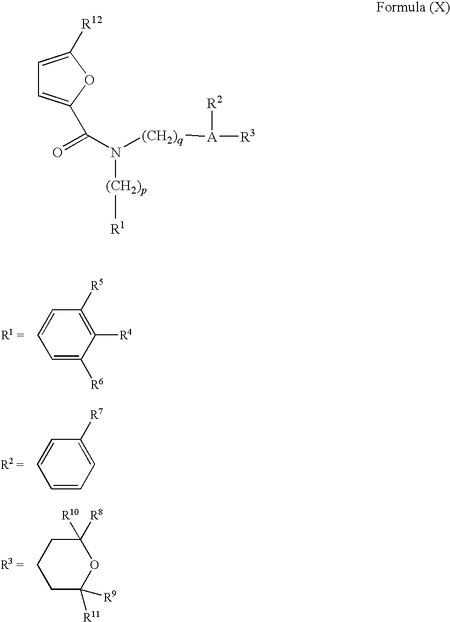

Or a pharmaceutically acceptable salt thereof.
In formula X, A may be absent or (CH);
p=0 or 1-3, and is particularly 1;
q=1-4 and is particularly 2;
R⁴ may be H or (C₁₋₄) alkoxy, such as but not limited to methoxy and ethoxy, and in particular is methoxy;
R⁵ may be H or (C₁₋₄) alkoxy, such as but not limited to methoxy and ethoxy, and in particular is methoxy;
R⁶ may be H or (C₁₋₄) alkoxy, such as but not limited to methoxy and ethoxy, and in particular is methoxy;
however, of R⁴, R⁵, and R⁶, at least one is H;
R⁷ may be H or (C₁₋₄) alkyl or (C₁₋₄) alkoxy, and is particularly methyl;
R⁸ may be H or (C₁₋₄) alkyl, and is particularly isopropyl or isobutyl;
R⁹ may be H or (C₁₋₄) alkyl, and is particularly isopropyl or isobutyl;
if R⁸ is (C₁₋₄) alkyl, R⁹ is particularly H;
if R⁹ is (C₁₋₄) alkyl, R⁸ is particularly H;
if R⁸ or R⁹ is methyl or ethyl, the other substituent of the ring carbon to which it is linked, R¹⁰ or R¹¹, respectively may be H, methyl, or ethyl; otherwise R¹⁰ or R¹¹ is H; and R¹² may be H, F, Cl, I, or Br.

Examples of compounds having Formula (X) are shown in Table 4. Compounds of Formula (X) include, for example, Compound 164, Compound 164-2, Compound 164-3, Compound 164-5, and Compound 164-6, as shown in Table 4. In a specific non-limiting embodiment, the local concentration of a compound of Formula (X) or a pharmaceutically acceptable salt thereof, at a neuron to be treated is between about 0.2 micromolar and about 50 mM, or between about 0.1 mM and about 50 mM.

Table 4 illustrates exemplary compounds of Formula (X) according to the present invention along with their respective cell growth inhibition activities.

TABLE 4
Structures and Activities of Exemplary Compounds

| ID # | | | | | | | | | |
|---|---|---|---|---|---|---|---|---|---|
| 164 | 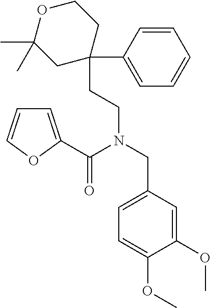 | | | | | | | | |
| 164-1 | 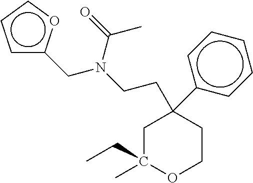 | | | | | | | | |
| 164-2 | 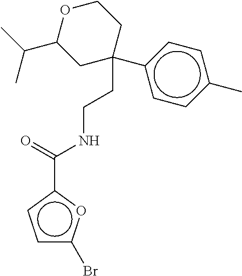 | | | | | | | | |
| 164-3 | 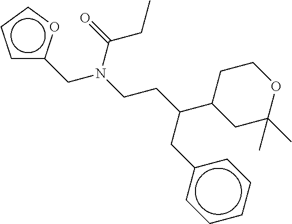 | | | | | | | | |
| 164-4 | 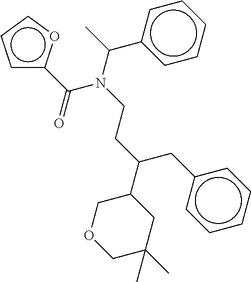 | | | | | | | | |
| 164-5 | 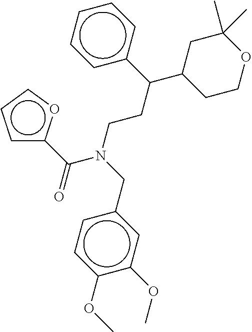 | | | | | | | | |
| 164-6 | 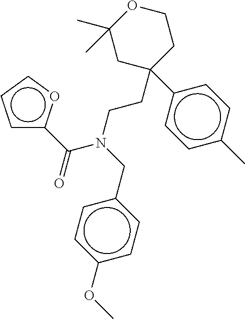 | | | | | | | | |
| 164-7 | 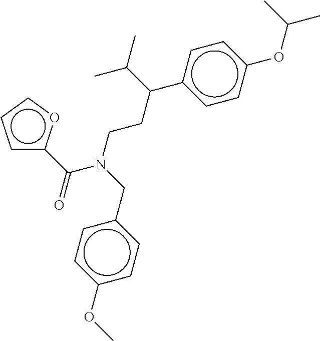 | | | | | | | | |
| 164-8 | 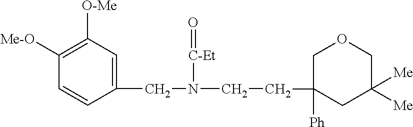 | | | | | | | | |
| 164-9 | 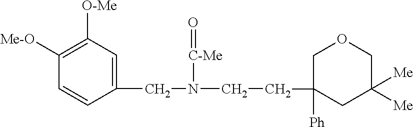 | | | | | | | | |
| 164-10 | 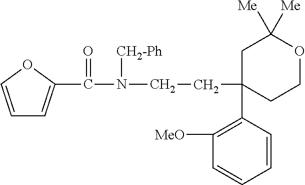 | | | | | | | | |
| 164-11 | 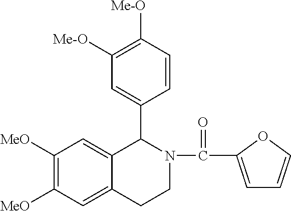 | | | | | | | | |
| 164-12 | 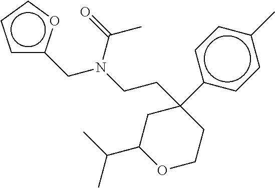 | | | | | | | | |
| 164-13 | 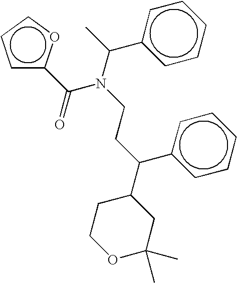 | | | | | | | | |
| 164-14 | 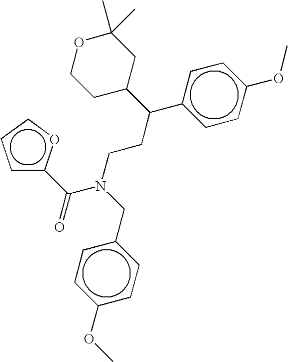 | | | | | | | | |
| 164-15 | 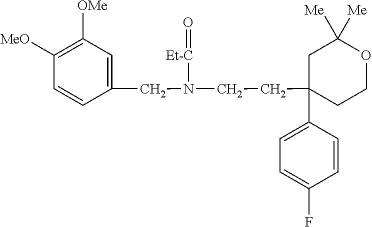 | | | | | | | | |
| 164-16 | 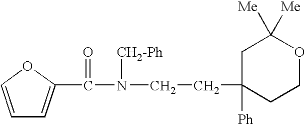 | | | | | | | | |
| 164-17 | 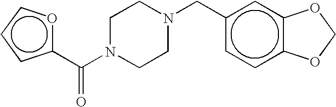 | | | | | | | | |
| 164-18 | 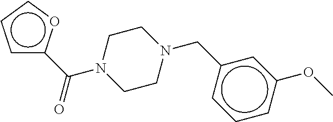 | | | | | | | | |
| 164-19 | 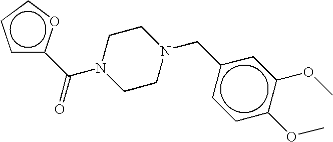 | | | | | | | | |
| | Conc. (in | | CA | | | | | | |
| | mother, | | Index | Registry/ | Supplier/ | Max % | Max % | | |
| ID | mM) | MW | Name | CNC# | Catalog # | Rescue | Toxicity | EC₅₀ | TC₅₀ |
| 164 | 78.52 | 477.6 | | | | 103.97 | 1.27 | 10 mM | N/A |
| 164-1 | 135.32 | 369.5 | | CNC- | IBS | 67.24 | 59.48 | 2 mM | 8 mM |
| | | | | 3637184 | STOCK1 | | | | |
| | | | | | N-31690 | | | | |
| 164-2 | 115.11 | 434.37 | | CNC- | IBS | 122.29 | 53.69 | 0.44 mM | N/A |
| | | | | 61461716 | STOCK5S- | | | | |
| | | | | | 67216 | | | | |
| 164-3 | 125.77 | 397.56 | | CNC- | IBS | 83.88 | 56.80 | 0.2 mM | 8 mM |
| | | | | 3635859 | STOCK1 | | | | |
| | | | | | N-30365 | | | | |
| 164-4 | 108.78 | 459.63 | | CNC- | IBS | 59.18 | 64.22 | NA | 0.4 mM |
| | | | | 48649047 | STOCK5S- | | | | |
| | | | | | 31827 | | | | |
| 164-5 | 25.43 | 491.63 | N-(3,4- | CNC- | ChemBridge | 155.77 | 18.43 | 0.2 mM | 6 mM |
| | | | dimethoxybenzyl)- | 17894918 | 5936310 | | | | |
| | | | N-[3-(2,2- | | | | | | |
| | | | dimethyltetrahydro- | | | | | | |
| | | | 2H-pyran-4-yl)-3- | | | | | | |
| | | | phenylpropyl]-2- | | | | | | |
| | | | furamide | | | | | | |
| 164-6 | 54.16 | 461.6 | | CNC- | IBS | 126.64 | 60.14 | 0.1 mM | 3 mM |
| | | | | 376247 | STOCK1 | | | | |
| | | | | | N-13863 | | | | |
| 164-7 | 111.21 | 449.59 | | CNC- | IBS | 76.96 | 63.90 | 0.1 mM | 0.4 mM |
| | | | | 48657640 | STOCK5S- | | | | |
| | | | | | 37746 | | | | |
| 164-8 | 113.74 | 439.59 | Propanamide, N- | 350993- | IBS | 51.48 | 59.46 | NA | 14 mM |
| | | | [(3,4- | 68-9 | STOCK1 | | | | |
| | | | dimethoxyphenyl) | | N-13473 | | | | |
| | | | methyl]-N-[2- | | | | | | |
| | | | (tetrahydro-2,2- | | | | | | |
| | | | dimethyl-4-phenyl- | | | | | | |
| | | | 2H-pyran-4- | | | | | | |
| | | | yl)ethyl]-(9CI) | | | | | | |
| 164-9 | 39.16 | 425.56 | Acetamide, N- | 350993- | IBS | 28.56 | 19.60 | 10 mM | NA |
| | | | [(3,4- | 71-4 | STOCK1 | | | | |
| | | | dimethoxyphenyl) | | N-14117 | | | | |
| | | | methyl]-N-[2- | | | | | | |
| | | | (tetrahydro-2,2- | | | | | | |
| | | | dimethyl-4-phenyl- | | | | | | |
| | | | 2H-pyran-4- | | | | | | |
| | | | yl)ethyl[-(9CI) | | | | | | |
| 164-10 | 111.71 | 447.57 | 2- | 672899- | IBS | 83.35 | 62.27 | 0.2 mM | 1.7 mM |
| | | | Furancarboxamide, | 99-9 | STOCK1 | | | | |
| | | | N-(phenylmethyl)- | | N-20498 | | | | |
| | | | N-[2-[tetrahydro-4- | | | | | | |
| | | | (2-methoxyphenyl)- | | | | | | |
| | | | 2,2-dimethyl-2H- | | | | | | |
| | | | pyran-4-yl]ethyl]- | | | | | | |
| | | | (9CI) | | | | | | |
| 164-11 | 59.04 | 423.46 | Isoquinoline, 1- | 780821- | Chembridge | 7.95 | 50.50 | NA | 30 mM |
| | | | (3,4- | 36-5 | 7929284 | | | | |
| | | | dimethoxyphenyl)- | | | | | | |
| | | | 2-(2- | | | | | | |
| | | | furanylcarbonyl)- | | | | | | |
| | | | 1,2,3,4-tetrahydro- | | | | | | |
| | | | 6,7-dimethoxy- | | | | | | |
| | | | (9CI) | | | | | | |
| 164-12 | 65.18 | 383.53 | N-(2-furylmethyl)- | CNC- | Chembridge | 27.11 | 60.37 | NA | 8 mM |
| | | | N-{2-[2-isopropyl- | 26583579 | 7921510 | | | | |
| | | | 4-(4- | | | | | | |
| | | | methylphenyl) | | | | | | |
| | | | tetrahydro-2H- | | | | | | |
| | | | pyran-4- | | | | | | |
| | | | yl]ethyl}acetamide | | | | | | |
| 164-13 | 28.05 | 445.6 | N-[3-(2,2- | CNC- | Chembridge | 26.53 | 55.74 | NA | 4 mM |
| | | | dimethyltetrahydro- | 25462812 | 7834631 | | | | |
| | | | 2H-pyran-4-yl)-3- | | | | | | |
| | | | phenylpropyl]-N- | | | | | | |
| | | | (1-phenylethyl)-2- | | | | | | |
| | | | furamide | | | | | | |
| 164-14 | 50.85 | 491.63 | N-[3-(2,2- | CNC- | Chembridge | 86.87 | 53.80 | 0.2 mM | 2 mM |
| | | | dimethyltetrahydro- | 17908402 | 6545545 | | | | |
| | | | 2H-pyran-4-yl)-3- | | | | | | |
| | | | (4- | | | | | | |
| | | | methoxyphenyl) | | | | | | |
| | | | propyl]-N-(4- | | | | | | |
| | | | methoxybenzyl)-2- | | | | | | |
| | | | furamide | | | | | | |
| 164-15 | 27.32 | 457.58 | Propanamide, N- | 305867- | Chembridge | 48.92 | 45.74 | 4 mM | 27 mM |
| | | | [(3,4- | 67-8 | 6156308 | | | | |
| | | | dimethoxyphenyl) | | | | | | |
| | | | methyl]-N-[2-[4-(4- | | | | | | |
| | | | fluorophenyl) | | | | | | |
| | | | tetraydro- | | | | | | |
| | | | 2,2-dimethyl- | | | | | | |
| | | | 2H-pyran-4- | | | | | | |
| | | | yl}ethyl]-(9CI) | | | | | | |
| 164-16 | 29.94 | 417.54 | 2- | 303728- | Chembridge | 54.31 | 53.14 | 0.2 μM | 8 μM |
| | | | Furancarboxamide, | 92-9 | 6140216 | | | | |
| | | | N-(phenylmethyl)- | | | | | | |
| | | | N-[2-(tetrahydro- | | | | | | |
| | | | 2,2-dimethyl-4- | | | | | | |
| | | | phenyl-2H-pyran-4- | | | | | | |
| | | | yl)ethyl]-(9CI) | | | | | | |
| 164-17 | 39.77 | 314.34 | 1-(1,3-benzodioxol- | CNC- | Chembridge | 24.20 | 31.31 | N/A | N/A |
| | | | 5-ylmethyl)-4-(2- | 5884043 | 5818290 | | | | |
| | | | furoyl)piperazine | | | | | | |
| 164-18 | 41.62 | 300.36 | 1-(2-furoyl)-4-(3- | CNC- | Chembridge | 56.97 | 12.00 | N/A | N/A |
| | | | methoxybenzyl) | 5885197 | 5834202 | | | | |
| | | | piperazine | | | | | | |
| 164-19 | 37.84 | 330.38 | 1-(3,4- | CNC- | Chembridge | 45.96 | 27.77 | 18.9 mM | N/A |
| | | | dimethoxybenzyl)- | 5885053 | 5832322 | | | | |
| | | | 4-(2- | | | | | | |
| | | | furoyl)piperazine | | | | | | |

In one embodiment, the compound or pharmaceutically acceptable salt of the compound of Formula (X) is synthesized using combinatorial methodology.

In one embodiment, the compound or pharmaceutically acceptable salt of the compound of Formula (X) is derivatized with a label.

In one embodiment, the label comprises biotin.

In one embodiment, the label comprises a fluorescent moiety.

In one embodiment, the label comprises fluorescein.

In one embodiment, the label comprises benzophenone.

Compounds and pharmaceutically acceptable salts of compounds of Formula (X) are also useful in the methods of the present invention.

### Other Compounds of the Invention

In one embodiment, the present invention provides compounds as follows:

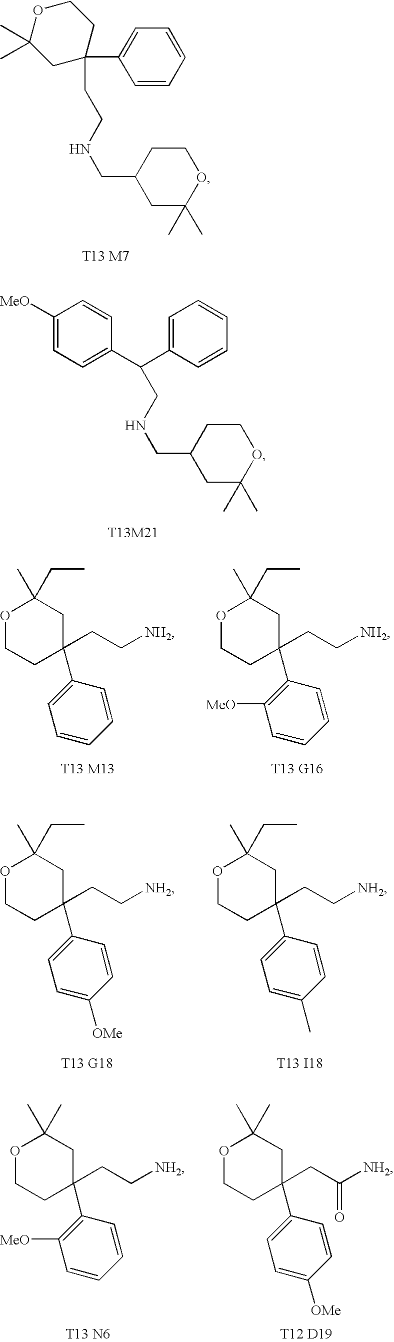

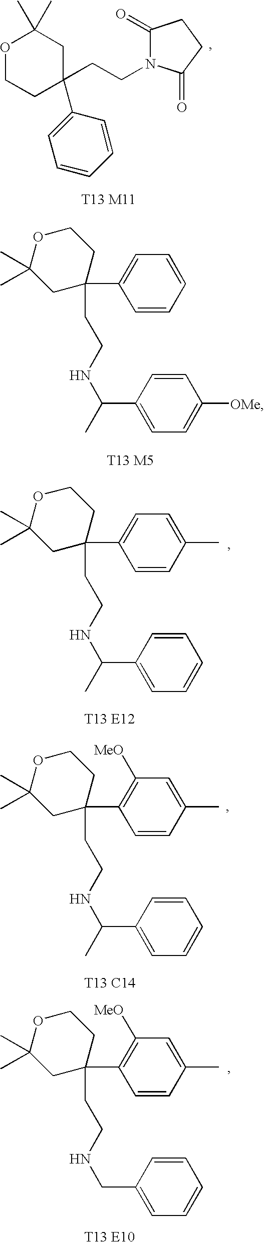

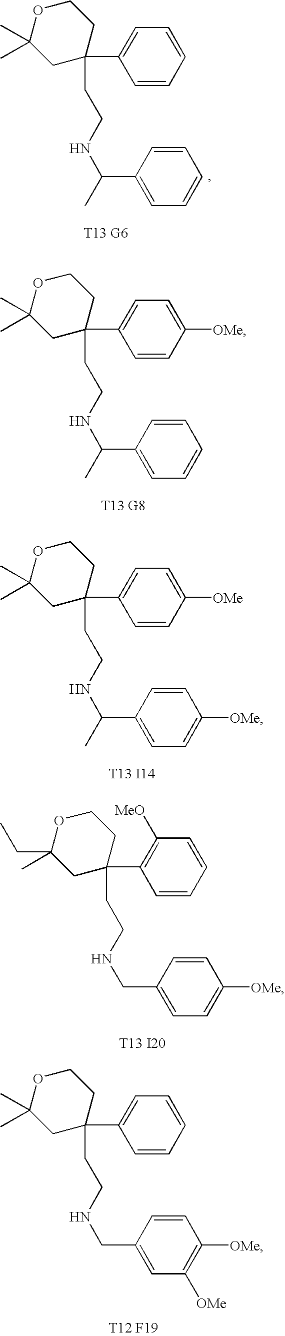

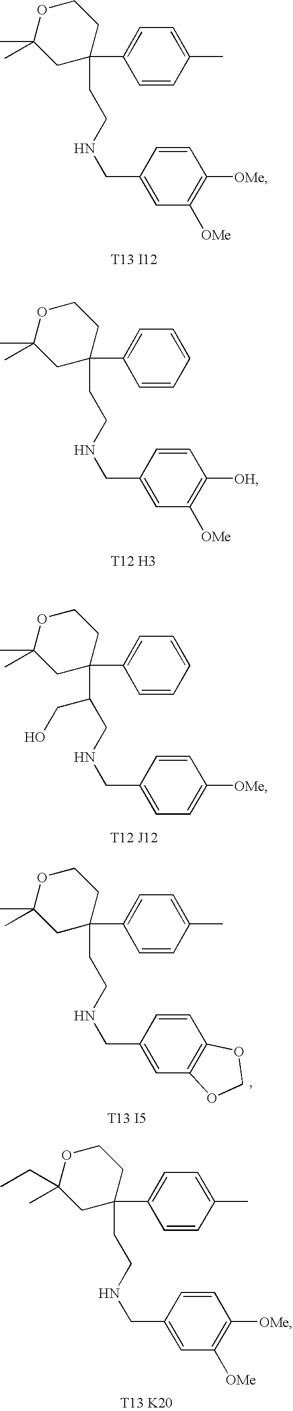

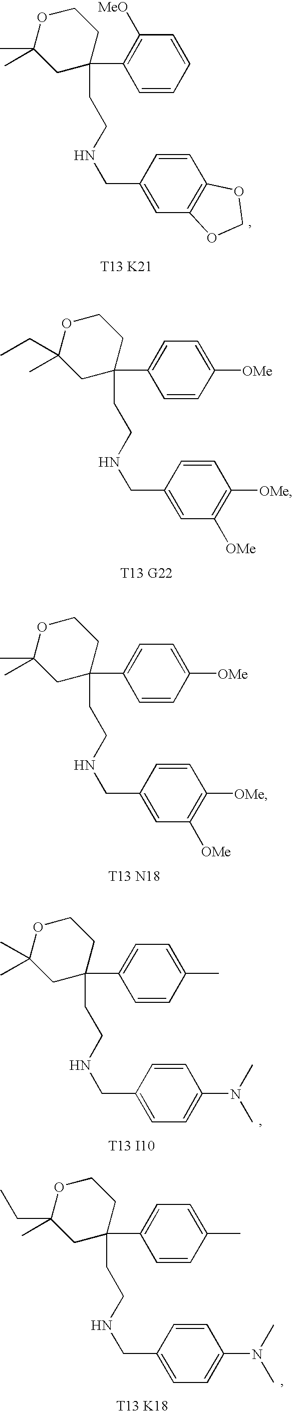

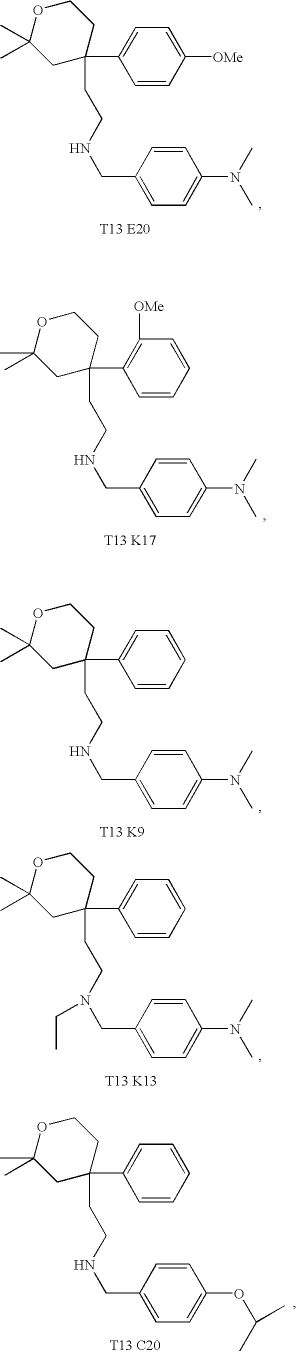

and pharmaceutically acceptable salts thereof.

In another embodiment, the present invention provides compounds as follows:

| Compound | |
|---|---|
| Name | Structure |
| 164-2 | 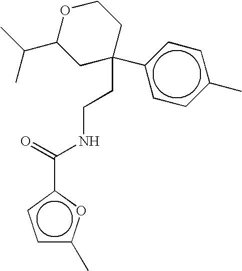 |
| 164-7 | 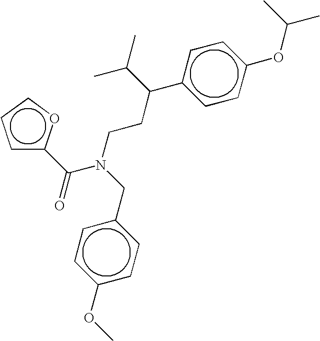 |
| 164-11 | 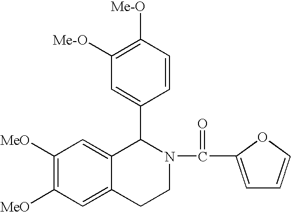 |
| 164-12 | 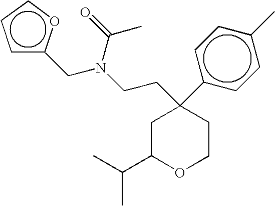 |
| 164-17 | 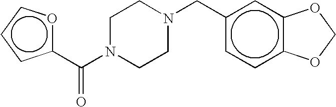 |
| 164-18 | 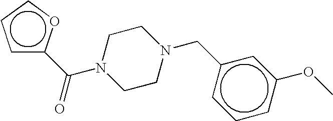 |
| 164-19 | 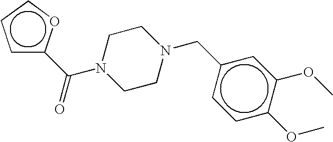 |
| 164-G | 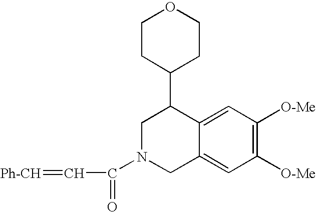 |
| 164-H | 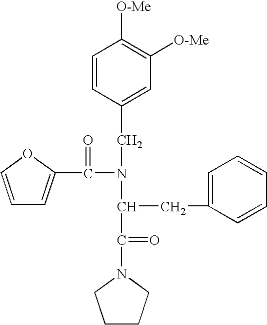 |
| 164-I | 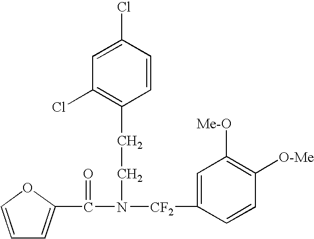 |
| 164-J | 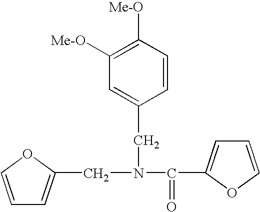 |
| 164-K | 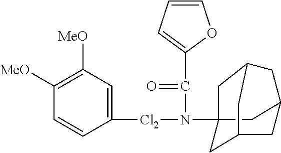 |
| 164-L | 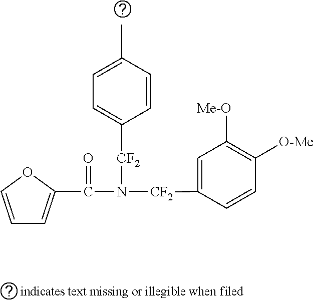 |
| 164-M | 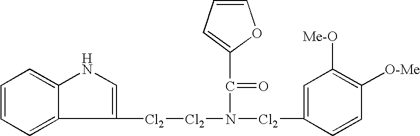 |
| 371 | 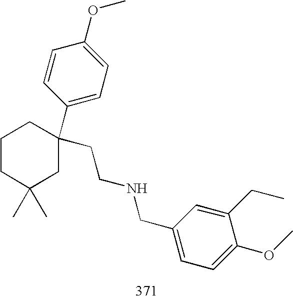 |
| 376 | 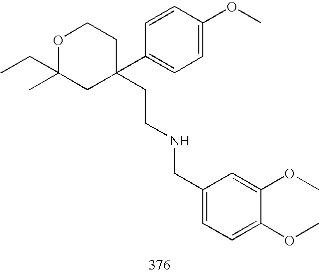 |
| 379 | 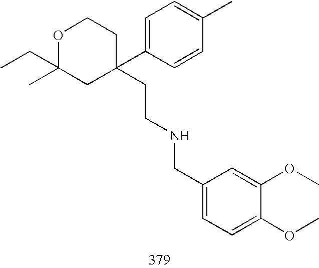 |
| 380 | 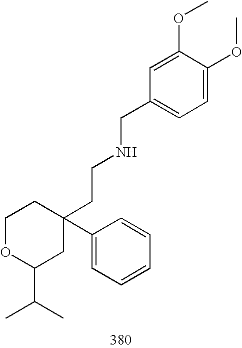 |
| 381 | 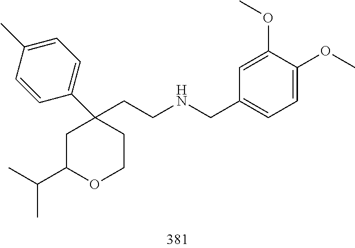 |
| 382 | 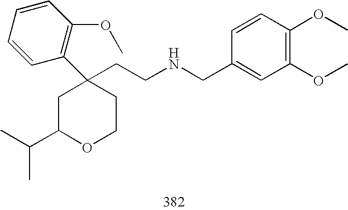 |
| 385 | 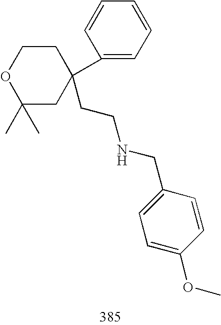 |
| 388 | 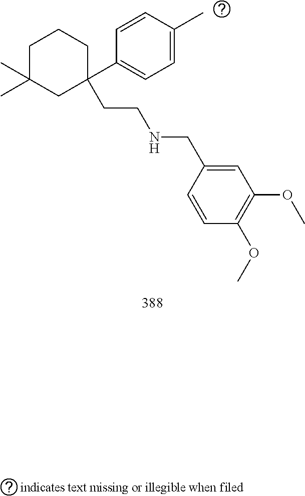 |
| 164-N | 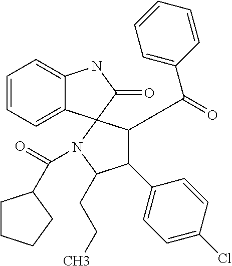 |
| 164-O | 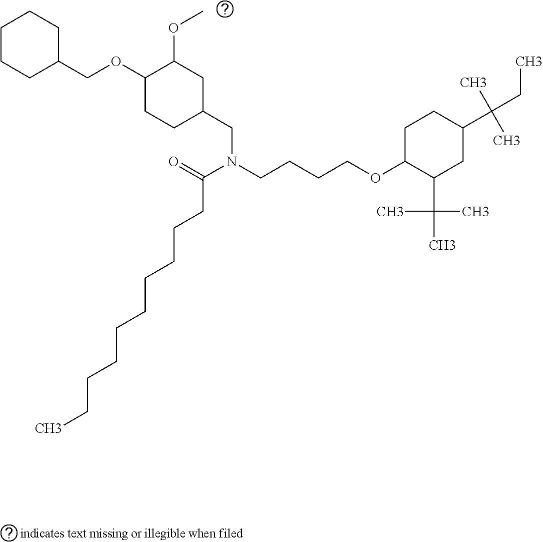 |
| 164-P | 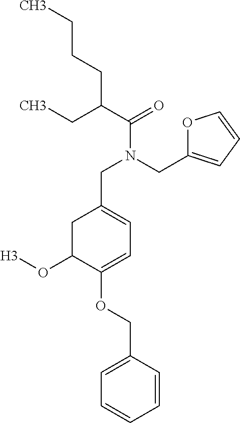 |
| 164-Q | 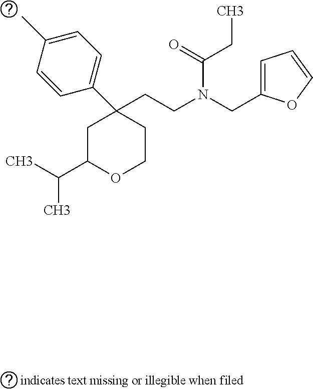 |
| 164-R | 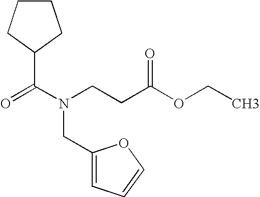 |
| 164-S | 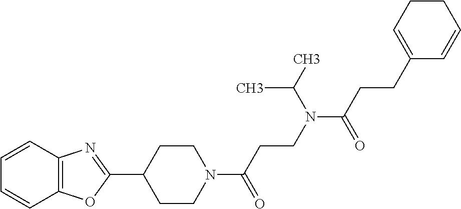 |

and pharmaceutically acceptable salts thereof.

In one embodiment, the compound or pharmaceutically acceptable salt of the compound is synthesized using combinatorial methodology.

In one embodiment, the compound or pharmaceutically acceptable salt of the compound is derivatized with a label.

In one embodiment, the label comprises biotin.

In one embodiment, the label comprises a fluorescent moiety.

In one embodiment, the label comprises fluorescein.

In one embodiment, the label comprises benzophenone.

The above compounds and pharmaceutically acceptable salts thereof are also useful in the methods of the present invention.

### Methods of Making Compounds

The compounds and pharmaceutically acceptable salts of compounds of the present invention can be prepared using a variety of methods starting from commercially available compounds, known compounds, or compounds prepared by known methods. General synthetic routes applicable to many of the compounds of the invention are included in the following scheme. It is understood by those skilled in the art that protection and deprotection steps not shown in the Scheme may be required for these syntheses, and that the order of steps may be changed to accommodate functionality in the target molecule.

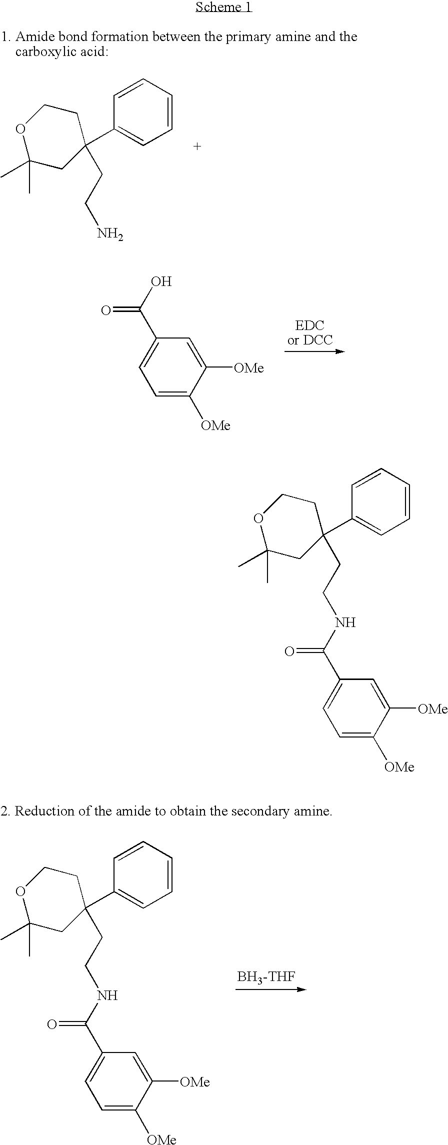

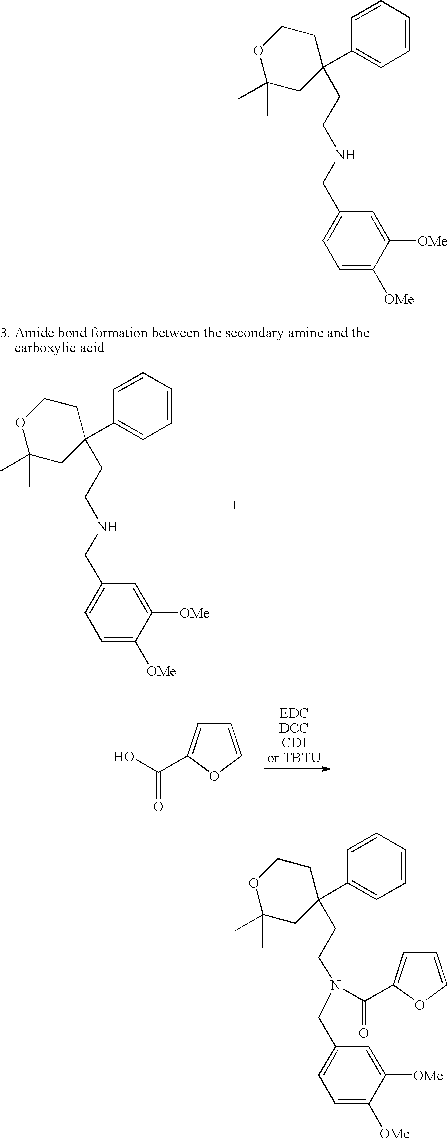

An exemplary synthetic scheme is shown in Scheme 1. In Step 1 of Scheme 1, an amide bond is formed between a primary amine and a carboxylic acid. The coupling reaction can be carried out, for example, in the presence of a coupling reagent such as 1-[3-(dimethylamino)propyl]-3-ethylcarbodiimide hydrochloride or 1,3-dicyclohexylcarbodiimide, in a solvent such as dichloromethane or chloroform. Step 2 demonstrates the subsequent reduction of the amide to obtain a secondary amine. The reduction can be carried out, for example, in the presence of BH3-THF in a solvent such as THF. Formation of the amide bond can be achieved as demonstrated in Step 3, wherein the secondary amine is coupled to an appropriate carboxylic acid in the presence of a coupling agent such as EDC, DCC, 1,1′-carbonyldiimidazole or O-benzotriazol-1-yl-N,N,N′,N′-tetramethyluronium tetrafluoroborate, and in a solvent such as chloroform, dichloromethane or acetonitrile.

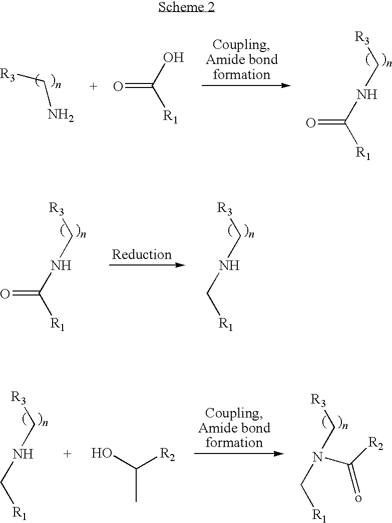

Scheme 2 demonstrates the general synthesis of a compound of Formula (II), wherein m is 1. First, an amide bond is formed between a primary amine and carboxylic acid, in the presence of a coupling reagent such as 1-[3-(dimethylamino)propyl]-3-ethylcarbodiimide hydrochloride or 1,3-dicyclohexylcarbodiimide, in a solvent such as dichloromethane or chloroform. The amide is subsequently reduced to produce the secondary amine, for example, in the presence of BH₃-THF in a solvent such as THF. Finally, the secondary amine amine is coupled to an appropriate carboxylic acid in the presence of a coupling agent such as EDC, DCC, 1,140 -carbonyldiimidazole or O-benzotriazol-1-yl-N,N,N′,N′-tetramethyluronium tetrafluoroborate, and in a solvent such as chloroform, dichloromethane or acetonitrile.

One of skill in the art will recognize that the synthetic methodology of Scheme 2 is also applicable to the other compounds of the invention.

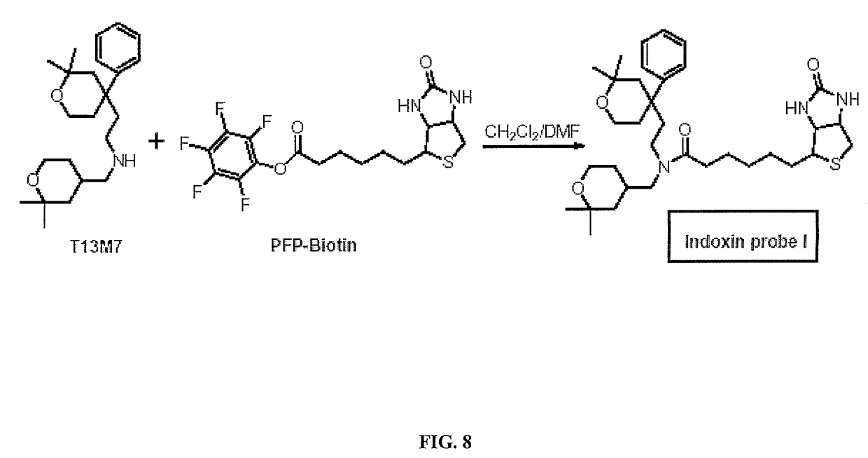

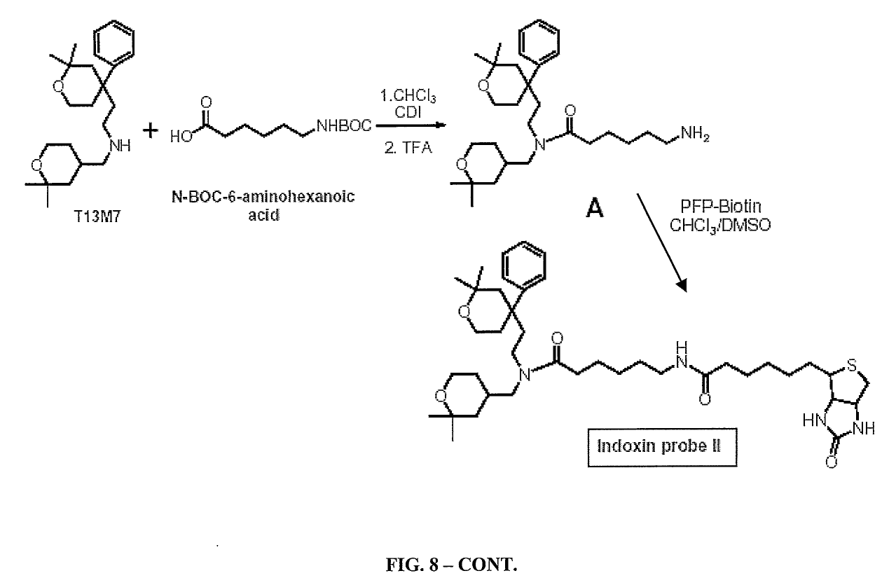

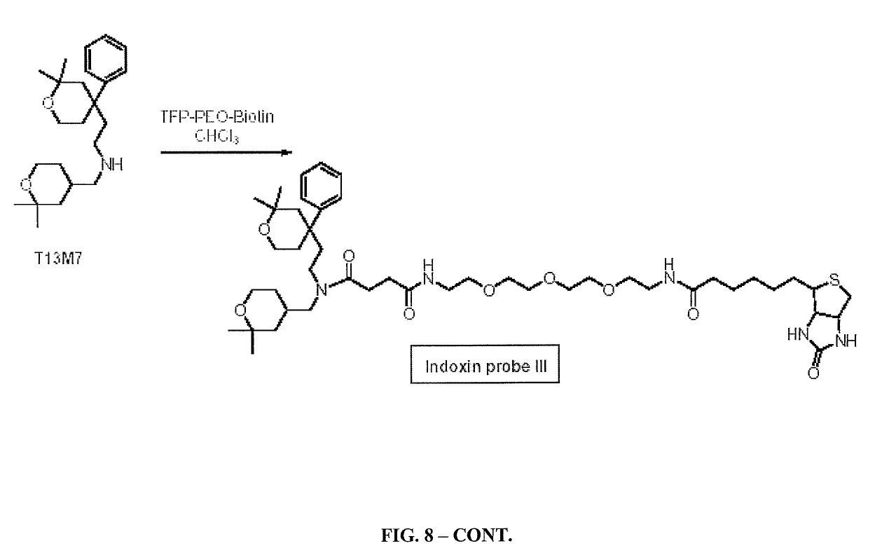

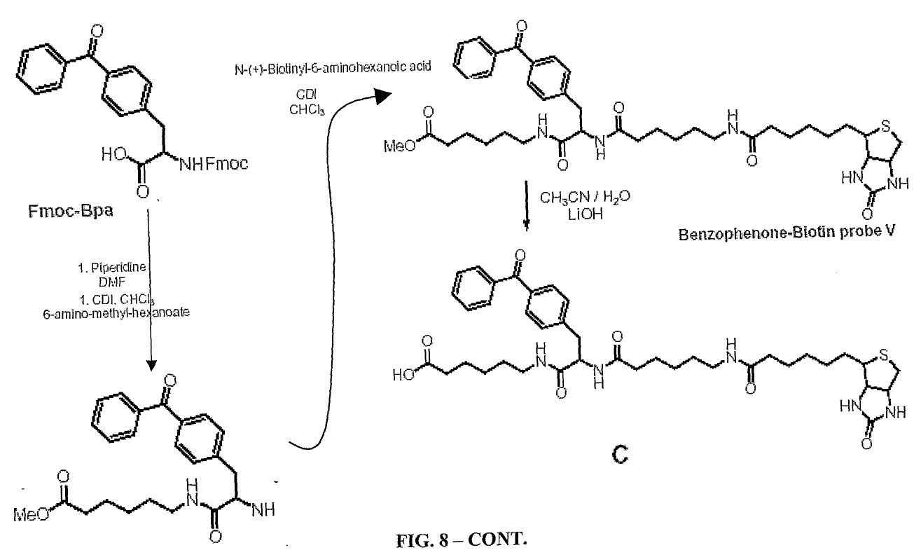

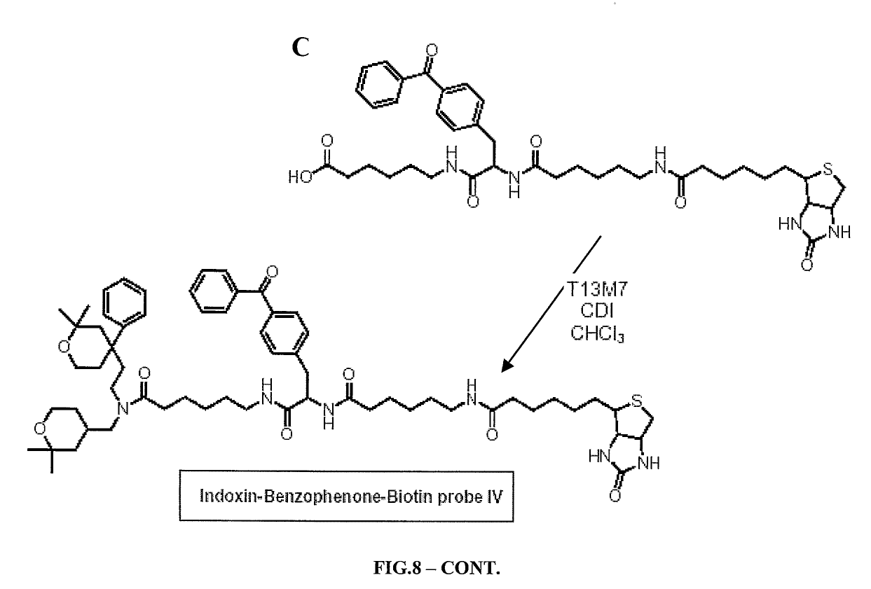

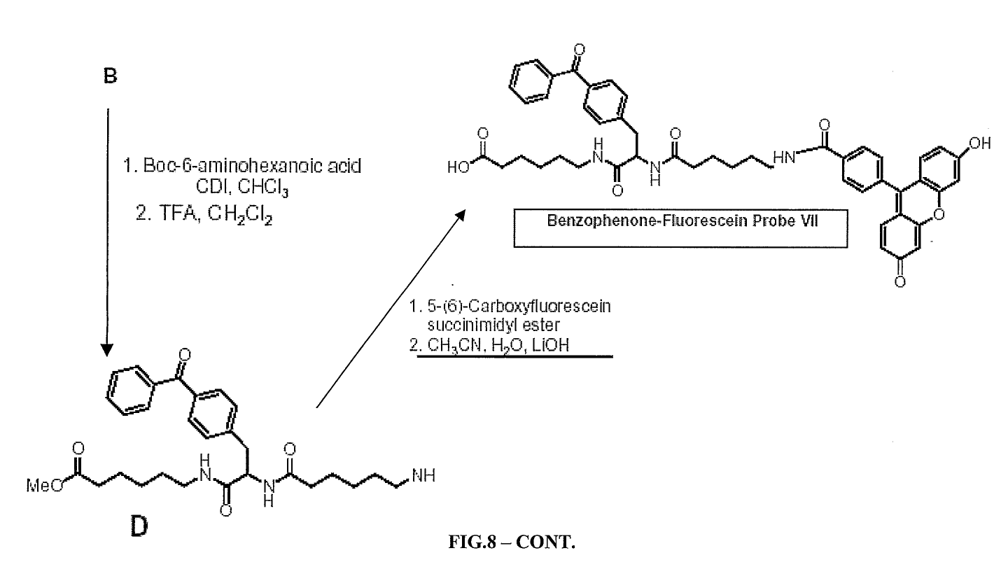

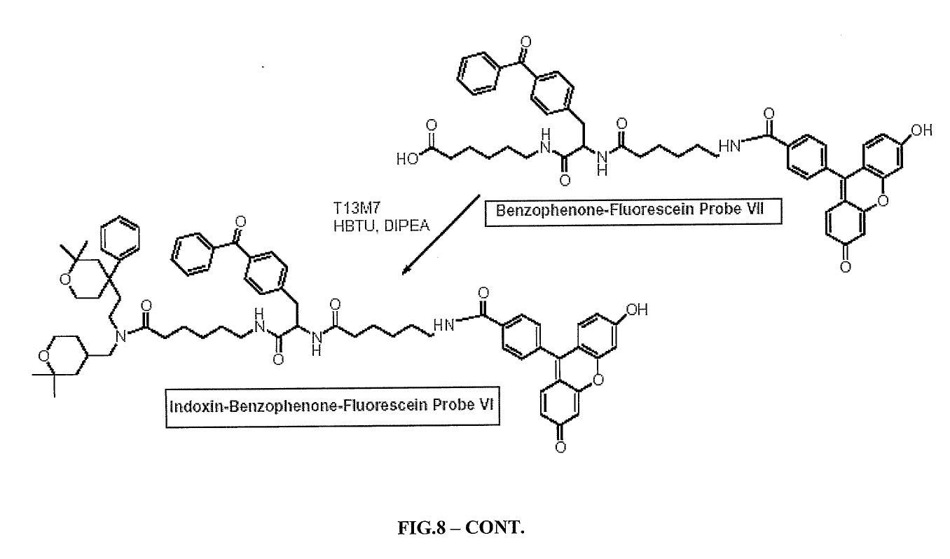

Additional methods for synthesizing compounds useful in the present invention are outlined, e.g., in FIG. 8.

### Methods of Identifying Targets of Compounds

In one embodiment, the invention provides a method for identifying cellular components involved in tumorigenesis. In the method of the present invention, (a) a tumorigenic cell (such as an engineered human tumorigenic cell), is contacted with an apoptosis-inducing drug (such as doxorubicin) and with indoxin; and (b) cellular components that interact (directly or indirectly) with the indoxin are identified, whereby cellular components may be involved in tumorigenesis. The cell can be contacted with indoxin and the apoptosis-inducing drug simultaneously or sequentially. Cellular components that interact with indoxin, or any compound of the present invention, may be identified by known methods understood by one skilled in the art.

In another embodiment, the invention provides a method to identify cellular components involved in HD, whereby a cell having htt-induced toxicity, such as an engineered neuronal cell, is contacted with an anti-HD test compound (for example, secondary acylated amines, such as revertins); and after contact, cellular components that interact (directly or indirectly) with the anti-HD test compound are identified, resulting in identification of cellular components involved in HD. Cellular components that interact with “revertin,” or any compound of the present invention, may be identified by known methods understood by one skilled in the art.

In a further embodiment, the subject compounds identified from screening a combinatorial library (such as indoxin) can be derivatized with another compound. One advantage of this modification is that derivatizing the compound can be used to facilitate the collection of a compound-target-complex. Derivatizing the compound can also be used to facilitate its collection, especially after the compound and target are separated from one another. Non-limiting examples of derivatizing groups used in the art include glutathione S transferase (GST), biotin, protein A, green fluorescent protein (GFP), GFP-derivatives, photoactivatible crosslinkers, fluorescein, digoxygenin, isotopes, polyhistidine, magnetic beads, or any combinations thereof.

The interaction between the compound and its target may be covalent or non-covalent. The compound of the compound-target pair also may or may not display affinity for other targets thus is target specific. In one embodiment of the invention, binding between a compound and its target can be identified at the protein level using in vitro biochemical methods established in the art. Some non-limiting examples for detecting such binding events include radiolabeled ligand binding, affinity chromatography, and photo-crosslinking (Jakoby W B, et al, Methods in Enzymology 1974, 46:1). Alternatively, small molecules can be immobilized on an agarose matrix and used to screen extracts of organisms and a variety of cell types.

In some embodiments of the invention, the cellular targets may be screened in a mechanism-based assay, such as a binding assay to detect compounds that bind to the target. One skilled in the art understands that this may include a fluid phase or solid phase binding event with the compound, the protein, or a reporter of either being detected. Non-limiting examples of binding reaction conditions may include the compound incorporating a marker such as glutathione S transferase (GST), biotin, protein A, green fluorescent protein (GFP), GFP-derivatives, photoactivatible crosslinkers, fluorescein, digoxygenin, isotopes, polyhistidine, magnetic beads, or any combinations thereof. Alternatively, a gene encoding a protein with previously undefined function that was identified by said binding of the compound to the target can be transfected with a reporter system known to those skilled in the art (for example, luciferase, β-galactosidase, or GFP) into a cell, and screened against the library; either by a high throughput screening or with individual members of the compound library.

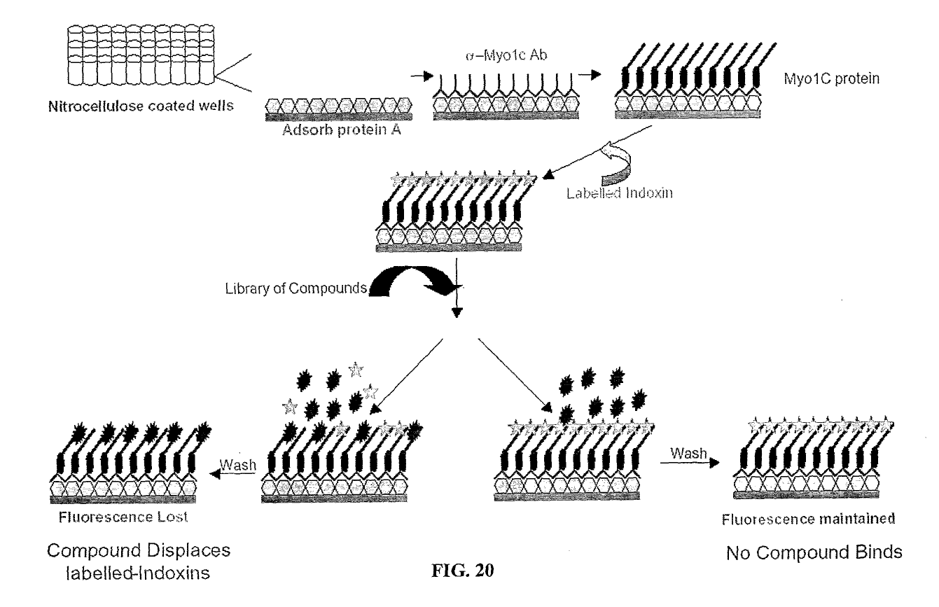

Those skilled in the art may also employ other mechanism-based binding assays. Some methods include, but are not limited to, biochemical assays which measure an effect on enzymatic activity, binding assays which detect changes in free energy, and cell-based assays in which the target and a reporter system (for example, luciferase or β-galactosidase) have been introduced into a cell. Binding assays can be performed with the target fixed to a well, bead, or chip; or captured by an immobilized antibody; or resolved by capillary electrophoresis. In one embodiment, the target (for example, Myo1c) can be adhered to a coated surface (for example, a protein A coated well or an actin-coated well, as depicted in FIG. 20). In other embodiments, the labeled compound (for example, a fluorescein-conjugated indoxin) can be added to the target protein-coated wells. In another embodiment, the target protein-coated wells bound with the labeled-compound can be exposed to a library of compounds, wherein binding of the new, unlabeled compound can displace the labeled compound. The newly bound compounds may be detected usually using calorimetric or fluorescence detection, or surface plasmon resonance, wherein displacement of a labeled compound, such as a fluorescein-conjugated indoxin, would result in a decrease in fluorescence intensity.

### Methods of Treatment

The present invention further presents methods of treating and/or preventing a disease, such as, cancer or HD, by modulating the function of a target (cellular component) that is identified according to the invention. In some embodiments, the function of the cellular component can be modified by altering the activity or expression of the target protein. According to the invention, if a target is identified to promote tumor cell proliferation, a therapeutic agent can be used to inhibit or reduce the function (activity or expression) of the target. If a target is identified to inhibit tumor growth, a therapeutic agent can be used to enhance the function (activity or expression) of the target. Alternatively, if a target is identified to increase tumor cell lethality, a therapeutic agent can be used to enhance the function (activity or expression) of the target. The therapeutic agents can include, but are not limited to: small molecules, proteins, antibodies, or nucleic acids (such as, an antisense oligonucleotide or a small inhibitory RNA for RNA interference).

In some embodiments, the invention provides a method for treating or preventing a neurodegenerative disorder associated with polyglutamine (polyQ) expansion in an individual. This method comprises administering to the individual a therapeutically effective amount of an agent identified by the methods of the invention as described above. Some non-limiting examples of neurodegenerative disorders associated with polyQ expansion include, Huntington's disease, spinobulbar muscular atrophy, dentatorubral pallidoluysian atrophy, and the spinocerebellar ataxias type 1, 2, 3, 6, 7, and 17.

A cancer, tumor, or neoplasia is characterized by one or more of the following properties: Loss of anchorage dependence; cell growth is not regulated by the normal biochemical and physical influences in the environment (such as failure to respond to cell cycle checkpoints and increasing resistance to apoptosis); Anaplasia (e.g., lack of normal coordinated cell differentiation); and in some instances, metastasis.

In certain embodiments, the invention provides for a method of treating or preventing cancer disease in a subject (for example, a human). Non-limiting examples of cancer diseases include: bladder carcinoma, breast carcinoma, anal carcinoma, cervix carcinoma, chronic myelogenous leukemia, chronic lymphocytic leukemia, hairy cell leukemia, endometrial carcinoma, lung (small cell) carcinoma, head and neck carcinoma, multiple myeloma, follicular lymphoma, ovarian carcinoma, non-Hodgkin's lymphoma, colorectal carcinoma, brain tumors, Kaposi's sarcoma, hepatocellular carcinoma, lung (non-small cell carcinoma), pancreatic carcinoma, prostate carcinoma, melanoma, soft tissue sarcoma, and renal cell carcinoma. Additional cancer disorders can also be found in Isselbacher et al. (1994) Harrison's Principles of Internal Medicine, 1814-1877; and Ruddon R W, Cancer Biology, 3^{rd} Ed., 1995. Oxford University Press, which are herein incorporated by reference.

In one embodiment of the present invention, it relates to a method of treating or preventing cancer in a subject. The method comprises administering to the subject (for example a mouse or human) a therapeutically effective amount of a compound that is selectively toxic to an engineered human tumorigenic cell (for example, human colon cancer cells) in addition to an anti-tumor agent. In other embodiments, the anti-tumor agent used can be an anthracycline (such as doxorubicin). In some embodiments, the cancer is characterized by cells wherein an E6/p53 mediated pathway is activated. In further embodiments, the cancer is characterized by engineered cells expressing the E6 oncoprotein.

A number of conventional compounds have been shown to have anti-tumor activities, and thus have been used as pharmaceutical agents in chemotherapy to shrink solid tumors, prevent further tumor growth and metastases, or reduce the number of malignant cells in bone marrow malignancies or leukemia. Although chemotherapeutic compounds have been effective in treating various types of cancer disease, many anti-tumor compounds cause undesirable side effects. In many instances, when two or more different anti-cancer treatments are combined, the treatments can work synergistically. This subsequently allows for dosage reduction of each of the treatments, thereby reducing the unfavorable side effects induced by each compound at higher doses.

In one embodiment, pharmaceutical compositions of the present invention may be administered in combination with a conventional anti-tumor compound. Non-limiting examples of conventional anti-tumor compounds include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfuner, mesna, pamidronate, streptozocin, teniposide, procarbazine, titanocene dichloride,raltitrexed, rituximab, testosterone, thioguanine, vincristine, vindesine, thiotepa, topotecan, tretinoin, vinblastine, trastuzumab, and vinorelbine.

Those skilled in the art understand which characteristics are used to determine if a cancer, tumor, or neoplasia has been treated and are responding to chemotherapy. One skilled in the art, for example, may assess for a decrease in the number of tumor cells, a decrease in tumor size, or a decrease in cell proliferation. It is recognized that the treatment of the present invention may be a lasting and complete response or can encompass a transient or partial clinical response. See, for example, Isselbacher et al. Harrison's Principles of Internal Medicine 13^{th} Ed, 1996, 1814-1882, and Ruddon R W, Cancer Biology, 3^{rd} Ed., 1995. Oxford University Press, which are herein incorporated by reference.

Assays to determine the sensitization or the enhanced death of tumor cells are well known in the art. Some non-limiting examples include: assays for morphological signs of cell death; agarose gel electrophoresis of DNA extractions or flow cytometry to determine DNA fragmentation, a characteristic of cell death; standard dose response assays that assess cell viability; chromatin assays (e.g., counting the frequency of condensed nuclear chromatin); assays that measure the activity of polypeptides involved in apoptosis; and drug resistance assays as described in Lowe S W, et al, Cell 1993, 74: 957-697, and in U.S. Pat. No. 5,821,072, which are herein incorporated by reference.

### Therapeutic Administration

When administered to an animal, the compounds or pharmaceutically acceptable salts of the compounds of the invention can be administered neat or as a component of a composition that comprises a physiologically acceptable carrier or vehicle. A composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, exipient, or diluent.

The present compositions, comprising compounds or pharmaceutically acceptable salts of the compounds of the invention can be administered orally. The compounds or pharmaceutically acceptable salts of compounds of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound or a pharmaceutically acceptable salt of the compound into the bloodstream. The mode of administration is left to the discretion of the practitioner.

In one embodiment, the compound or a pharmaceutically acceptable salt of the compound is administered orally.

In another embodiment, the compound or a pharmaceutically acceptable salt of the compound is administered intravenously.

In another embodiment, it may be desirable to administer the compound or a pharmaceutically acceptable salt of the compound locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compound or a pharmaceutically acceptable salt of the compound into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound or a pharmaceutically acceptable salt of the compound can be formulated as a suppository, with traditional binders and excipients such as triglycerides.

In another embodiment, the compound or a pharmaceutically acceptable salt of the compound can be delivered in a vesicle, in particular a liposome (see Langer, Science 249:1527-1533 (1990) and Treat et al., Liposomes in the Therapy of Infectious Disease and Cancer 317-327 and 353-365 (1989)).

In yet another embodiment, the compound or a pharmaceutically acceptable salt of the compound can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249 :1527-1533 (1990); Sefton, CRC Crit. Ref. Biomed. Eng. 14:201 (1987); Buchwald et al., Surgery 88:507 (1980); and Saudek et al. N. Engl. J Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the compound or a pharmaceutically acceptable salt of the compound, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to an animal. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The compound or pharmaceutically acceptable salt of the compound of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and prenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences 1447-1676 (Alfonso R. Gennaro, ed., 19th ed. 1995).

In one embodiment, the compound or a pharmaceutically acceptable salt of the compound is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of the compounds or pharmaceutically acceptable salts of the compounds with inert fillers and/or diluents such as pharmaceutically acceptable starches ( e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment, the compound or a pharmaceutically acceptable salt of the compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the compound or a pharmaceutically acceptable salt of the compound is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In another embodiment, the compound or pharmaceutically acceptable salt of the compound can be administered transdermally through the use of a transdermal patch. Transdermal administrations include administrations across the surface of the body and the inner linings of the bodily passages including epithelial and mucosal tissues. Such administrations can be carried out using the present compounds or pharmaceutically acceptable salts of the compounds, in lotions, creams, foams, patches, suspensions, solutions, and suppositories (e.g., rectal or vaginal).

Transdermal administration can be accomplished through the use of a transdermal patch containing the compound or pharmaceutically acceptable salt of the compound and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The compounds or pharmaceutically acceptable salts of the compounds of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound or a pharmaceutically acceptable salt of the compound can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the compound or a pharmaceutically acceptable salt of the compound to treat or prevent a cancer, the presence or development of tumors, or other conditions characterized by excessive cell proliferation, or neurodegenerative disorders in a minimal amount of time. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, the present invention is directed to prodrugs of the compounds or pharmaceutically acceptable salts of compounds of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

The amount of the compound or a pharmaceutically acceptable salt of the compound is an amount that is effective for treating or preventing a cancer, the presence or development of tumors, or other conditions characterized by excessive cell proliferation, or neurodegenerative disorders. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The compound or a pharmaceutically acceptable salt of the compound can be assayed in vitro or in vivo for the desired therapeutic or prophylactic activity prior to use in humans. Animal model systems can be used to demonstrate safety and efficacy.

The present methods for treating or preventing cancer, the presence or development of tumors, or other conditions characterized by excessive cell proliferation, or neurodegenerative disorders, can further comprise administering another therapeutic agent to the animal being administered the compound or a pharmaceutically acceptable salt of the compound. In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. The compound or a pharmaceutically acceptable salt of the compound and the other therapeutic agent can act additively or, in one embodiment, synergistically. In one embodiment of the invention, where another therapeutic agent is administered to an animal, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically.

### Example 1

Small molecule screening was employed to identify compounds and mechanisms for overcoming E6-oncoprotein-mediated drug resistance. For this purpose, a cell-based model with a defined genetic alteration was selected: expression of the E6 oncogene. Small molecules are capable of altering the function of gene products in a conditional manner. Since the use of small molecules as therapeutics has been well established (compared to siRNA or cDNA), it is more likely that they can serve as leads for the development of new drug candidates. Synthetic compounds are also less prone to degradation via catabolic pathways and are more likely to be cell permeable and maintain biological activity in vivo. Using high-throughput screening in isogenic cell lines, compounds that potentiate doxorubicin's lethality in E6-expressing colon cancer cells were identified. Such compounds included quaternary ammonium salts, protein-synthesis inhibitors, 11-deoxyprostaglandins, analogs of 1,3-bis(4-morpholinylmethyl)-2-imidazolidinethione (a thiourea), and acylated secondary amines, referred to herein as indoxins. Indoxins upregulated topoisomerase IIα, the target of doxorubicin, thereby increasing doxorubicin lethality. A photolabeling strategy was developed to identify targets of indoxin and a nuclear actin-related protein complex was identified as a candidate indoxin target.

### Methods

### Cell Lines

RKO colon carcinoma, human (ATCC, order # CRL-2577), RKO-E6 colon carcinoma transfected with HPV E6 inserted in pCMV.3 (ATCC, order # CRL-2578) were grown in MEM with 2 mM L-glutamine and Earle's BBS adjusted to contain 1.5 g/L NaHCO₃, 0.1 mM non-essential amino acids and 1.0 mM sodium pyruvate, and supplemented with 10% fetal bovine serum, penicillin and streptomycin (pen/strep). Both cell lines were incubated at 37° C. in a humidified incubator containing 5% CO₂.

### Compound Libraries

An annotated compound library (ACL) comprising 2,083 compounds, an NCI diversity set of 1,990 compounds obtained from the National Cancer Institute, and a combinatorial library (also referred to as a TIC library herein) comprising 12,960 synthetic compounds (Chembridge) and 10,725 natural products and their analogs (IBS and TimTec) were used in the primary screen. All compound libraries were prepared as 4 mg/ml solutions in DMSO in 384-well polypropylene plates and stored as −20° C.

Benzalkonium chloride (cat #234427, MW), benzethonium chloride (cat #B-8879, MW 448.1), D-biotin (cat #B-4501, MW 244.3), S-(+)-camptothecin (cat #C9911, 348.4), cetyltrimethylammonium bromide (cat #855820, MW 364.46), cycloheximide (C-76798, MW 281.4), doxorubicin (cat #D-1515, MW 580.0), hexadecylpyridinium chloride monohydrate (cat #C9002, MW 358.01), hydroxyurea (cat #H-8627, MW 76.05), and podophyllotoxin (cat #P-4405, MW 414.4) were obtained from Sigma-Aldrich. Indoxin A (MW 415.61) and indoxin B (MW 423.59) were obtained from Interbioscreen Ltd.

### Compound Designation

Each hit compound from the primary screen was assigned a designation based on the location of this compound in the libraries. For example, the designation T86N7 indicates that compound is located on the TIC library's mother plate number 86, row N, column 7.nnnn

### Screening

Daughter replica plates were prepared with a Zymark Sciclone ALH by diluting DMSO stock plates 50-fold in medium lacking serum and penicillin/streptomycin to obtain a compound concentration in daughter plates of 80 μg/ml with 2% DMSO. Assay plates were prepared by seeding cells in black, clear bottom 384-well pates (Coming inc., cat #3712). Columns 3-22 were treated with compounds from a daughter library plate by transferring 3 μl from the daughter library plate using 384-position fixed cannula array. The final compound concentrations in assay plates were 4 μg/ml.

### Primary Screen: Alamar Blue Viability Assay

The Alamar Blue assay incorporates a fluorimetric/colorimetric growth indicator that changes color in the response to chemical reduction by viable cells (Nociari 1998). Cells were seeded at a density of 3,000 cells (57 μl) per well in a 384-well black, clear-bottom plates using a syringe bulk dispenser (Zymark Sciclone ALH). Three microliters were removed from a compound daughter plate using a 384 fixed cannula head and added to the assay plate, making the final concentration 4 μg/ml. The plates were incubated for 24 h at 37° C. 10 μl of 40% Alamar Blue (Biosource Int., cat #DAL1100) solution in media was added to each well (1:10 dilution). The assay plates were incubated for 16h. Fluorescence intensity was determined using a Packard Fusion platereader with a 535 nm excitation filter and a 590 nm emission filter. The average fluorescence for the whole plate was used as a positive control. Average percentage inhibition for each well was determined using our freely available SLIMS software (Kelley et al., 2004).

### Retesting of Compounds in a Dilution Series

The daughter plates for the 2-fold dilution series were prepared with a Zymark Sciclone ALH by diluting the replica daughter plates to obtain compound concentrations from 8 μg/ml-0.008 μg/ml (columns 8-18). Columns 1-7 were used as untreated control. RKO-E6 cells were seeded at the density of 3,000 per well in 55 μl, and 5 μl were added from the daughter plate. RKO cells were seeded at the density of 2,000 per well in 55 μl, and 5 μl were added from the daughter plate. The assay was incubated for 24 h at 37° C. 10% Alamar Blue was added to each well and the assay was incubated for 16 h (see above). Compounds for retesting were purchased from the manufacturers or from Sigma-Aldrich (see above). Stocks were prepared in DMSO at 4 mg/ml.

### Bliss Independence Analysis

Each cell line (RKO, RKO-E6, HeLa and TC32) was treated with a dose combination matrix of doxorubicin and a selected hit compound. Each treatment was done in a triplicate in 384-well format. The average percent inhibition of cell proliferation, measured with Alamar Blue, was determined using the following formula:

Percent inhibition=(1−(X−N)/(P−N))

X—the average fluorescence readout for each treatment

N—the negative control signal (in the absence of any cells)

P—the positive control (the average fluorescence readout of untreated cells)

The predicted Bliss additive effect was determined using the following formula:

C=A+B−A·B

C—the combined response for two single compounds with effects A and B

A—the percent inhibition of compound A at the particular concentration

B—the percent inhibition of compound B at the particular concentration

The excess over predicted Bliss independence was calculated by subtracting the predicted Bliss effect for each treatment from the experimentally determined percent inhibition for the same treatment.

### Western Blot Analysis

Antibodies were obtained form the following suppliers: p53 (Oncogene/Calbiochem, cat #OP43), p21 (Santa Cruz Biotechnology inc., cat #sc-817), MDM2 (Santa Cruz Biotechnology Inc., cat #sc-965), Topoisomerase IIα (TopoGEN, cat #2011-1), Topoisomerase I (BD PharMingen, cat #556597), Cyclin B (BD Transduction Laboratories, cat #610219), Cyclin A (BD Transduction Laboratories, cat #611268), Anti-Fluorescein-HRP (Molecular Probes, cat #A21253), Anti-biotin-HRP (Cell Signaling Technology, cat #7075, Jackson ImmunoResearch Laboratories, cat #200-032-096), eIF4E (Santa Cruz Biotechnology Inc., cat #sc-9976 HRP).

Cells were lysed in denaturing lysis buffer (50 mM HEPES KOH [pH 7.4], 40 mM NaCl, 2 mM EDTA, 1.5 mM Na₃VO₄, 50 mM NaF, 10 mM sodium pyrophosphate, 10 mM sodium β-glycerophosphate, 0.5% Triton X-100, and protease inhibitor tablet [Roche, cat #11777700]). Protein content was quantified using a BioRad protein assay reagent (BioRad, cat #500-0006). Equal amounts of protein were resolved on SDS-polyacrylamide gels. Proteins were transferred onto a PVDF membrane, blocked with 5% milk, and incubated with the appropriate primary and secondary antibodies. The membranes were developed with chemiluminescence reagent (5 ml of 100 mM TRIS buffer [pH 8.5] and 5 μl of 30% H₂O₂ were mixed with 10 ml of 100 mM TRIS buffer [pH 8.5], 11 μl of 90 mM p-coumaric acid, and 25 μl of 250 mM luminol and immediately added to the PVDF membrane for 1 minute). To test for equivalent loading in each lane, blots were stripped, blocked and probed with an anti-eIF-4E antibody.

### Flow Cytometry

RKO, RKO-E6 or HeLa cells were seeded in 20-cm dishes in 10 ml of growth medium. Cells were allowed to adhere and then treated with the selected compounds for 24 h. The treated cells were collected, washed 2× with PBS and counted. The cell pellet was re-suspended in 500 μl of ice cold PBS and 2-5 ml of cold 70% ethanol (−20° C.) were added. Cells were fixed for 1 h or overnight. 5-10 million cells were placed into a 15-ml conical tube and centrifuged at 1000 g. Cells were washed 2× with 1% calf serum in PBS. Cells were resuspended in 800 μl of 1% calf serum in PBS and 100 μl of Rnase (1 mg/ml) and 100 μl of propidium iodide (400 μg/ml) added. Cells were incubated at 37° C. for 30 min and then analyzed on flow cytometer.

### Synthesis of Affinity Probes

Abbreviations used in the schematic of the synthesis of indoxin probes (as depicted in FIG. 8) are as follows: Boc,—t-butoxycarbonyl; CDI,—N,N′-carbonyldiimidazole; DIPEA,—diisopropylethylamine; DMF,—dimethylformamide; DMSO,—dimethyl sulfoxide; Fmoc,—9-fluorenylmethoxycarbonyl; HBTU,—O-benzotriazol-1-yl-N,N,N′,N′-tetramethyluronium hexafluorephosphate; PFP,—pentafluorophenol; TFA,—trifluoroacetic acid; TFP,—tetrafluorophenol. Solvents were purchased from Aldrich and used without further purification. Common synthetic reagents were obtained from commercial sources and used without further purification.

Indoxin derivative I: T13M7 (10 mg, 1 eq.) was dissolved in methylene chloride and added to PFP-Biotin (12 mg, 1 eq., Pierce, cat #21218) solution in DMF. The reaction was stirred for 1 h at RT and for 30 min at 40° C. A yield of >95% was confirmed by LC-MS. Solvents and pentafluorophenol were removed under high vacuum and the crude product was used without further purification.

Indoxin derivative II: BOC-6-aminohexanoic acid (36 mg, 1.1 eq) was dissolved in chloroform and CDI (27 mg, 1.2 eq.) was added to the reaction solution. The reaction mixture was stirred at RT for 30 min, then T13M7 (50 mg, 1 eq.) added to the reaction. The coupling reaction proceeded slowly and required heating at 50° C. and addition of CDI. The BOC-protecting group was removed with 30% TFA/70% CH₂Cl₂ at RT for 30 minutes. Solvents and TFA were removed under high vacuum, and the crude product was extracted with CHCl₃/5% K₂CO₃ solution. Organic phase was separated, washed with dH₂O twice and dried under vacuum to provide the product A (FIG. 8). A (12 mg, 1 eq) was dissolved in chloroform and added to the PFP-Biotin solution in DMSO, followed by DIPEA (20 μl). The reaction was stirred at RT for 30 min. LC-MS analysis confirmed complete coupling. Chloroform, DIPEA and pentafluorophenol were removed under high vacuum and the crude product was used without further purification.

Indoxin derivative III: T13M7 (2.3 mg, 1 eq.) was dissolved in chloroform and added to TFP-PEO-Biotin (4.7 mg, 1 eq., Pierce, cat #21219) solution in chloroform. The reaction was stirred for 1 h at RT. LC-MS analysis indicated complete consumption of TFP-PEO-Biotin, ˜75% of product and ˜25% of T13M7 excess. The solvent and tetrafluorophenol were removed under high vacuum; the crude product was triturated with ethyl ether to remove the excess of T13M7 and dried under vacuum. The product was used without further purification.

Benzophenone-biotin: N-(+)-Biotinyl-6-aminohexanoic acid (239 mg) and 4-benzoyl-L-phenylalanyl-6-aminoxehanoic acid methyl ester (237 mg) were dissolved in DMF, 230 μl of DIPEA were added to the reaction mixture, followed by 237 mg of HBTU. The reaction mixture was stirred at RT for 30 min. LC-MS analysis showed complete reaction. The solvent and excess of DIPEA were removed under high vacuum. The crude product was dissolved in CHCl₃ and extracted 2× with 1% HCl. Organic phase was separated, solvent was removed and the product was purified using rotary chromatography (SiO₂ solid phase). Yield 325 mg (73.4%).

Indoxin-Benzophenone-Biotin IV:

1. Compound V (100 mg) was dissolved in aqueous methanol, 1 eq of LiOH was added to the reaction mixture and the reaction was refluxed overnight. LC-MS analysis confirmed quantitative hydrolysis of the methyl ester. The crude product was dissolved in CHCl₃ and extracted with 1% HCl and dH₂O. Organic phase was separated and dried over anhydrous Na₂SO₄ for 1 h. Then Na₂SO₄ was filter off and solvents were removed under vacuum to provide pure product in a quantitative yield.

2. N-[N-(+)-Biotinyl-6-aminohexanoyl]-4-benzoyl-L-phenylalanyl-6-aminoxehanoic acid (C) was dissolved in CHCl₃ and 1.1 eq. of CDI was added to the reaction mixture. The reaction was stirred at RT for 30 min and then T13M7 was added to the reaction. The reaction was stirred at RT for 1 h and at 50° C. for 30 min. LC-MS analysis confirmed complete coupling. The solution of the crude product in CHCl₃ was extracted with 0.1% HCl and dH₂O. The organic phase was separated and dried over anhydrous Na₂SO₄ for 1 h. Then Na₂SO₄ was filter off and solvents were removed under vacuum to provide more than 90% pure product; indoxin-benzophenone-biotin VI.

Benzophenone-Fluorescein VII:

1. BOC-6-aminohexanoic acid (105 mg) was dissolved in ˜5 ml of CHCl₃ and 1.1 eq. of CDI (74 mg) was added to the reaction. The reaction mixture was stirred under argon at RT for 30 min. Then 180 mg of 4-benzoyl-L-phenylalanyl-6-aminoxehanoic acid methyl ester (B) were added to the reaction and the reaction mixture was stirred at RT for 2 h. LC-MS analysis indicated complete coupling. The crude was extracted twice with H₂O (pH ˜3) and once with dH₂O to remove imidazole. The organic phase was separated and solvent was removed under vacuum. The oily product was dissolved in 3 ml of CH₂Cl₂ and 1 ml of TFA was added to the reaction. The reaction mixture was stirred at RT for 30 min. LC-MS and TLC analysis indicated complete removal of BOC-protecting group, which provided quantitative yield of compound D.

2. 50 mg of compound D were dissolved in ˜1 ml of DMF and added to the solution of 5-(and-6)-carboxyfluorescein succinimidyl ester (46.5 mg) in DMF. The reaction mixture was stirred at RT for 2 h and heated at 50° C. for 15 min. Solvents were removed and the crude product was used for the next step without further purification: the crude product was dissolved in CH₃CH/H₂O (1:1) and LiOH was added to the reaction mixture till pH 11. The reaction was refluxed for 1 h. LC-MS analysis indicated complete hydrolysis of methyl ester. Then solvents were removed under vacuum, the crude product was dissolved in CHCl₃/EtOH and extracted with acidic water (pH=1). The organic phase was separated and the crude product was purified using rotary TLC (SiO₂ solid phase) to provide benzophenone-fluorescein VII.

Indoxin-benzophenone-fluorescein VI: Benzophenone-fluorescein probe VII (6.8 mg) and T13M7 (2.86 mg) were dissolved in 1 ml of DMF. DIPEA and HBTU (2.8 mg) were added to the reaction mixture and the reaction mixture was stirred at RT for 30 min. LC-MS analysis indicated only ˜50% coupling, therefore, another 2.8 mg of HBTU were added and the reaction mixture was heated at 50° C. for 10 min. LC-MS analysis confirmed more than 90% coupling reaction. DMF was removed under high vacuum to provide crude product. The oily residue was triturated with Et₂O to remove excess of T13M7, DIPEA and HBTU by-products. Then the product; indoxin-benzophenone-fluorescein VI was used without further purification.

### Affinity Purification of Protein Targets

Biotinylated Indoxin probes: RKO-E6 cells were lysed in the non-denaturing lysis buffer (1% NP-40, 50 mM Tris-HCl, pH 7.4, 300 mM NaCl, 5 mM EDTA, 0.02% sodium azide, 10 mM iodoacetamide, 1 mM PMSF, and protease inhibitor tablet (Roche, cat #11777700)). The cell lysates were pre-cleared with Immobilized NeutrAvidin gel (Pierce Biotechnology, cat #29200) for 1 h at 4° C. Then the cell lysates were incubated with the small molecule affinity probes for 10-12 h at 4° C. NeutrAvidin beads were added to the cell lysates incubated with the indoxin probes I, II, III comprised of indoxin moiety, linker and biotin, and incubated at 4° C. for 1 hr. The NeutrAvidin beads were separated from the cell lysates and washed twice with the non-denaturing lysis buffer and twice with PBS (for 1D analysis) or with dH₂O (for 2D analysis). The samples for 1D analysis were boiled in SDS page buffer and resolved using Tris-Glycine pre-cast gels. Proteins were detected with coomassie blue or silver stain. The samples for 2D analysis were sonicated in ZOOM 2D Protein Solubilizer 1 (Invitrogen, cat #ZS10001) and resolved following the protocol provided by Invitrogen. The cell lysates with the photo-reactive probes IV and V were placed in the optical glass cells (Starna, cat #1-SOG-10-GL14-S), purged with argon gas for 5 min and irradiated at 350 nm for 15 min in Rayonet Reactor. The cross-linked proteins were isolated and resolved as described above. Western blot analysis was done using anti-biotin antibody.

Fluoresceinylated Indoxin probes: The above described protocol was used to isolate proteins cross-linked to photo-reactive probes VI and VII using total cell lysates. The nuclear and cytosolic fractions were separated following the published protocol (Lee et al., 1988; Lee and Green, 1990), except, the cytoplasmic and the nuclear fractions were used for the affinity purification without dialysis. The affinity purification was done as described above, except, Protein A beads and anti-Fluorescein antibody were used for the pull-down of cross-linked proteins.

### Results

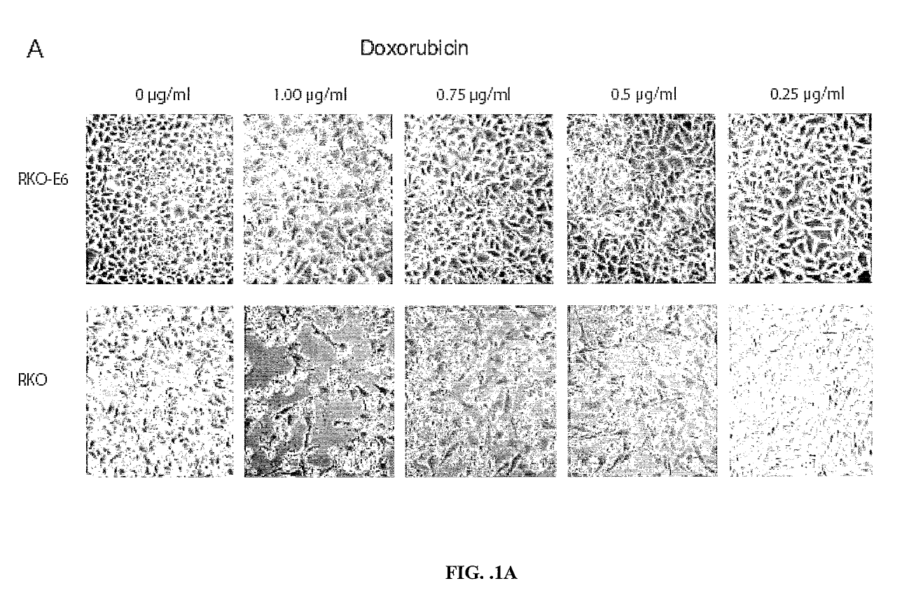

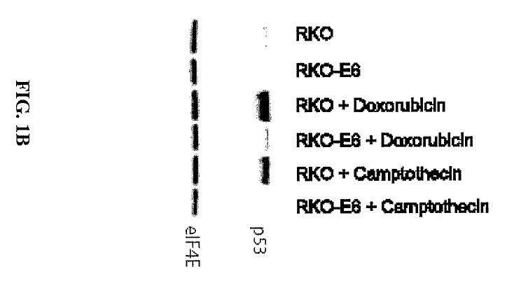

A cell-based model, the RKO colon carcinoma cell line (with high levels of p53) and an isogenic RKO-E6 cell line expressing the E6 oncoprotein were used to identify compounds that overcome E6-induced drug resistance. RKO-E6 cells have lower levels of p53 and fail to arrest in G1/S phase after various drug treatments (Slebos et al., 1995). RKO-E6 cells were found to be two-fold to four-fold more resistant to the anticancer drug doxorubicin compared to RKO cells (FIG. 1). Doxorubicin is a DNA topoisomerase II-based DNA-damaging agent that induces double strand DNA breaks, p53 stabilization and apoptosis. Doxorubicin-treated RKO-E6 cells have lower levels of p53 than doxorubicin-treated RKO cells (FIG. 1), suggesting that resistance to doxorubicin is caused, at least in part, by E6-induced degradation of p53. Thus, compounds that overcome E6-induced resistance should restore doxorubicin's lethality in RKO-E6 cells. Therefore, such compounds might target E6, E6AP, p53, MDM2 or other proteins involved in the cellular response to doxorubicin.

In the primary screen, compounds were identified that inhibited proliferation of RKO-E6 cells in the presence of a dose of doxorubicin that they were otherwise resistant to. Cell viability was measured in 384-well format using Alamar Blue (Nociari et al., 1998), which is a fluorescent dye that detects cellular metabolic activity, and hence cellular viability. RKO-E6 cells were treated with 4 μg/ml of each test compound, corresponding to 10 μM for a compound with a molecular weight of 400, and 0.5 μg/ml (0.9 μM) of doxorubicin for 24 h. Each compound was tested in three replicates. Tested compounds were derived from an annotated compound library comprising 2,083 compounds (Root et al., 2003), the NINDS library of 1,040 compounds (Lunn et al., 2004), and a TIC library (Kelley et al., 2004), which encompasses 23,760 natural products, including both natural product analogs and synthetic compounds (all tested compound structures and all activity data are provided in the supplementary materials). 208 compounds were identified from the TIC library that induced at least 25% inhibition of cell in RKO-E6 cells in the presence of 0.5 μg/ml of doxorubicin, and 70 compounds from ACL and NINDS libraries that induced at least 30% RKO-E6 cell growth inhibition in the presence of 0.5 μg/ml of doxorubicin. It was reasoned that among these 278 compounds there should be compounds that are not lethal on their own but restore sensitivity of RKO-E6 cells to doxorubicin.

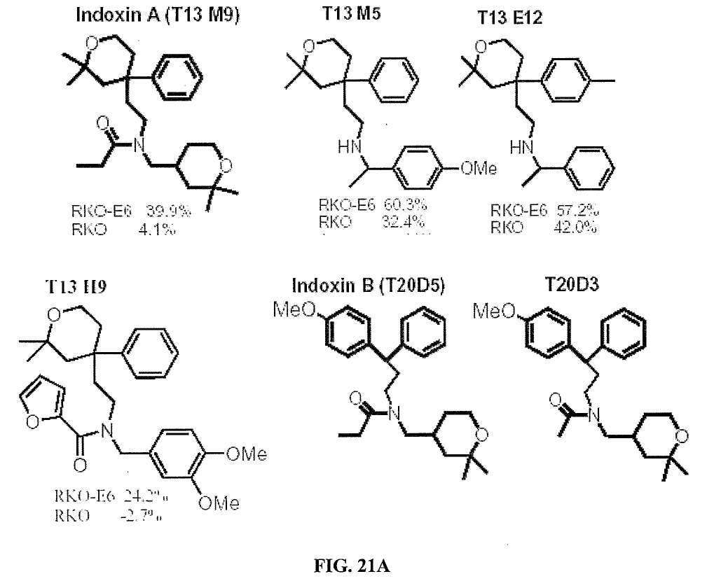

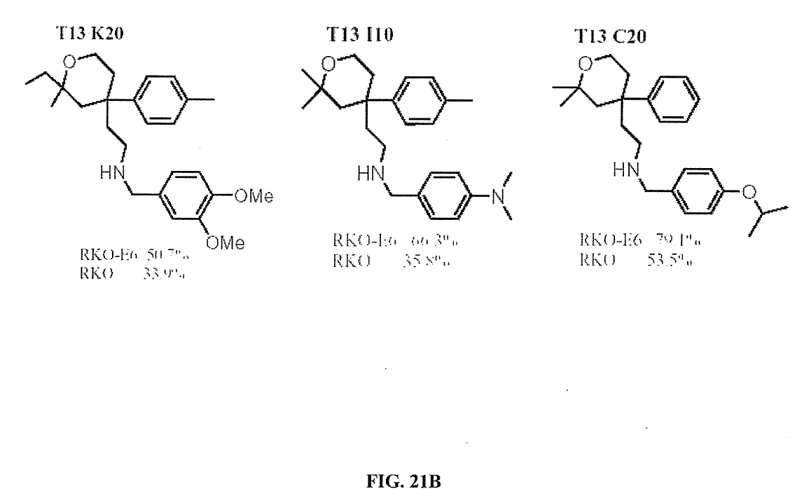

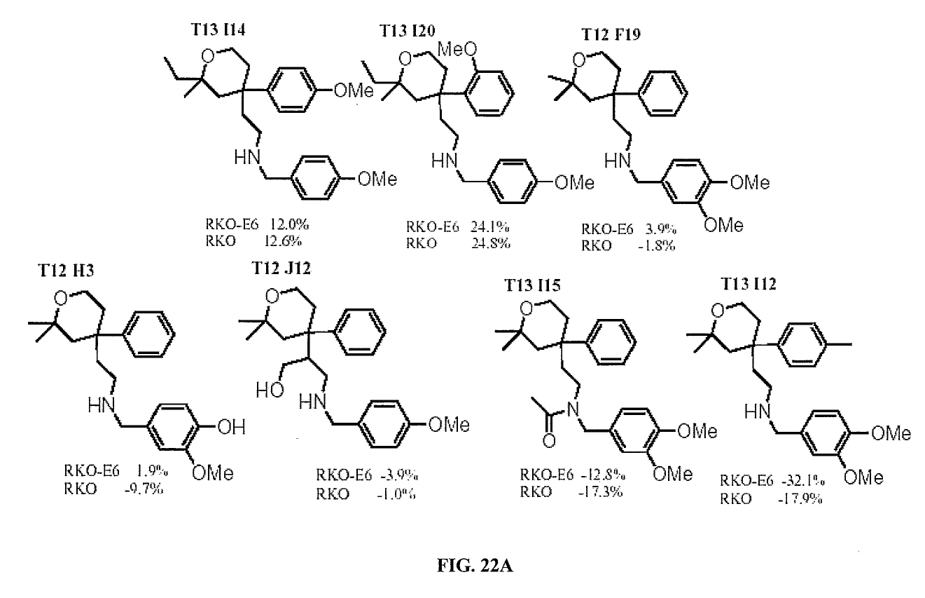

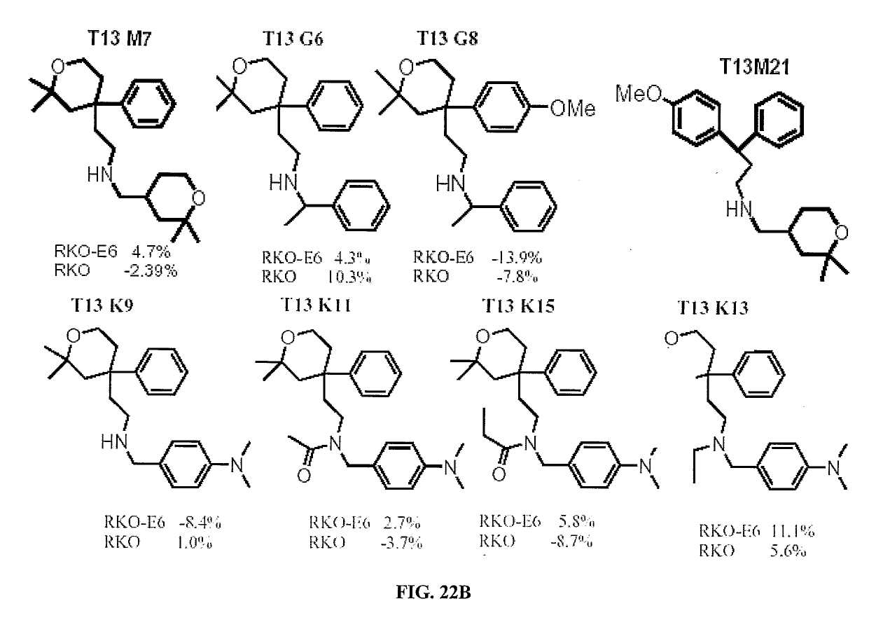

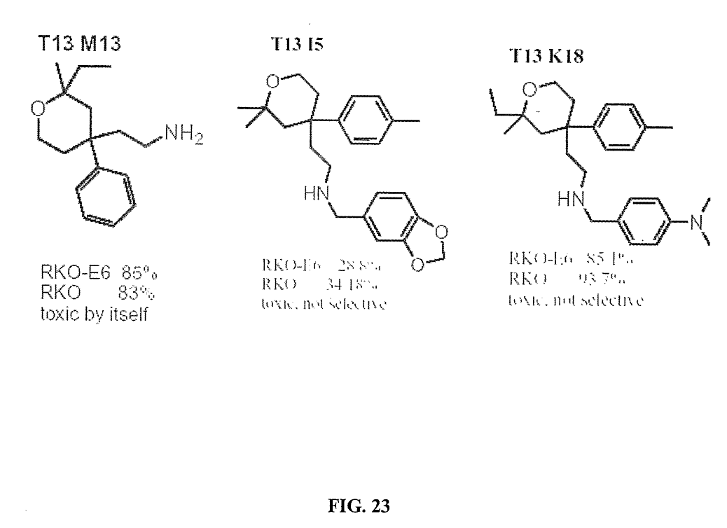

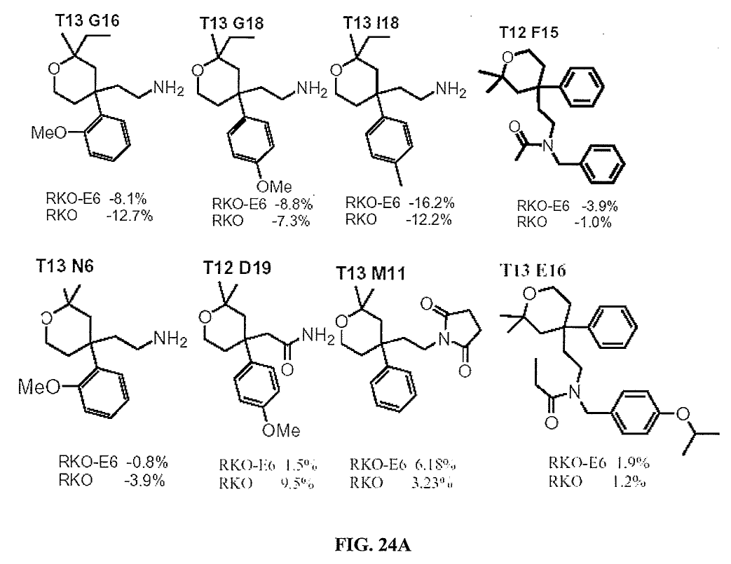

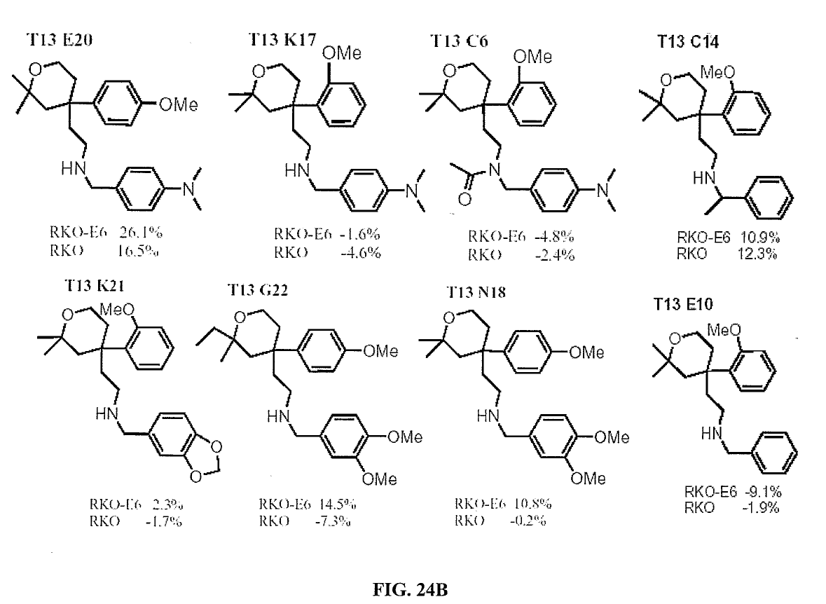

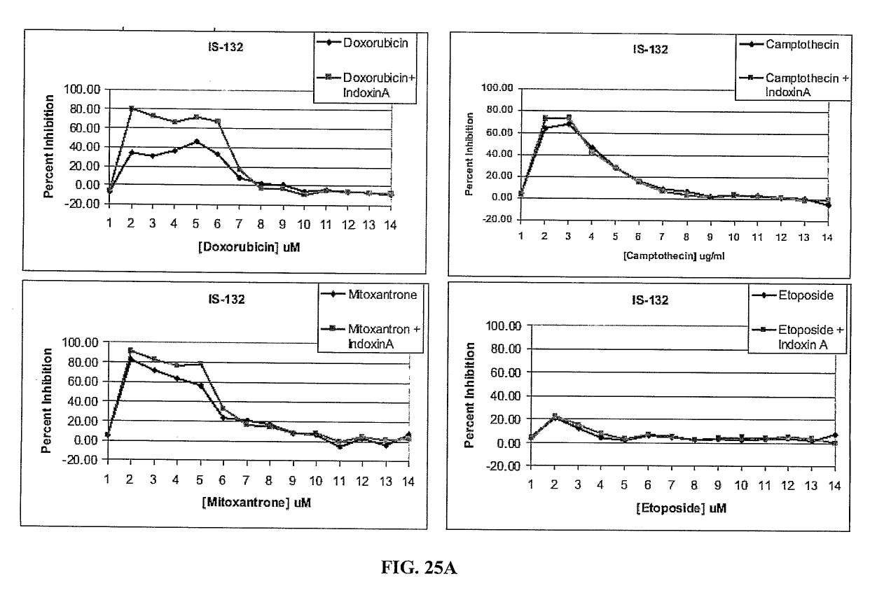

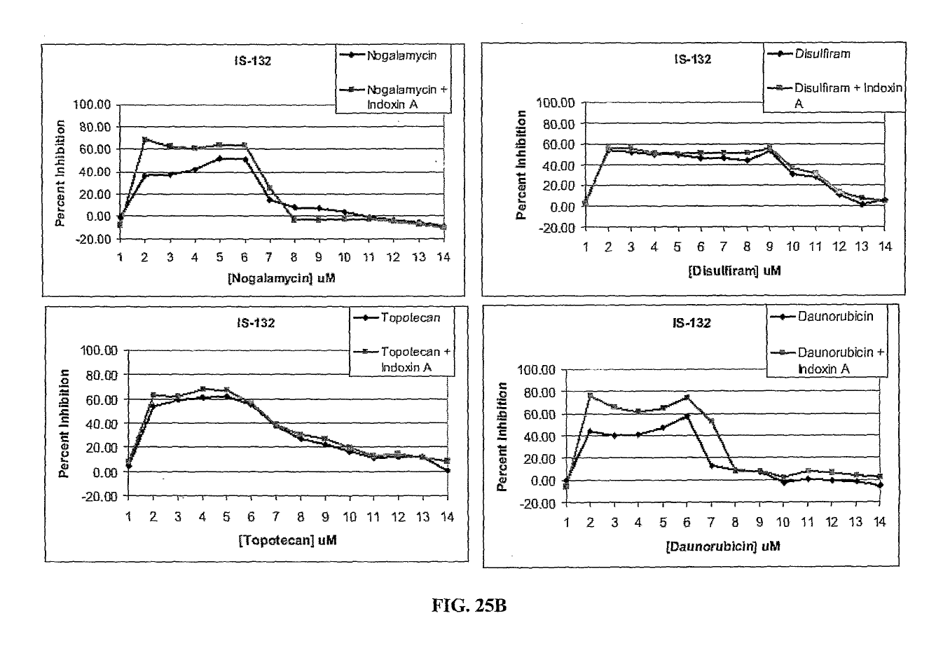

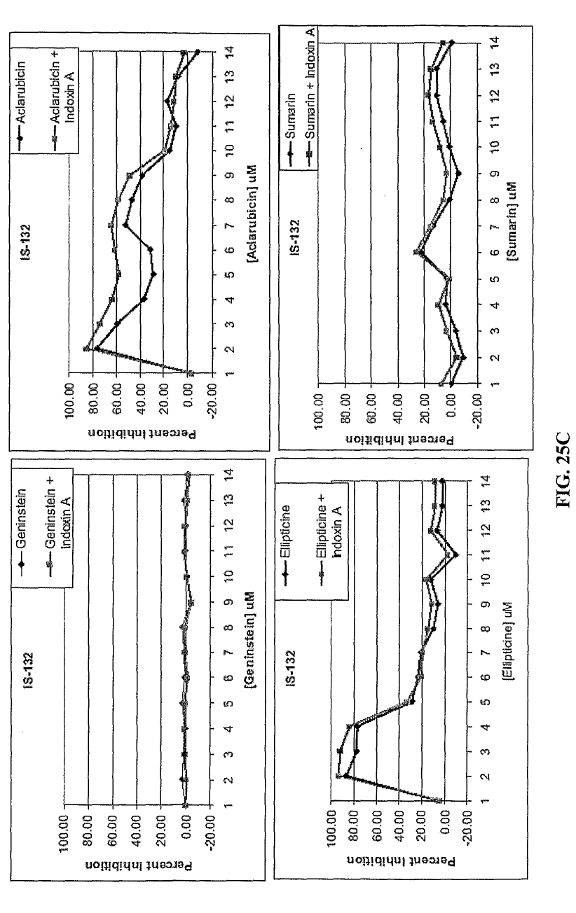

To identify such compounds, a secondary screen was performed in which 272 of the primary hit compounds were tested in two-fold dilution series in RKO cells in the absence of doxorubicin and in RKO-E6 cells in the presence of doxorubicin (the protocol for the screen, the tested compounds and all activity data are provided in the supplementary materials). This screen allowed for the elimination of compounds that are equally lethal to RKO cells in the absence of doxorubicin; such a filter should eliminate compounds that induce cell death via pathways independent of E6 and doxorubicin. 88 compounds were selected for further analysis that exhibited at least 20% greater cell growth inhibition in RKO-E6 cells in the presence of doxorubicin compared to the lethality of these compounds alone in RKO cells (see FIGS. 21-25 for additional compounds examined during the combinatorial screening process and their respective cell growth inhibition activities).

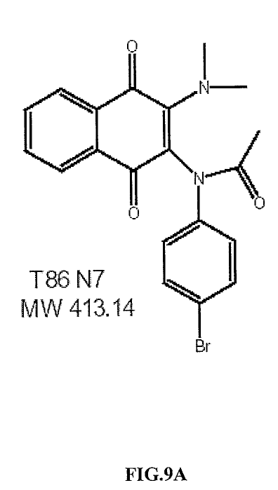

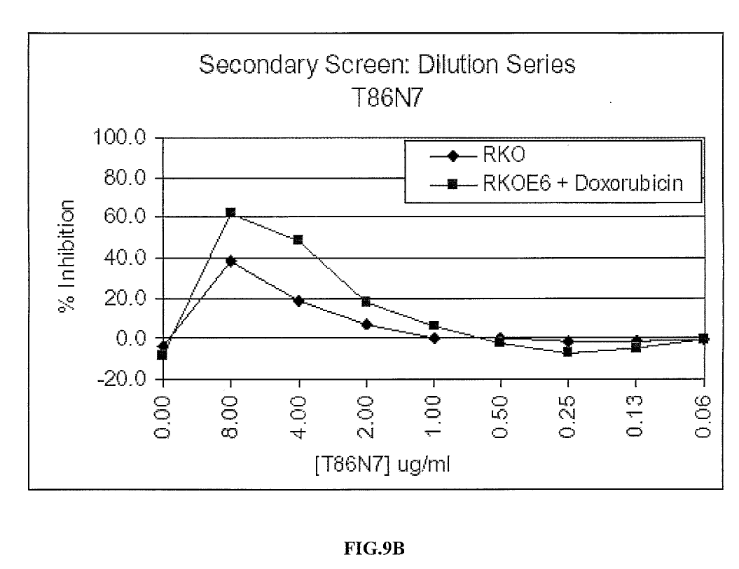

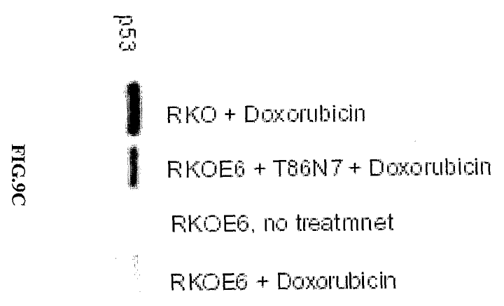

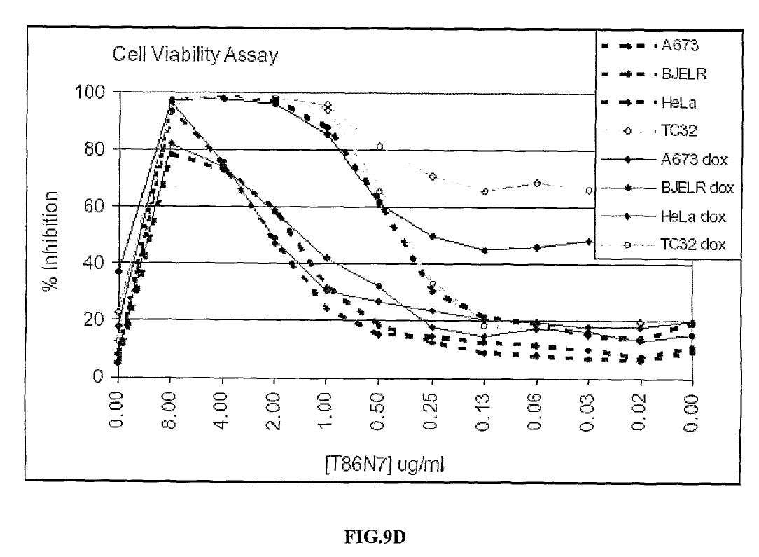

Compounds that overcome E6-induced doxorubicin resistance might act by upregulating p53. Thus, all 88 compounds were tested for their ability to upregulate p53 in RKO-E6 cells using western blotting. Only one of the 88 compounds (designated T86N7) triggered a moderate ˜50% upregulation of p53 (the structure and activity of this compound are provided in the supplementary materials), indicating that most compounds restored doxorubicin lethality without directly inhibiting E6AP or E6 or even upregulating p53 by other means (FIG. 9). However, given that all 88 of these compounds increased the sensitivity of RKO-E6 cells to doxorubicin (modestly or profoundly), the basis for their desired re-sensitization activity was investigated.

To further group the 88 compounds based on their mechanism of action, a pathway-based analysis was developed using a co-treatment strategy. For these assays, the microtubule inhibitor podophyllotoxin, the topoisomerase-I-based DNA-damaging agent camptothecin, and doxorubicin itself were selected. The anti-mitotic drug podophyllotoxin inhibits tubulin polymerization, leading to apoptosis, while camptothecin and doxorubicin recruit topoisomerase I and topoisomerases II, respectively, to introduce DNA strand breaks, which in return induce apoptosis.

RKO-E6 cells were co-treated with selected compounds and with podophyllotoxin, camptothecin, or doxorubicin. Compounds that increased sensitivity to all three of these lethal compounds were assumed to act through general cell death mechanisms and were eliminated from further consideration. Compounds that enhanced sensitivity to both camptothecin and doxorubicin, but not podophyllotoxin, presumably operate downstream of the DNA-damage response. Finally, compounds that synergized only with doxorubicin were likely to operate at the level of, or upstream of, topoisomerase II proteins.

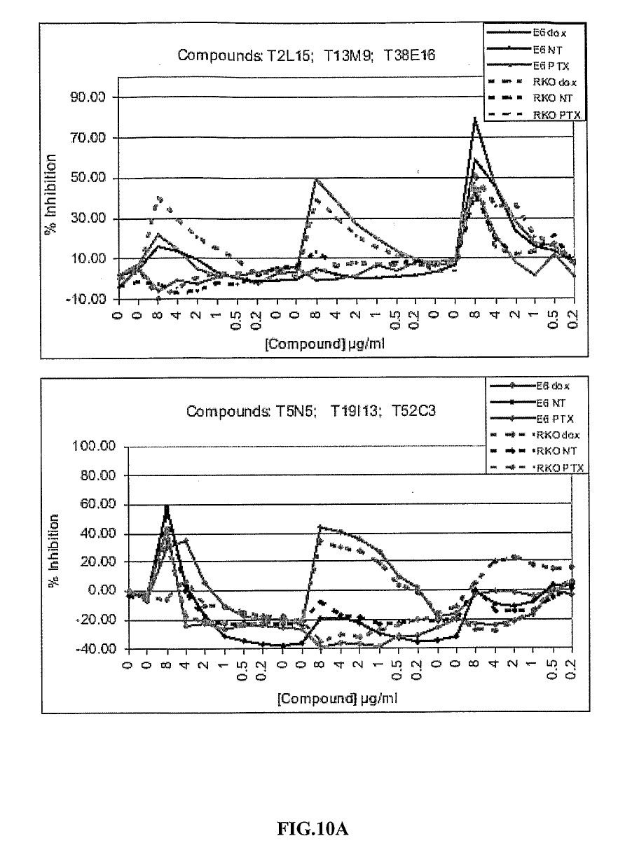

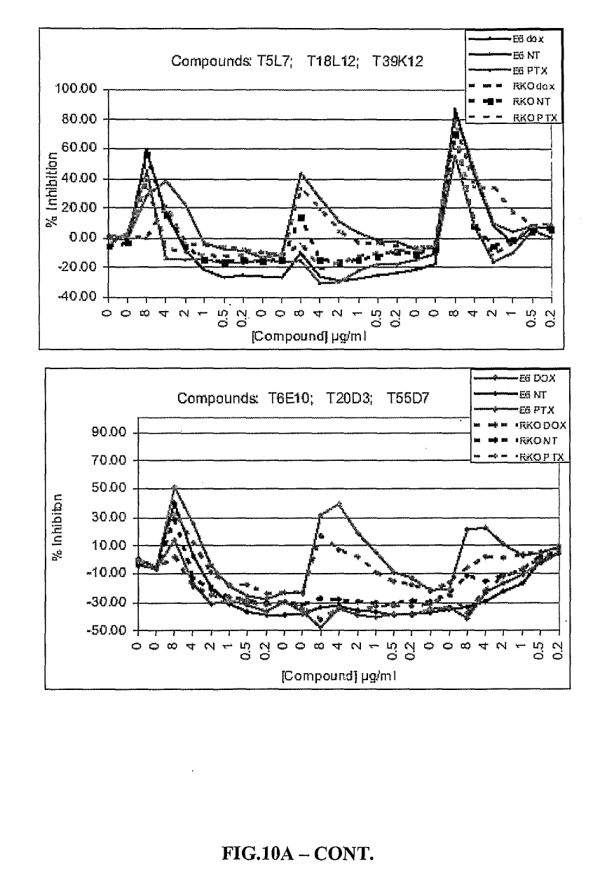

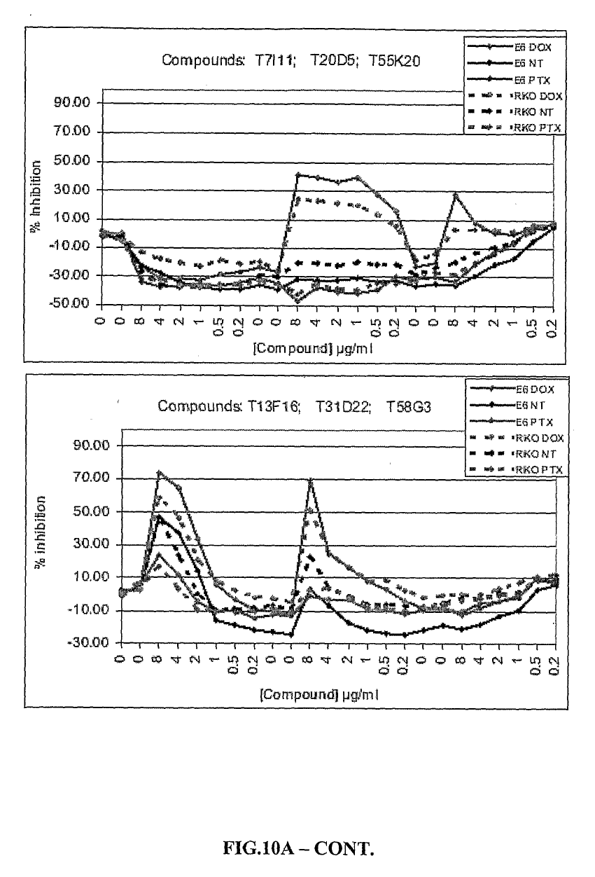

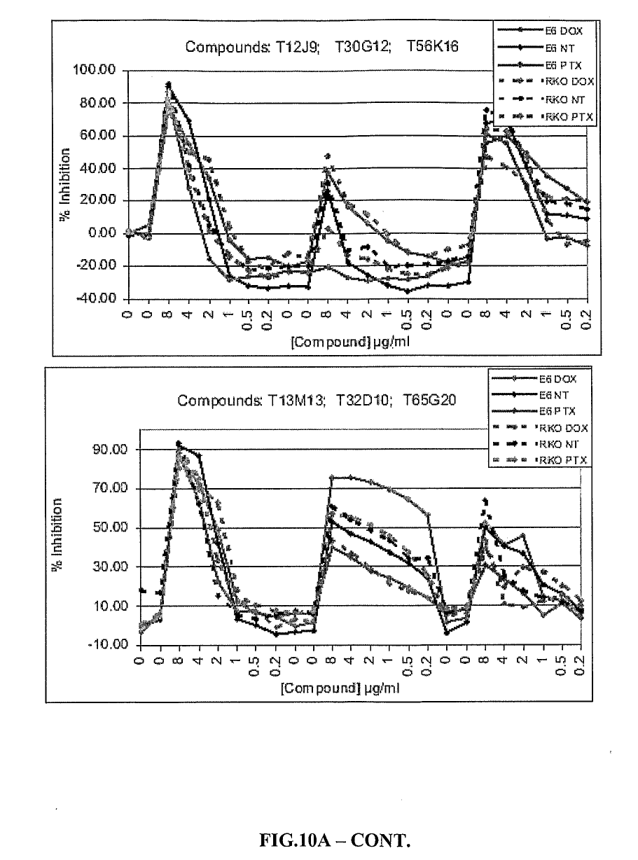

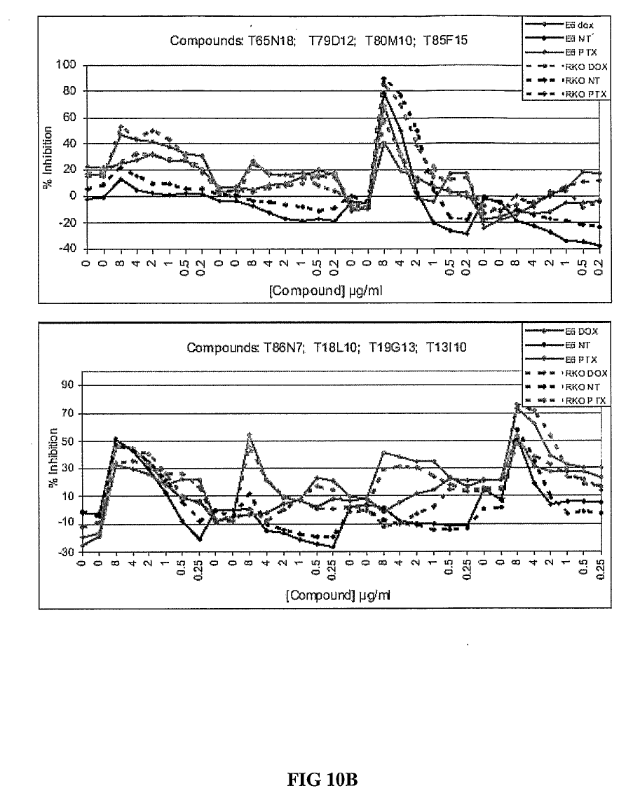

For this tertiary screen, the 24 most selective and active compounds from the secondary screen were selected. Representative compounds from each structural class were tested. Each of these compounds was tested in four replicates in RKO and RKO-E6 cell lines alone, and with doxorubicin or podophyllotoxin (these compounds and activity data are provided in the supplementary materials; FIGS. 10A-B). Compounds that potentiated podophyllotoxin's toxicity were eliminated from further consideration, while compounds that confirmed their ability to selectivity increase doxorubicin's lethality in RKO-E6 and/or RKO cells were analyzed in more detail.

Below, structurally and functionally related groups of compounds that emerged from these screens and candidate mechanisms of action are described.

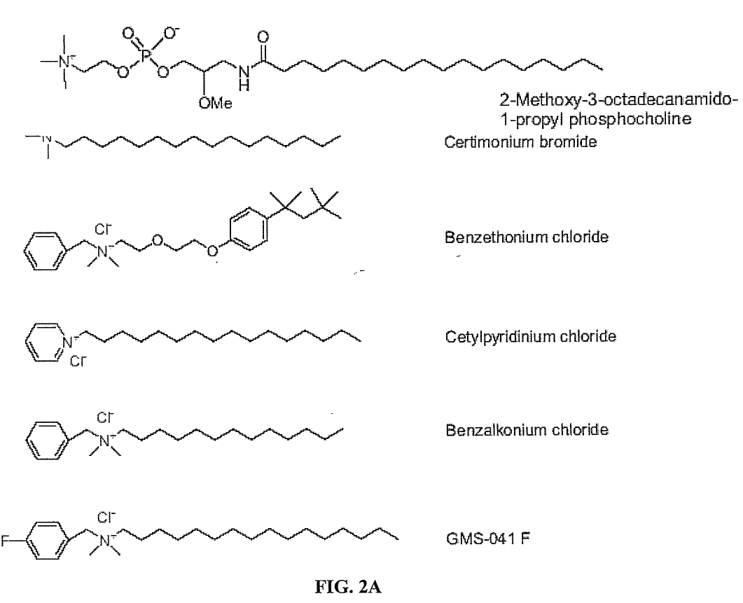

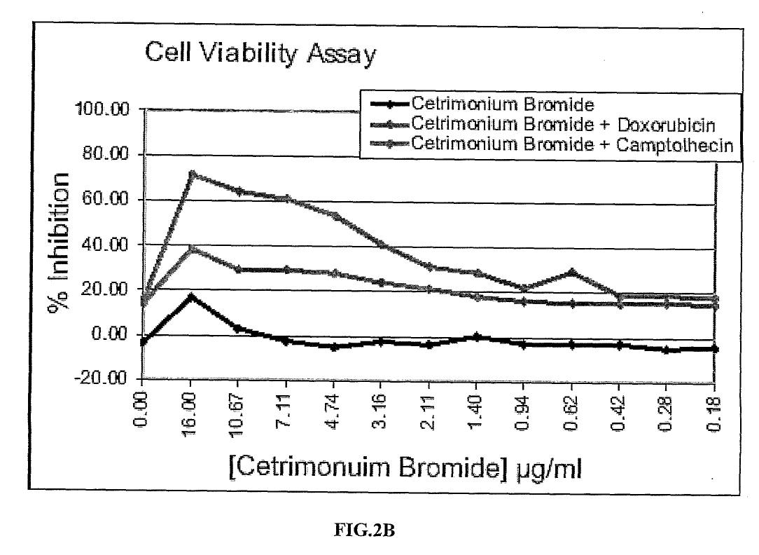

Quaternary ammonium compounds: The first class of doxorubicin-enhancing agents comprised quaternary ammonium compounds (QACs), such as cetrimonium bromide, cetylpyridinium chloride, benzethonium chloride, and benzalkonium chloride. These compounds selectively increased doxorubicin's lethality in RKO-E6 cells, but did not synergize with camptothecin (FIG. 2). QACs have been reported to act as membrane-disrupting agents by solubilizing the cytoplasmic membrane in bacteria and yeast. The ability of such compounds to disrupt the cytoplasmic membrane of tumor cells and increase their permeability could facilitate doxorubicin's uptake and lethality in RKO-E6 cells.

Benzalkonium salts have effects on a variety of cells (including T cells), downregulate tumor necrosis factor expression, and are effective bactericidal, fungicidal, and virucidal agents with pleiotropic (direct and immunologically-mediated) inhibitory activity against pathogens (Patarca and Fletcher, 1995; Patarca et al., 2000). Although these compounds have been primarily used as anti-infective agents, an analog of benzalkonium chloride has been reported to inhibit proliferation of several human cancer cell lines (Gastaud et al., 1998). These authors proposed that the ability of quaternary ammonium functional groups to act as alkylating agents might be responsible for their inhibitory effect on tumor cells. However, benzethonium chloride and benzalkonium chloride did not induce apoptosis in RKO-E6 cells in the absence of doxorubicin, casting doubt on this explanation. In addition, these compounds did not induce cell cycle arrest, which is typically caused by DNA alkylating agents, and did not upregulate p53, p21, or caspase 3, implying a different mechanism of activity. These compounds may be fairly benign agents that could be used for overcoming resistance to doxorubicin.

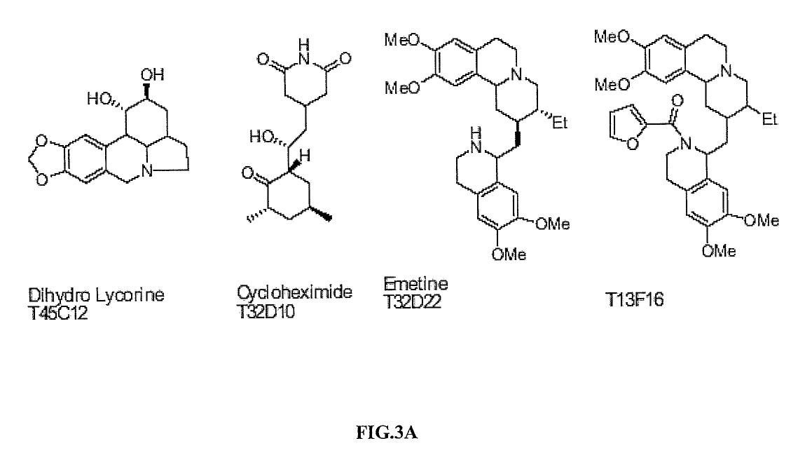

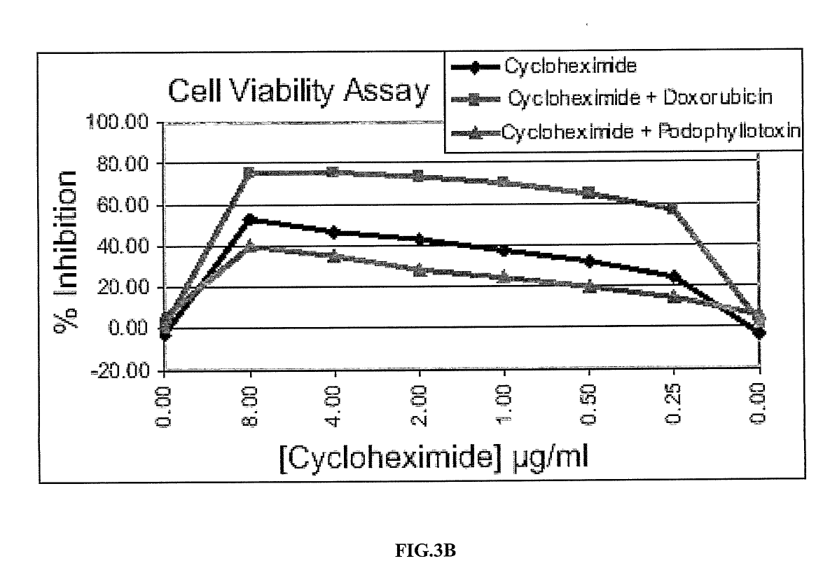

Protein synthesis inhibitors: Cycloheximide, emetine, and dihydrolycorine potentiated doxorubicin's lethality in RKO-E6 cells, although at higher concentrations they exhibited lethality on their own (FIG. 3). Protein synthesis inhibitors have been reported to sensitize cells to members of the Tumor Necrosis Factor (TNF) family (Choi et al., 2004) and to cisplatin (Budihardjo et al., 2000). Contrary to these observations, however, there are several reports that cycloheximide antagonizes doxorubicin-induced apoptosis (Bonner and Lawrence, 1989; Furusawa et al., 1995). It is likely that protein synthesis inhibition has diverse effects, depending on the concentrations used for treatment and the genetic makeup of the target cells.

Consistent with their reported inhibitory effects on protein synthesis, cycloheximide caused a disappearance of p53 and p21 in RKO-E6 cells, even when co-treated with doxorubicin. On the other hand, cycloheximide caused increased topoisomerase IIα levels in RKO-E6 cells.

T13F16 is an analog of emetine with a modest activity profile in our viability studies (FIG. 3), however, it appears to lack potent protein synthesis inhibitor activity and did not prevent up-regulation of p53 and p21 in response to doxorubicin's treatment. This analog may have fewer undesired side effects than the more potent protein synthesis inhibitors. In any case, protein synthesis inhibitors appear, unexpectedly, to constitute an effective class of agents for restoring doxorubicin lethality.

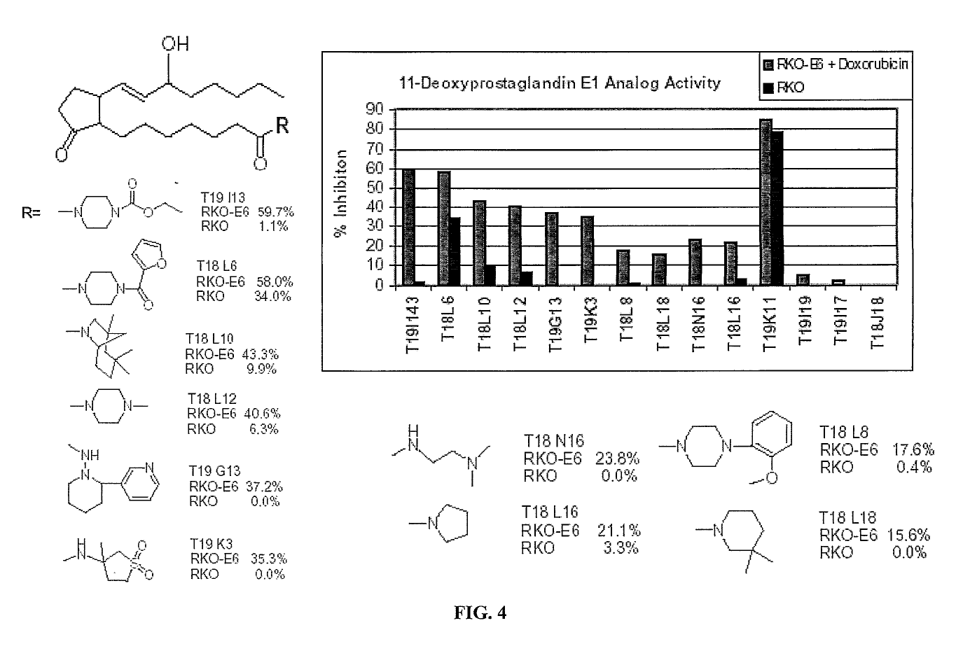

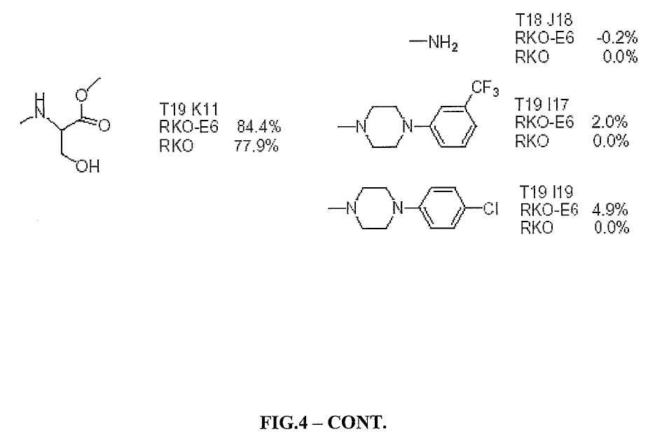

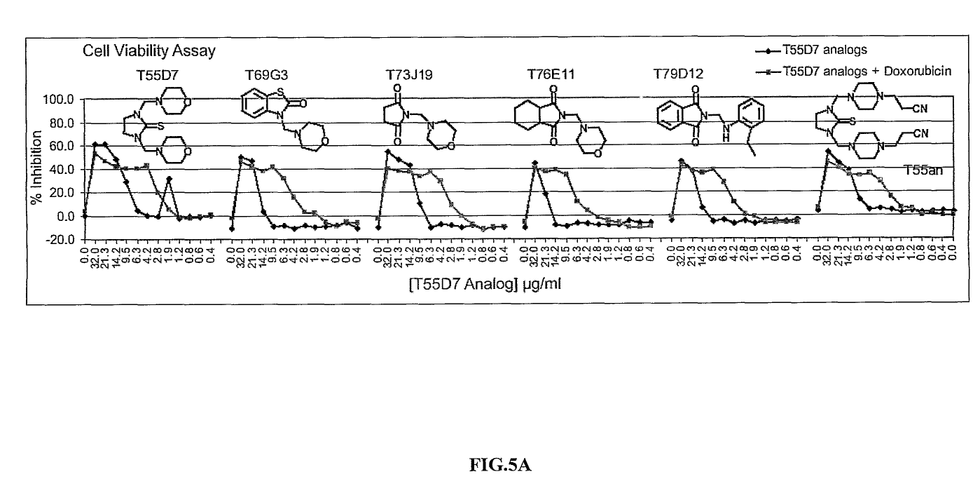

11-Deoxyprostglandin E1: 11-deoxyprostglandin E1 analogs (e.g. T19I13) were isolated in the screen. Depending upon the amide functionality, these compounds exhibited a broad range of activity and selectivity, while the parent compound 11-deoxyprostaglandin E1 showed no activity in this screen (FIG. 4). T19I13 induced late S/G2-phase arrest in RKO-E6 cells, while no changes in cell cycle was observed in RKO cells. Upon co-treatment with doxorubicin and T19I13, RKO-E6 cells accumulated in S/G2 phase, while RKO cells populated G1 and late S/G2 phase. T19I13 selectively increased doxorubicin's lethality, while not synergizing with podophyllotoxin or camptothecin. However, a rapid loss activity of T19I13 in cell culture was observed, impeding further testing of this analog series, likely due to loss of the allylic hydroxyl.

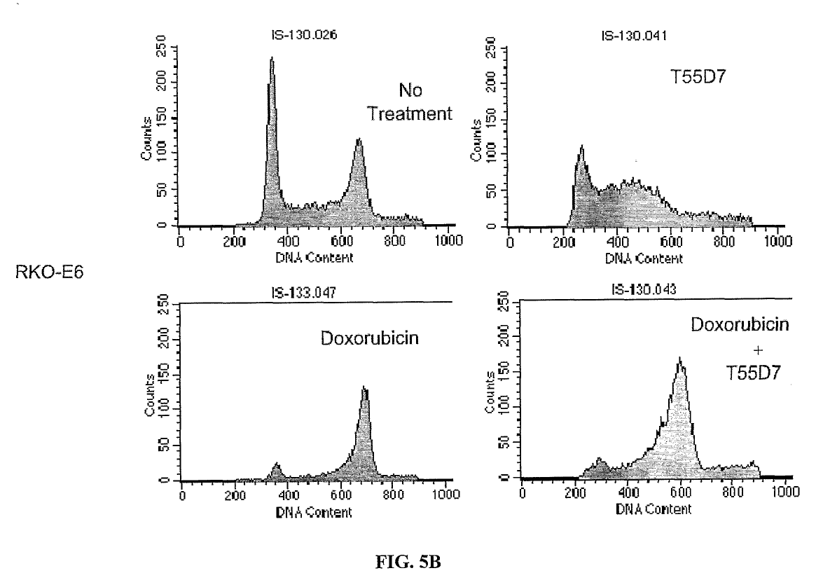

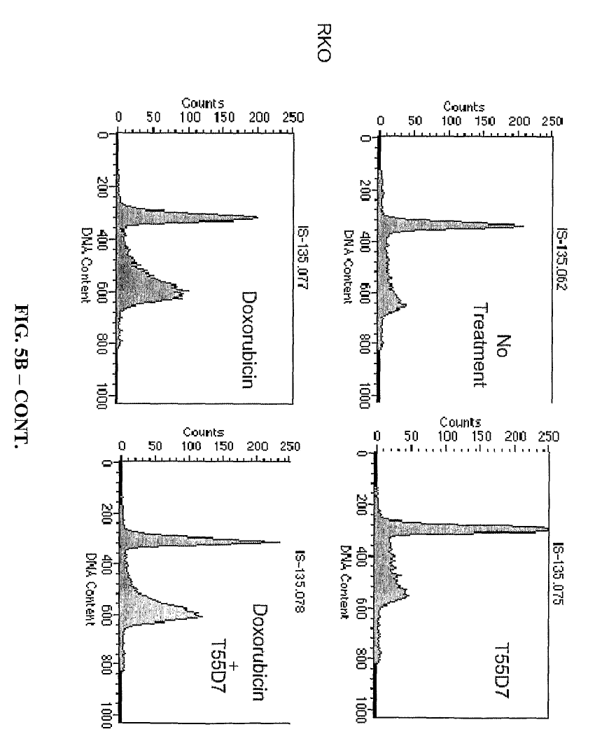

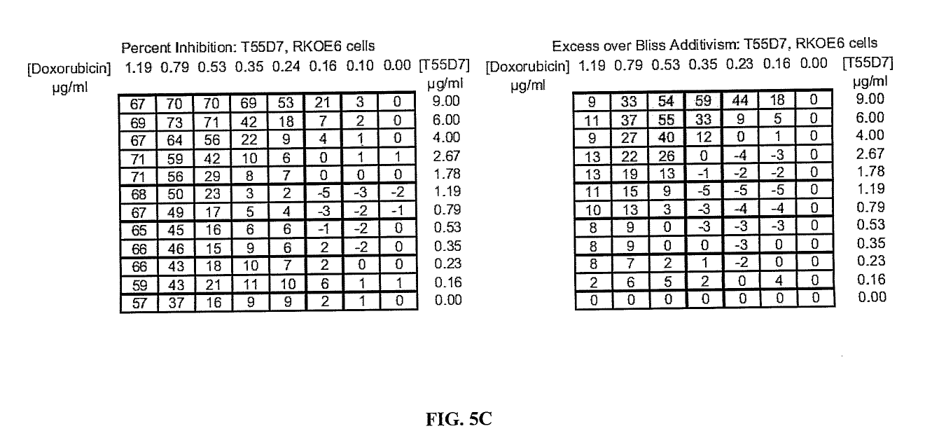

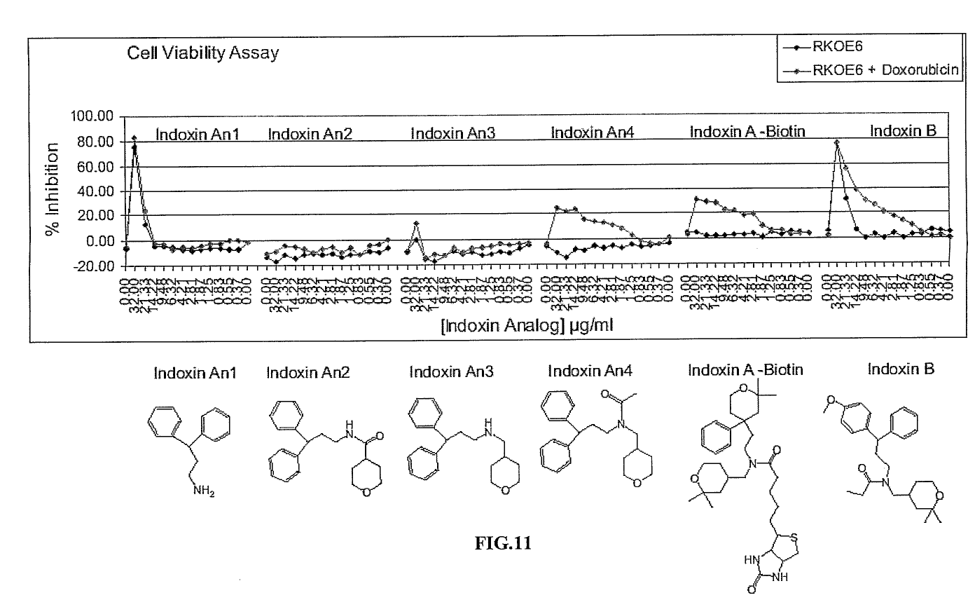

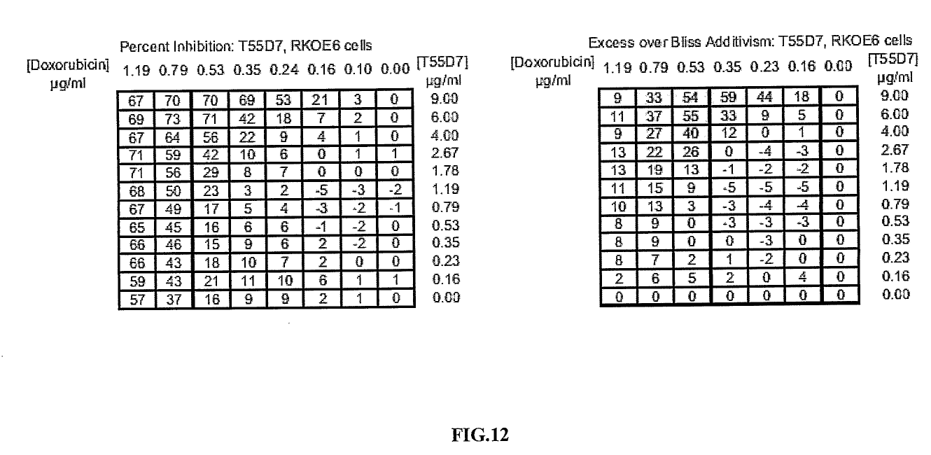

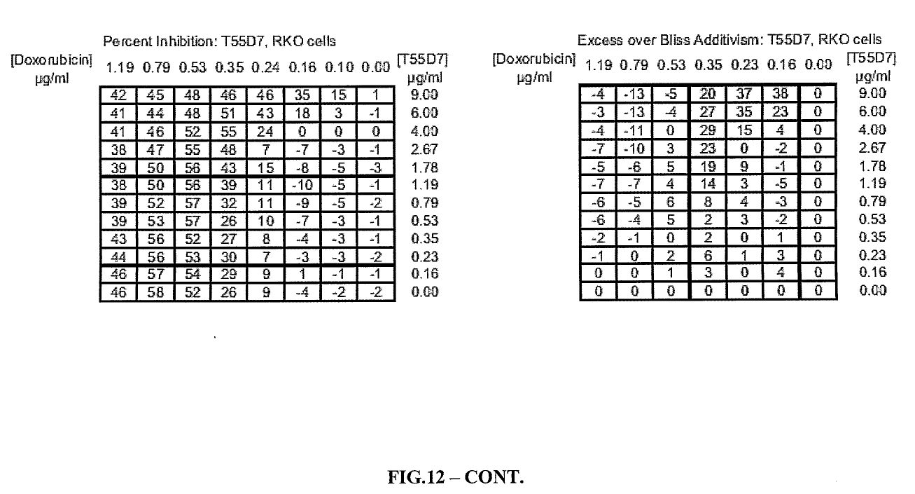

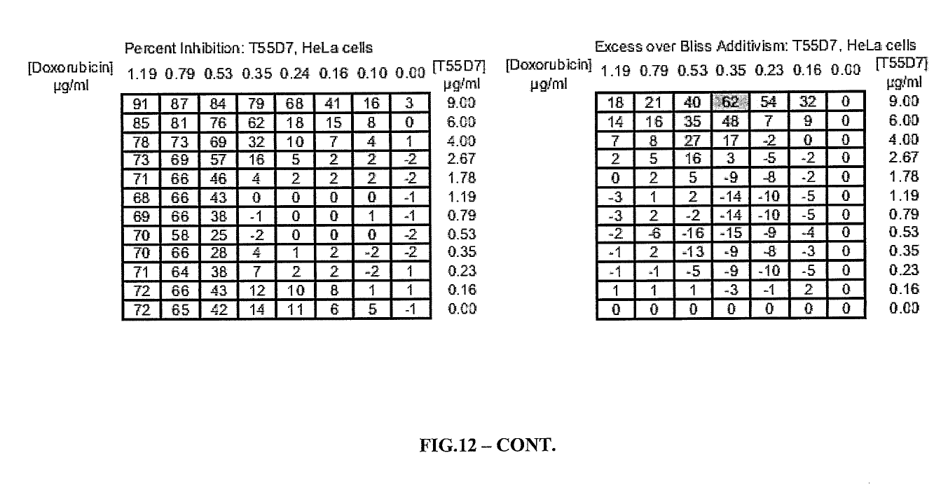

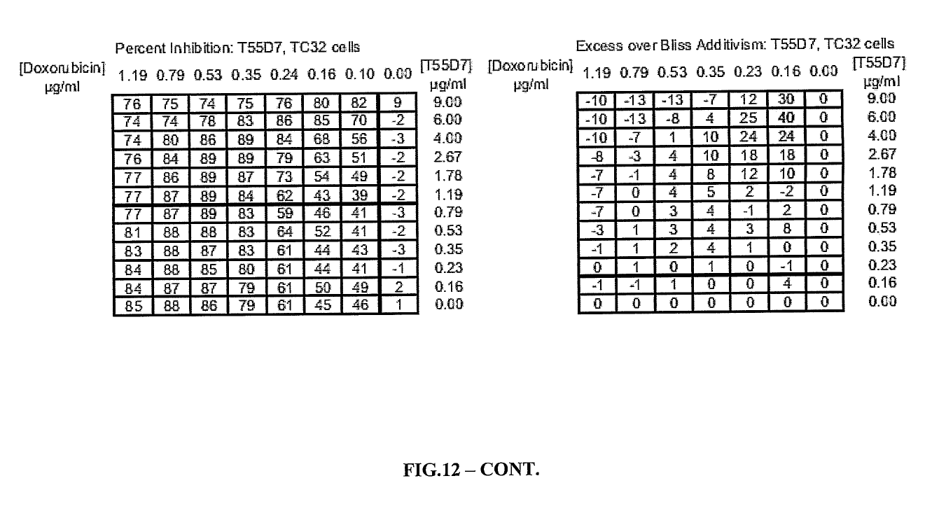

1,3-bis(4-morpholinylmethyl)-2-imidazolidinethione (T55D7) and related analogs: 1,3-bis(4-morpholinylmethyl)-2-imidazolidinethione (T55D7) and its analogs represent a class of small molecules. Low micromolar concentrations of these compounds were found to increase both doxorubicin's and camptothecin's lethality in RKO and RKO-E6 cells (FIG. 5A). T55D7 was studied in more detail as a prototypical member of this compound class; it upregulated doxorubicin's potency in other cell lines: HeLa, TC32 and A673. However, T55D7 did not increase the microtubule inhibitor podophylotoxin's lethality, indicating a DNA-damage related mechanism of action. Only at very high concentrations (30-100 micromolar) did T55D7 and its analogs exhibit doxorubicin-independent lethality (FIG. 5A). To investigate whether T55D7 truly synergizes with doxorubicin, RKO, RKO-E6, HeLa, and TC32 cell lines were treated with a combination dose matrix of T55D7 and doxorubicin (FIG. 5C). The Bliss Independence model predicts the combined response C for two single compounds with effects A and B according to the relationship C=A+B−A*B and represents one of the most stringent requirements for synergy (Keith et al., 2005). According to this model, the excess over predicted Bliss independence represents the synergistic effect of the combination treatment at a given pair of concentrations. Based on this analysis, T55D7 exceeded Bliss Independence with doxorubicin at multiple doses; the most significant potentiation of doxorubicin's lethality was found in RKO-E6 cells (FIG. 5C and FIG. 12).

Western blot analysis of RKO-E6 cells co-treated with doxorubicin and T55D7 analogs showed no changes in p53, p21, or MDM2 levels, compared to doxorubicin-treated cells. T55D7 and its analogs also did not change p53 concentration in the absence of doxorubicin, implying that the ability of these compounds to potentiate doxorubicin's toxicity is not caused by their ability to induce DNA damage or affect processes upstream of 53.

T55D7 treatment resulted in pronounced S-phase arrest in RKO-E6 cells, while virtually no changes in the cell cycle were observed in RKO cells (FIG. 5B). When RKO and RKO-E6 cells were co-treated with T55D7 and doxorubicin, a broadening of the G2 phase and an increase in S phase in RKO-E6 cells were observed, while RKO cell-cycle distribution was similar to doxorubicin-only treatment. Thus, T55D7 and related analogs have the ability to enhance the lethality of DNA-damaging agents in numerous tumor cell lines. These compounds might be developed into adjuvant therapies for doxorubicin and other DNA-damaging agents.

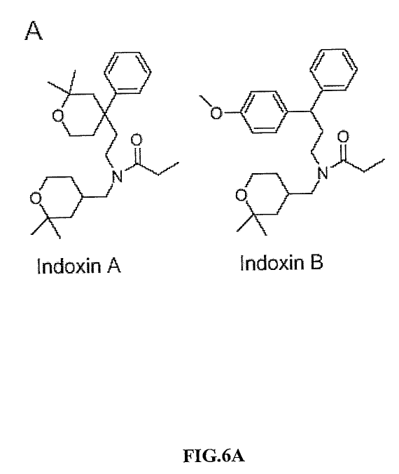

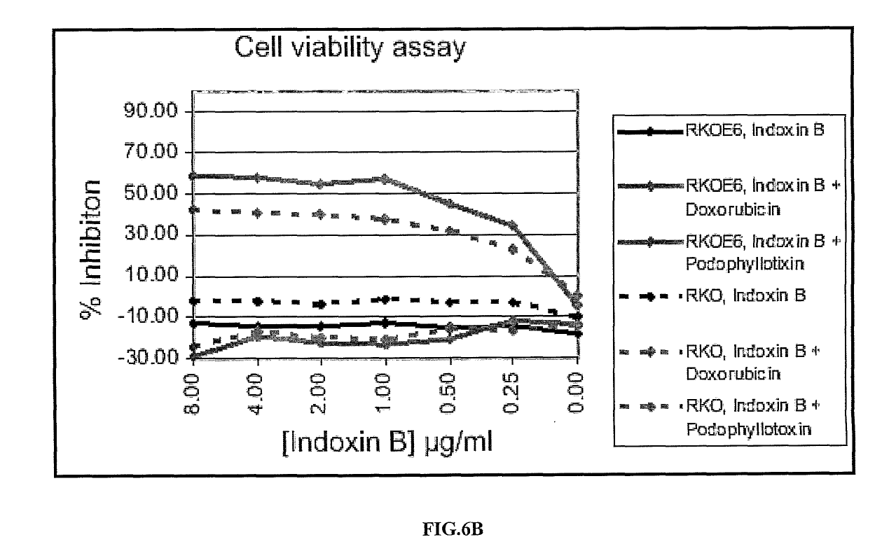

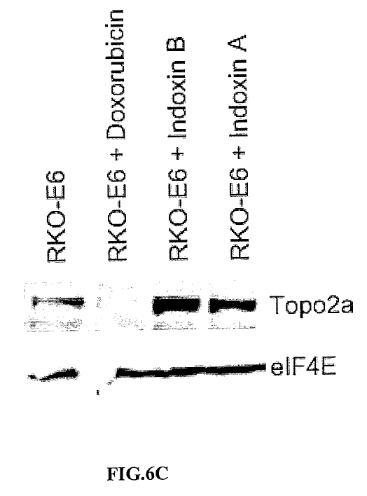

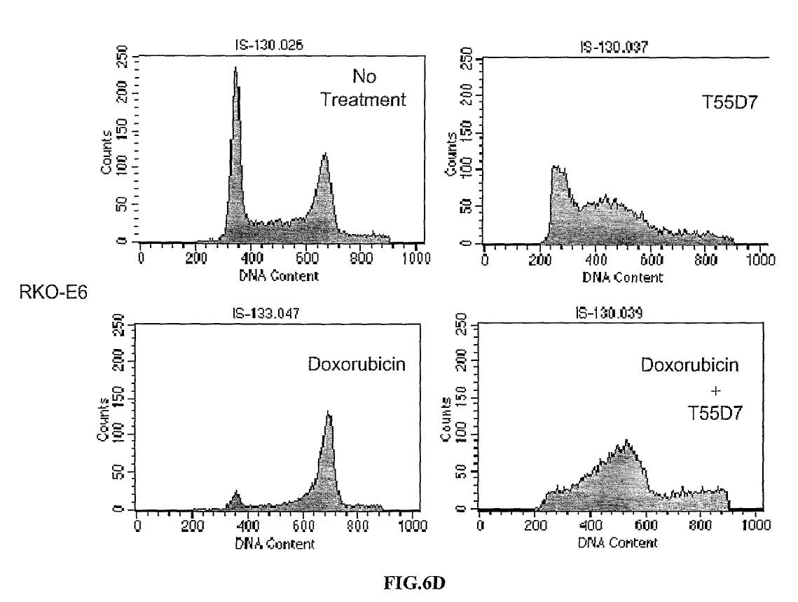

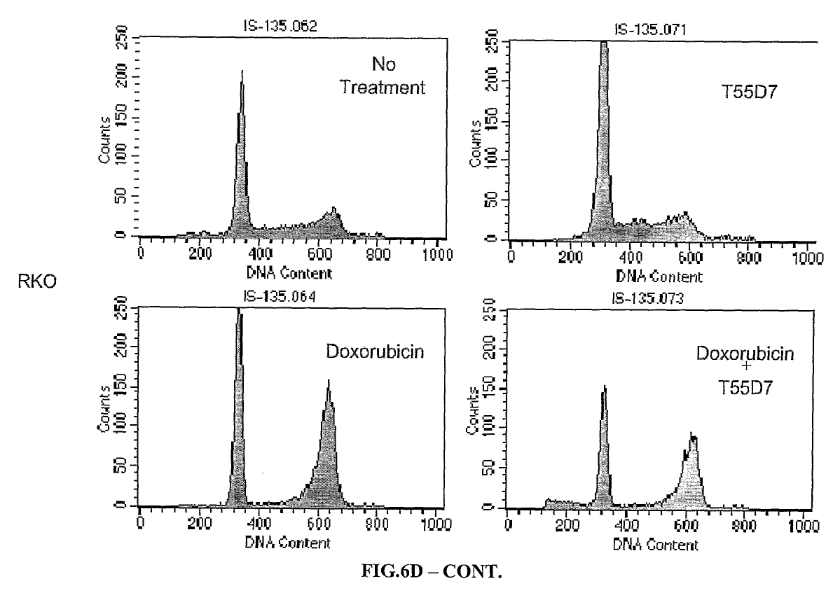

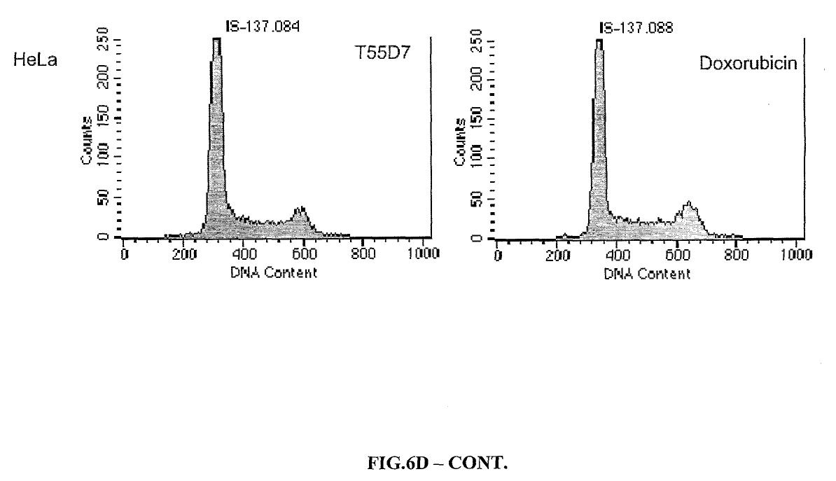

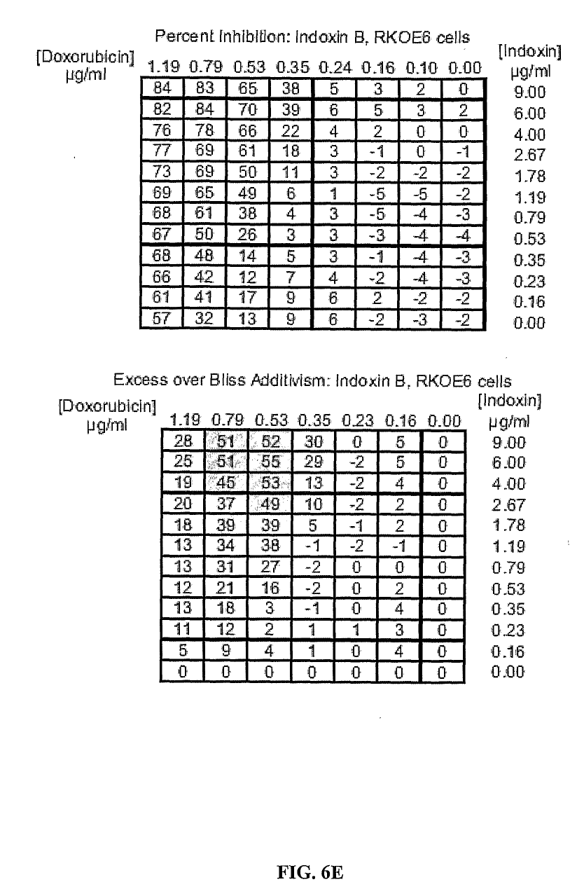

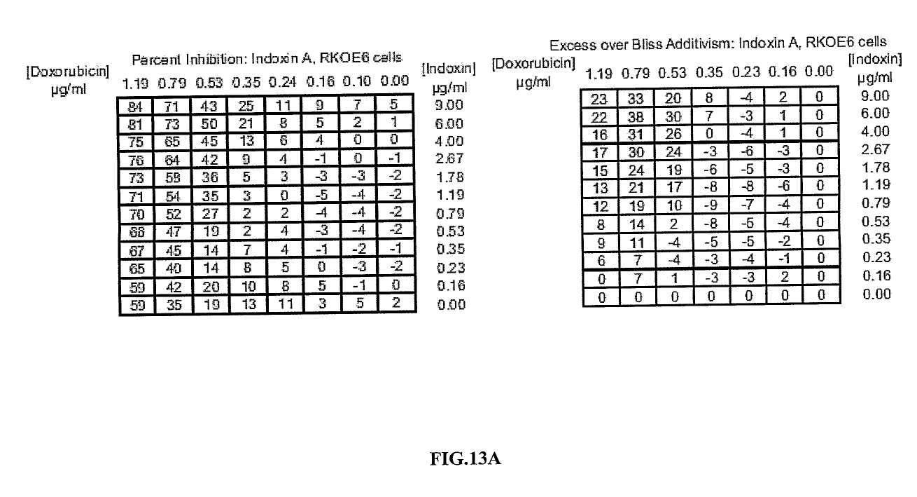

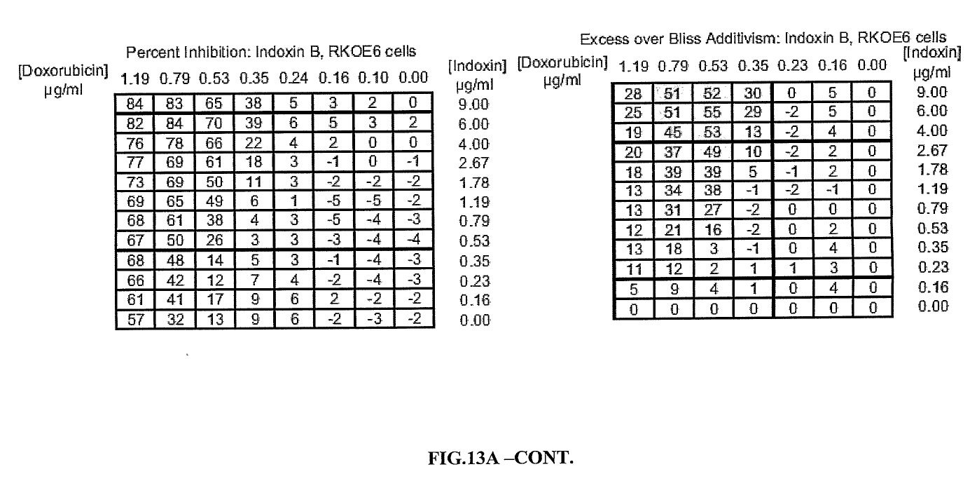

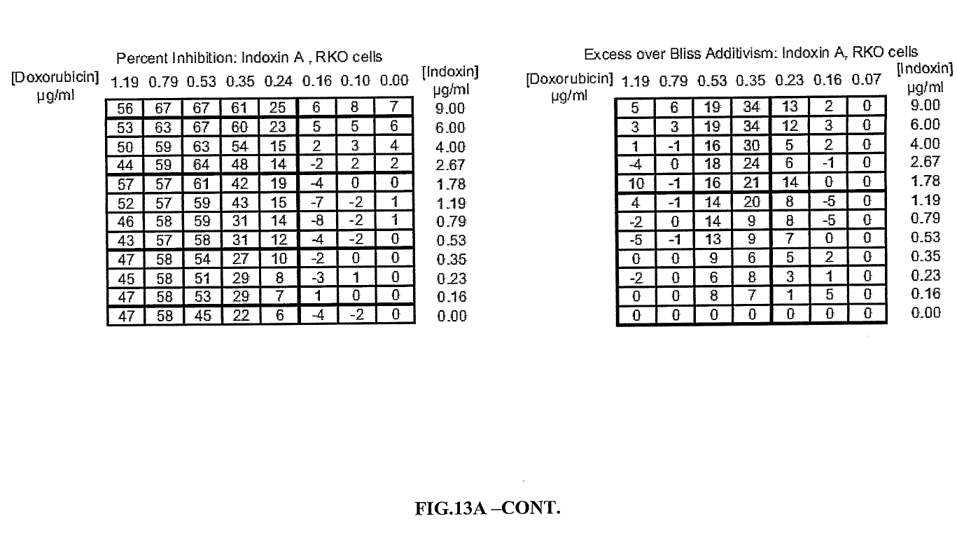

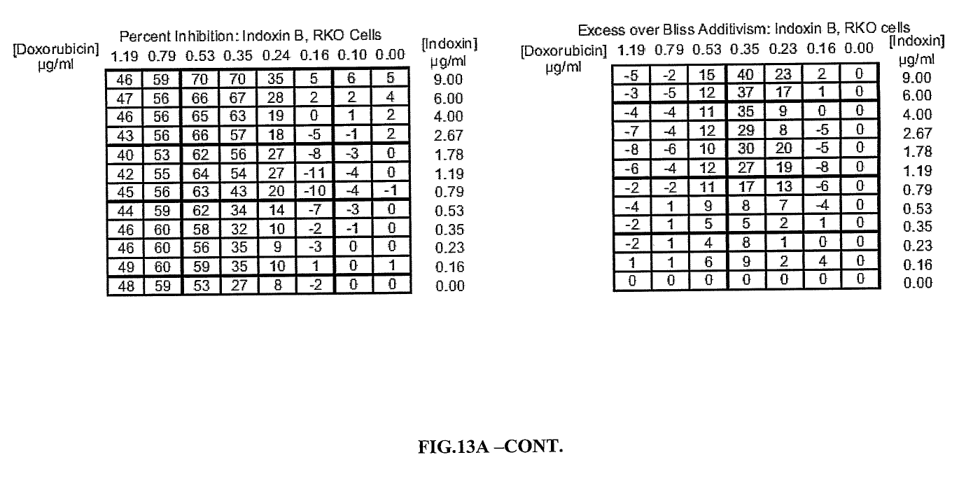

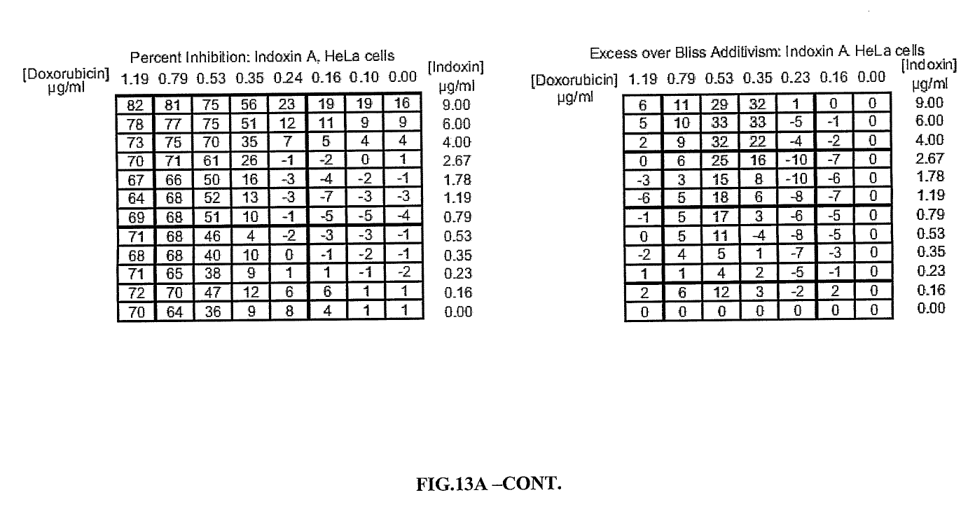

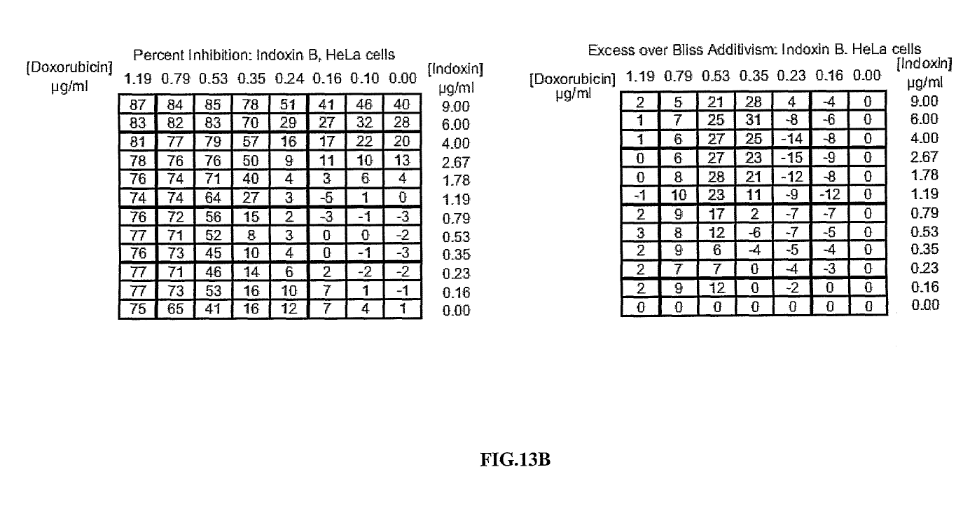

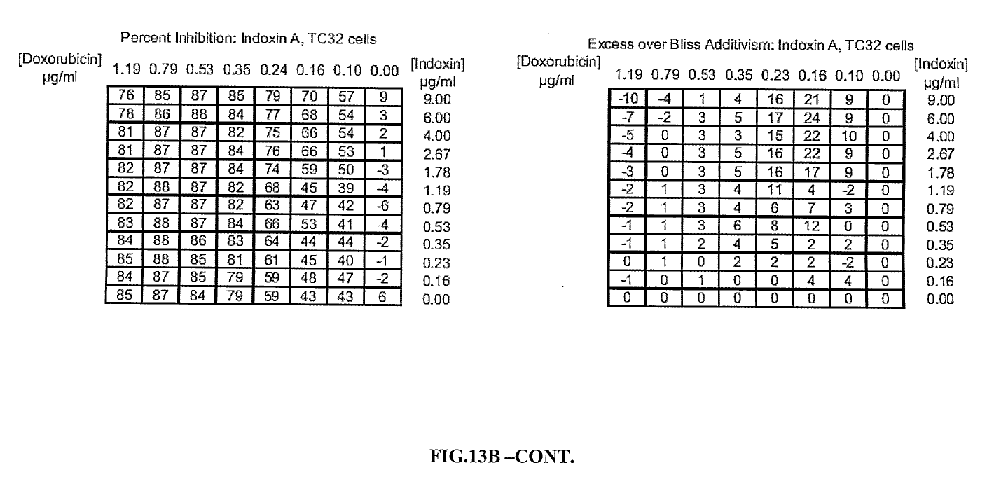

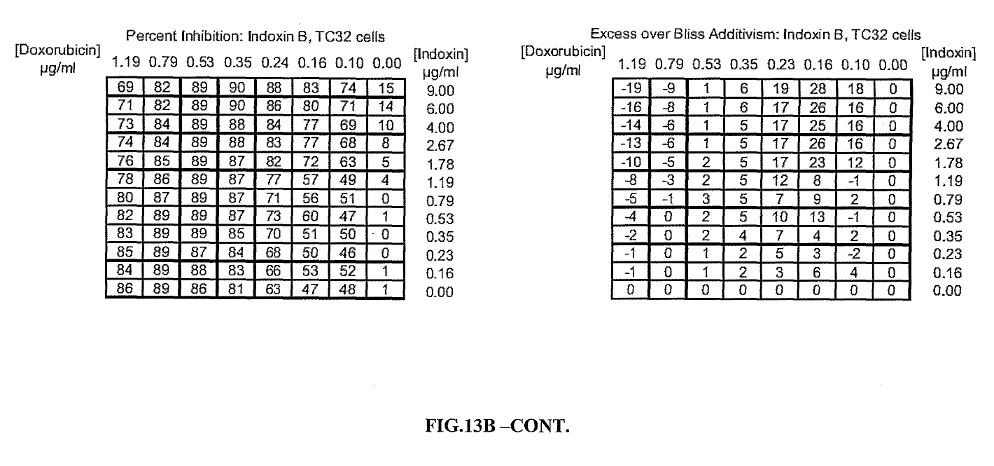

Indoxins: T13M9 and T20D5 are representative compounds referred to herein as indoxins for to their ability to Increase Doxorubicin's lethality selectively. These small molecules selectively increased doxorubicin's lethality in RKO, RKO-E6, HeLa and in TC32 cells, while showing no synergy with camptothecin or podophyllotoxin (FIG. 6B). A Bliss Independence analysis showed that indoxins and doxorubicin have more than additive effects in all four cell lines (FIG. 6E and FIGS. 13A-B). This doxorubicin-selectivity implied a topoisomerase II-related mechanism of action. Indeed, direct upregulation of topoisomerase IIα was detected in RKO and RKO-E6 cells treated with indoxin A and indoxin B using western blotting (FIG. 6C). Interestingly, indoxins also induced S-phase arrest in RKO-E6 cells, while not affecting RKO or HeLa cell-cycle distribution (FIG. 6D). Topoisomerase IIα is upregulated during S-phase and G2/M phase. However, since increased topoisomerase IIα levels were observed in RKO and RKO-E6 cells, but S-phase arrest was induced only in RKO-E6 cells, these two events seems to be independent in this assay system.

A series of indoxin analogs were synthesized to assess functionalities necessary for their activity. Structure-activity analysis of indoxin analogs revealed that an acyl group must be present on the secondary amine for activity to be observed. However, when the acetyl or propanoyl functionality was substituted with a biotin-linked acyl group, indoxin A retained some activity and selectivity, indicating that larger substituents can be introduced at this site (FIG. 6 and FIG. 11).

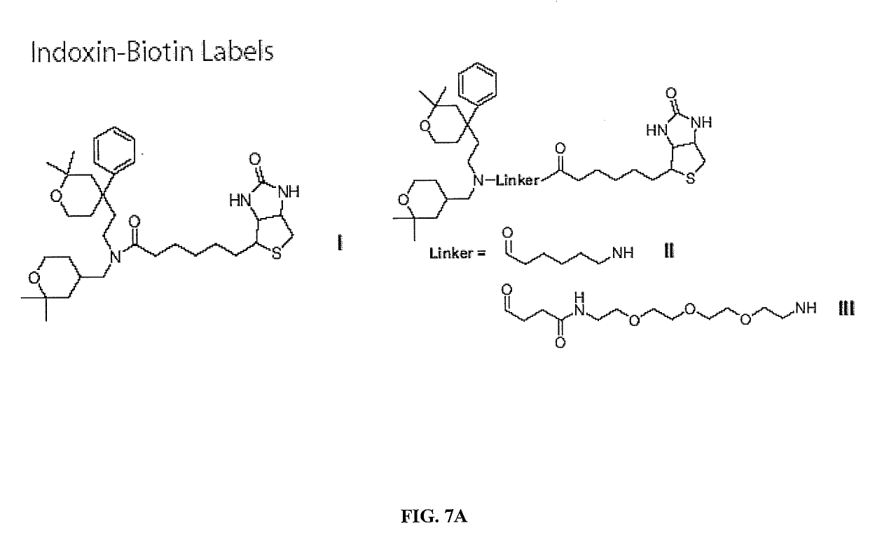

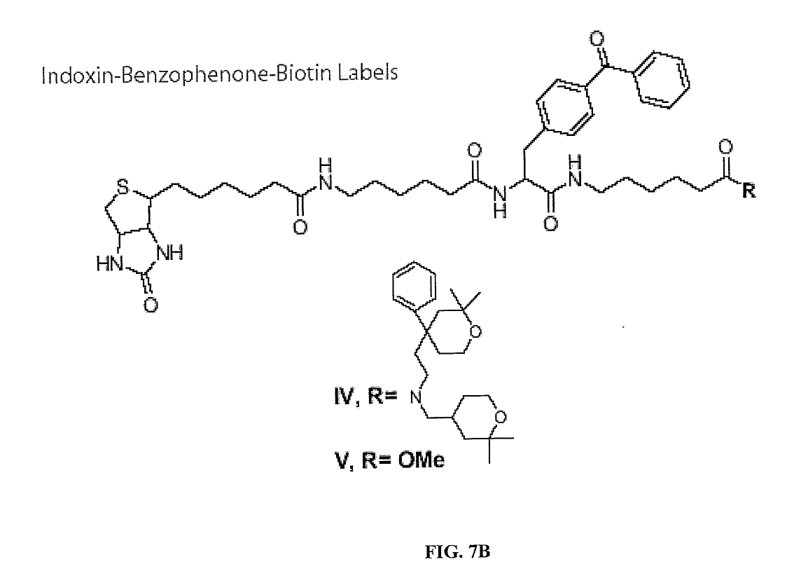

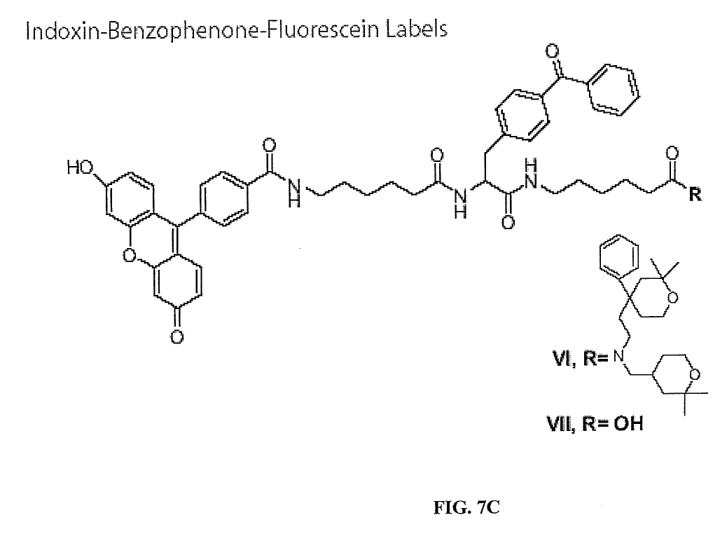

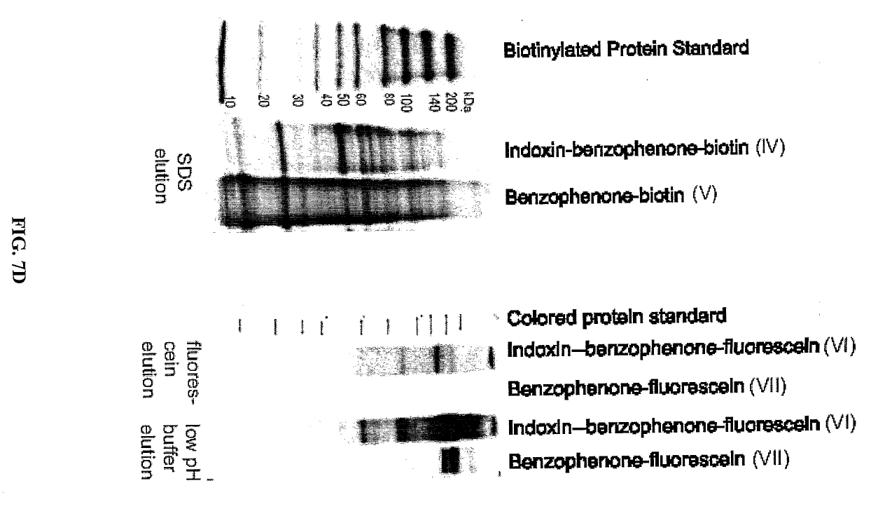

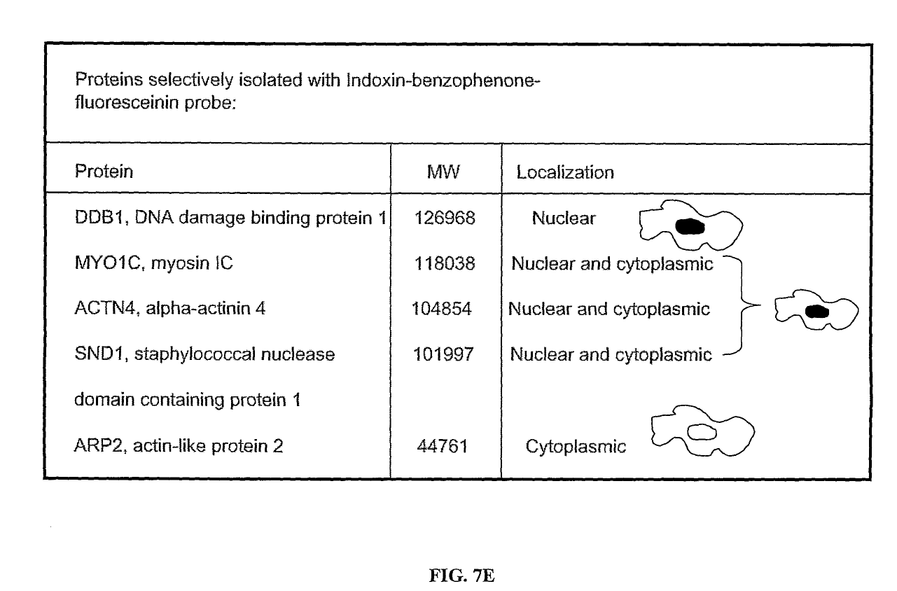

To analyze further indoxins' mechanisms of action, indoxin affinity probes were developed for protein target identification. Initially, a series of biotin-derivatized indoxin affinity probes I-III (FIGS. 7-8) were synthesized. Biotin provides a small, high-affinity tag for resin-based target protein pull-down (Hermanson, 1996), RKO-E6 cell lysates were prepared in a non-denaturing lysis buffer, incubated with the indoxin-biotin probes for 12-16 h at 4° C., and then cell lysates were passed over NeutrAvidin beads. The bound proteins were eluted by boiling NeutrAvidin beads in SDS page buffer and were resolved on Tris-glycine acrylamide gels. Protein was visualized via staining with Coomassie blue, silver, or SYPRO Ruby (Molecular Probes) stains. However, no selective protein targets were isolated using these probes. It was reasoned that since indoxins are active at micromolar concentrations and have no functional groups that could form a covalent bond with their protein targets, they provide insufficient affinity for direct target identification.

However, it was reasoned that incorporation of a photo-activatable functionality that allows for covalent bond formation between a small molecule affinity probe and its target protein may enhance the likelihood of protein target identification (Dorman and Prestwich, 2000; Weber and Beck-Sickinger, 1997). Benzophenone (BP) photo probes have become the group of choice for high-efficiency photo-covalent modifications of binding proteins in vitro (Dorman and Prestwich, 1994; Prestwich et al., 1997). p-benzoyl-L-phenylalanine (L-Bpa) was used to incorporate a benzophenone moiety in the biotin-tagged photo-reactive indoxin probe IV (FIG. 8). the biotin-tagged photo-reactive probe V lacking indoxin functionality was also prepared as a negative control (FIG. 8). The RKO-E6 cell lysates were incubated with the photo-reactive probes and irradiated with 350 nm ultraviolet light. Proteins cross-linked to these probes were purified using NeutrAvidin beads and eluted with SDS buffer. A number of non-specific bands were observed in both the indoxin-probe and control-probe pull-downs (FIG. 7D) that may be due to cross-linking to biotin-binding proteins.

It was reasoned that Biotin-induced cross-linking to the indoxin photoprobe could be substantially reduced if the biotin affinity tag were replaced with a non-natural affinity tag that does not have high affinity cellular targets. A number of antibodies have been developed against fluorescein, providing a convenient route for the resin-based isolation of protein targets. An indoxin-benzophenone-fluorescein photo-reactive probe VI was thus prepared (FIG. 8). Protein targets cross-linked to these probes were purified by immobilizing fluorescein on a protein-A-containing resin with an anti-fluorescein antibody. The proteins were specifically eluted with excess of fluorescein or with low pH buffer. The fluorescein affinity tag reduced non-specific cross-linking to the control probe VII (FIG. 7D). This fluoresceinated indoxin probe was used for a large-scale protein pull-down using both nuclear and cytoplasmic fractions of RKO-E6 cell lysates. Purified proteins were sequenced using liquid chromatography/tandem mass spectrometry (LC-MS/MS). Using the indoxin-fluorescein photolabel, a number of proteins were identified that had at least three corresponding peptide sequences in the MS data. In order to determine which of these proteins are specific for binding indoxin, identical experiments were performed for the control probe VII, lacking indoxin. Five proteins were identified that were selectively pulled-down with the indoxin probe (FIG. 7E) from the nuclear fraction.

To confirm these findings, the pull-down with the indoxin probe was repeated and two proteins were identified that were again selectively pulled down with indoxin A from the nuclear fraction. The actin-related proteins myosin 1C and ARP2 were both repeatedly pulled down with indoxin probes from the nuclear fraction, but were not present in the control pull-down experiments. Thus, a nuclear actin-related protein complex involving myosin 1c and ARP2 is a candidate target for mediating the effects of indoxins.

In this cell-based viability screen, several groups of compounds were identified that potentiate doxorubicin's lethality in E6-expressing tumor cells, thus overcoming E6-induced drug resistance. These compounds expand the very limited list of small organic molecules capable of inactivating E6-induced tumorigenicity and provide new avenues for developing adjuvant therapies.

Without wishing to be bound by theory, the proposed mechanism for increasing doxorubicin's lethality in E6-expressing tumor cells includes (i) upregulation of topoisomerase IIα and (ii) induction of S-phase arrest. Topoisomerase IIα is regulated in proliferation-dependent and cell-cycle-dependent manner (Chow and Ross, 1987; Larsen and Skladanowski, 1998). The level of topoisomerase IIα increases during S phase and reaches the highest concentration during late G₂/early M phase. In addition and somewhat independently, topoisomerase-IIα-mediated drug sensitivity markedly increases during S-phase (Chow and Ross, 1987). Thus, compounds that induce S-phase arrest can potentiate doxorubicin's lethality. Moreover, there is ample evidence that topoisomerase-targeting drug potency is directly related to the expression levels of the topoisomerases (Asano et al., 2005; Houlbrook et al., 1996; Kellner et al., 2002; Koshiyama et al., 2001; MacGrogan et al., 2003). Thus, compounds that directly up-regulate topoisomerase levels in tumor cells can potentiate topoisomerase-targeting drugs.

A group of compounds (indoxins) was identified that both upregulate topoisomerase IIα and induce S-phase arrest in RKO-E6 cells. Indoxin probes repeatedly and selectively pulled down myosin 1C (MYO1C) from the nuclear fraction of the RKO-E6 cells. The nuclear isoform of myosin 1C (NMI) shares more than 98% sequence homology with cytoplasmic myosin 1C (Pestic-Dragovich et al., 2000). Although LC-MS/MS analysis of the isolated sequences is not sufficient for distinguishing between the nuclear and cytoplasmic myosin 1C, the protein was isolated from the nuclear fraction of RKO-E6 cells and presumably is the nuclear isoform.

Given that indoxins upregulate topoisomerase IIα in both RKO and RKO-E6 cells, but only cause S-phase arrest in RKO-E6 cells, these two mechanisms can be dissociated. Thus, indoxins may acts as dual-action compounds that cause these two distinct effects (topoisomerase IIα upregulation and S-phase arrest) and that they both contribute to increased doxorubicin sensitivity. The Bliss Independence analysis supports this dual-mechanism hypothesis by demonstrating the RKO-E6 cells showed greater combination effect than RKO or HeLa cells. The ability of indoxins to inhibit nuclear myosin 1C could mediate topoisomerase IIα transcriptional upregulation, as nuclear myosin 1C has been linked to transcriptional control (see below). A cytosolic myosin target might mediate the S-phase arresting activity of indoxins. Both activities may contribute to the increased sensitivity of indoxin-treated cells to doxorubicin,

Nuclear myosin 1C co-localizes with RNA polymerase II and may affect transcription (Pestic-Dragovich et al., 2000). Nuclear actin and myosin 1 (NMI) are associated with rDNA and are required for RNA polymerase I transcription (Pestic-Dragovich et al., 2000). Depletion or inhibition of cellular myosin 1 or actin results in decreased nucleolar transcription, while overexpression of NMI amplifies pre-rRNA synthesis (Pestic-Dragovich et al., 2000). Thus, there is ample precedent for the hypothesis that nuclear myosin 1C could regulate topoisomerase IIα expression.

In summary, the most tractable mechanism for overcoming doxorubicin resistance is upregulation of the direct target of doxorubicin, topoisomerase IIα. In addition, induction of S-phase arrest potentiates the cytotoxicity of topoisomerase IIα-mediated DNA-damage. Each of these mechanisms provides modest potentiation of doxorubicin's lethality. For example, co-treatment of RKO-E6 cells with hydroxyurea and doxorubicin resulted in only modest upregulation of doxorubicin's lethality. However, together both these mechanisms result in a strong sensitization effect.

### Example 2

Small molecule screens were used to identify mechanisms and compounds for overcoming E6-induced resistance to apoptosis, as well as compounds that inhibit polyQ toxicity by reversing altered protein interactions of mutant huntingtin (htt) or by enhancing the protective effect of WT htt. For this purpose, a cell-based model with a defined genetic alteration was selected, for example expression of an N548 mutation of the htt protein. Small molecules are capable of altering the function of gene products in a conditional manner. Since the use of small molecules as therapeutics has been well established (compared to siRNA or cDNA), it is more likely that they can serve as leads for the development of new drug candidates. Synthetic compounds are also less prone to degradation via catabolic pathways and are more likely to be cell permeable and maintain biological activity in vivo.

Huntington's disease (HD) is an inherited neurodegenerative disorder with no available therapy. HD is characterized by selective loss of striatal neurons caused by polyglutamine (polyQ) expansion in the amino-terminus of the huntingtin (htt) protein. The mechanisms underlying toxicity of mutant htt are unresolved; both loss of wild type htt function and gain of function by mutant htt have been implicated in HD.

High-throughput, neuronal cell-based screens were developed in relation to Huntington's Disease. Assays exhibit mutant huntingtin (htt)-dependent toxicity that is found selectively in neuronal cells. These screens allowed for the identification of small molecules that prevent the toxicity of the expanded, polyglutamine-containing huntingtin protein in neuron-like cells in culture

Several HD models use exon1 of htt, since it produces a more severe phenotype. The N548 mutant was chosen for the primary screen because most protein-protein interactions of htt are in the region between N63 and N548 and studies have suggested that altered protein interactions of mutant htt may be pathogenic. Additionally, the protective effect of htt is in this region. Thus, the N548 mutant protein could help identify compounds that inhibit polyQ toxicity by reversing altered protein interactions of mutant htt or by enhancing the protective effect of WT htt. In an effort to find therapeutic agents and to illuminate mechanisms of mutant htt toxicity, a screen was conducted in a cell culture model of HD. This model has crucial features of HD, including striatal neuronal origin and a phenotype that recapitulates an important aspect of HD (cell death). Importantly, it can be used for screening in a high-throughput format.

### Methods

### Cell Lines and Cell Culture

Embryonic rat striatal neurons were conditionally immortalized by stably transfecting a temperature-sensitive SV40 large T-antigen (T-ag) to generate the ST14A cell line. ST14A cells proliferate at the permissive temperature (33° C.), but stop dividing at 39° C., as a result of T-ag degradation. ST14A cells were engineered to express WT or mutant polyQ in the context of N-terminal 63, N-terminal 548 or full length (FL) 3144 amino acids of human htt protein. As htt protein context modulates polyQ toxicity, testing these different length htt proteins could provide insight into the basis for context dependence. Both of these cell lines proliferate normally at the permissive temperature (33° C.) but upon serum deprivation at the non-permissive temperature (39° C.), T antigen is degraded, the cells differentiate into striatal neuronal cells, and undergo cell death over 48 to 72 h (Ehrlich M E, et al., Exp Neurol 2001, 167: 215-26; Rigamonti D, et al., J Neurosci 2000, 20: 3705-13; Weinelt S, et al., J Neurosci Res 2003, 71: 228-36; Torchiana E, et al., Neuroreport 1998, 9: 3823-7; Cattaneo E & Conti L, J Neurosci Res 1998, 53: 223-34; Cattaneo E, et al., J Biol Chem 1996, 271: 23374-9; Corti O, et al., Neuroreport 1996, 7: 1655-9). The rate of death is dependent on the expression of mutant or WT htt; there is enhancement of cell death in mutant htt expressing cells and retardation of cell death in WT htt-expressing cells compared to parental ST14A cells.

PC12 rat pheochromocytoma cells were transfected with exon-1 of the human huntingtin gene containing either 25 or 103 N-terminal polyQ repeats under an ecdysone inducible promoter. For enhanced stability the repeat portion consists of alternating CAG/CAA repeats. They were passaged in PC12 media (DMEM with 10% horse serum and 10% fetal bovine serum) at 37° C. in 9.5% CO₂. Mutant (mut) htt was induced by the ecdysone receptor agonist, tebufenozide. Following induction with tebufenozide, these cells express comparable levels of either mutant or non-mutant forms of huntingtin (Aiken C, et al., Neurobiol Dis 2004, 16:546-555).

### Compound Libraries

Approximately 47,000 compounds were screened. These included FDA-approved drugs and known biologically active compounds from NINDS (1040 compounds, Microsource Discovery Inc.) and ACL (2036 compounds) collections (Root D, et al., Chem Biol 2003, 10: 881-92), 20,000 synthetic compounds from a combinatorial library (Comgenex International, Inc), and 23,685 natural, semi-natural and drug-like compounds of unknown biological activity from diverse sources (Timtec, Interbioscreen, and Chembridge). All compounds were prepared as 4 mg/ml solutions in DMSO (dimethylsulfoxide) except NINDS compounds (10 mM solutions), in 384-well plates (Grenier, Part no. 781280). “Daughter plates” were prepared from stock plates by a 1:50 dilution in serum free DMEM (3 μl compound to 147 μl DMEM) in 384-well plates (Grenier, Part no. 781270).

### Screening and Data Analysis

ST14A Assay System: Calcein acetoxylmethyl ester (AM) (Molecular Probes, Eugene, Oreg.) is a non-fluorescent, membrane-permeable compound that is cleaved by intracellular esterases, which subsequently forms the anionic, fluorescent compound, calcein, that cannot permeate the cell membrane. Calcein can label viable cells because of the cleaving ability of active intracellular esterases, in addition to an intact plasma membrane preventing fluorescent calcein from exiting cells (Wang X M, et al., Human Immuno 1993, 37: 264-70). A fluorescence viability assay, that is based on conversion of a non-fluorescent substrate (calcein AM) to a fluorescent product by nonspecific esterases in live cells, was employed to monitor cell death in wild type (wt) ST14A-Htt and mut ST14A-Htt cell lines. Cell death is indicated by a decrease in fluorescence. In 384-well plates (Costar 3712), 1500 cells/well were seeded in 57 μl of DMEM media with 0.5% IFS. 3 μl of each compound was transferred from daughter plates to triplicate assay plates for a final assay concentration of 4 μg/ml in 0.1% DMSO (10 μM for NINDS compounds). Cells were incubated at 33° C. for 3 h to enhance attachment and then shifted to 39° C. (with 5% CO₂). After 3 days (d), cells were washed 10 times with phosphate buffered saline (PBS) leaving 20 μl residual PBS per well, and 20 μl of 2 μg/ml calcein AM (Molecular probes) in PBS was added per well. Cells were incubated at room temperature for 4 h and fluorescence (ex 485/em 535 nm) intensity was recorded with a read time of 0.2 seconds per well using a fusion plate reader (Packard). The fluorescence intensity in each well was normalized to the median of each plate. The median normalized fluorescence of each triplicate assay wells was determined. A well with more than 50% increase in intensity above the median signal intensity was considered a “hit.”

For the PC12 assay, 7500 cells/well were plated in 384-well plates in 57 μl of PC12 medium with tebufenozide (1 μM). Compounds (3 μl) from daughter plates were added to the cells and incubated at 37° C. After 48 h, 20 μl of 40% Alamar Blue (Biosource, CA) in PC12 media was added per well and cells incubated for 12 h at 37° C. Cell viability was assayed by measuring of Alamar blue reduction (ex 530/em 590 nm). Fluorescence intensity was determined using a Packard Fusion plate reader with an excitation filter centered on 535 nm and an emission filter centered on 590 nm. Average percentage inhibition at each concentration was calculated. The Alamar Blue assay does not involve washing the cells.

### Western Blot Analysis

Cells were lysed in Lysis Buffer (50 mM HEPES KOH, pH 7.4; 40 mM NaCl; 2 mM EDTA; 0.5% Triton X-100; 1.5 mM Na₃VO₄; 50 mM NaF; 10 mM sodium pyrophosphate; 10 mM sodium β-glycerophosphate; and a protease inhibitor tablet (Roche, Indianapolis, Ind.)). Protein content was quantified using a BioRad protein assay reagent (Hercules, Calif.). Equal amounts of protein were resolved on a 16% SDS-polyacrylamide gel. The electrophoresed proteins were transferred onto a PVDF membrane, blocked with 5% milk, and incubated with an anti-T-ag polyclonal antibody (Santa Cruz Biotechnology, Pab 108; Santa Cruz, Calif.) overnight at 4° C. The membrane was then incubated with anti-rabbit-HRP (Santa Cruz Biotechnology; Santa Cruz, Calif.) for 1 hr and developed with an enhanced chemiluminescence mixture (Perkin Elmer; Wellesley, Mass.). Blots were later stripped, blocked, and re-probed with an anti-p-tubulin monoclonal primary antibody (Sigma-Aldrich, clone TUB2.1) and a goat anti-mouse HRP secondary antibody (Santa Cruz Biotechnology; Santa Cruz, Calif.).

### Indirect Immunofluorescence

Cells were grown on glass coverslips in 10% IFS-containing media, treated with compounds and fixed in acetone/methanol (1:1 vol/vol). β-tubulin was detected using a mouse monoclonal antibody (clone TUB2.1, Sigma-Aldrich) followed by a rhodamine-conjugated goat anti-mouse antibody (Jackson Laboratory; Bar Harbor, Me.). Cells were viewed under a fluorescent microscope (ex 530/em 595 nm).

### Caspase Assay

Caspase inhibitors purportedly rescue polyQ-mediated toxicity in several model systems, including the one described in this invention (Chen M, et al., Nat Med 2000, 6: 797-801; Kim M, et al., J Neurosci 1999, 19: 964-73; Rigamonti D, et al., J Biol Chem 2001, 276: 14545-8; Wellington C L & Hayden M R, Clin Genet 2000, 57: 1-10; Ellerby L M, et al., J Neurochem 1999, 72: 185-95). As a control, the ability of the general caspase inhibitor BOC-D-FMK was tested to rescue Htt-Q 103-mediated cell death in this assay system. The addition of 50 μM BOC-D-fmk to Htt-Q103 cells at the time of tebufenozide induction resulted in a complete (100+%) rescue of the Htt-Q103-induced cytotoxicity.

Caspase activity was measured using a fluorogenic assay (Biovision Inc. CA), based on cleavage of AFC (7-amino-4-trifluoromethyl coumarin) from specific AFC-conjugated peptide substrates by activated caspases. Each cell line was seeded at 10⁶ cells/plate, incubated overnight at 33° C., and then incubated for 6 h at 39° C. in 0.5% IFS containing medium with or without BOC-D-fmk (50 μM). Four plates/sample were harvested in lysis buffer provided by the manufacturer. Peptide substrates were added to the cell lysate or to lysis buffer (control), incubated at 37° C. for 2 h, and fluorescence (ex 355/em 510 nm) measured on a plate reader (Perkin Elmer Victor³). Fluorescence intensities of controls were subtracted from sample intensity and the resulting value normalized to protein in each sample (Bradford Assay from BioRad; Hercules, Calif.).

### Neuronal Survival Assay

One hundred synchronized L1 worms (pqe-1; Htn-Q150) (Faber P., et al, Proc Natl Acad Sci 99: 17131-6; Wood W., Cold Spring Harbor Laboratory, NY) were added to 5 wells (20 worms/well). Each well contained 50 μl food suspension (6.6 O.D.) pre-mixed with compound or DMSO in a 96-well plate. Worms were incubated for 2 d at 15° C., washed in S-media, immobilized with 5 mM sodium azide on a microscopic glass slide and GFP fluorescence was examined using an Axoplan2 fluorescence microscope (ex 485/em 535 nm). Live (GFP positive) Anterior Sensory Horn (ASH) neurons were counted in at least 50 worms (100 neurons). Data were subjected to a two-tailed Student t-test.

### Equations

CV(coefficient of variation)=100*(SD/Mean); (I)

Z′ factor=1−(3*SD₁+3*SD₂)/(Mean₁−Mean₂) (II)

1=control, 2=positive outcome. SD=standard deviation.

### Results

In order to identify candidate compound leads and understand underlying mechanisms of toxicity, a high-throughput assay was developed in a striatal cell culture model of HD. 47,000 compounds were screened and small molecules were identified that selectively alleviate mutant htt toxicity but do not prevent cell death in general. Since htt protein length (context) modulates polyQ toxicity by unknown mechanisms, the identified small molecules were tested for activity in cell lines expressing different length htt protein (N-terminal 63 amino acids, N-terminal 548 amino acids and full length) with expanded (mutant) or unexpanded (wild type) polyQ. Three categories of inhibitors of mutant htt-induced toxicity were identified: (i) compounds that prevent polyQ-enhanced cell death independent of htt context, (ii) compounds that prevent cell death in a htt-length-dependent manner and (iii) compounds that enhance the cell survival function of wild type htt.

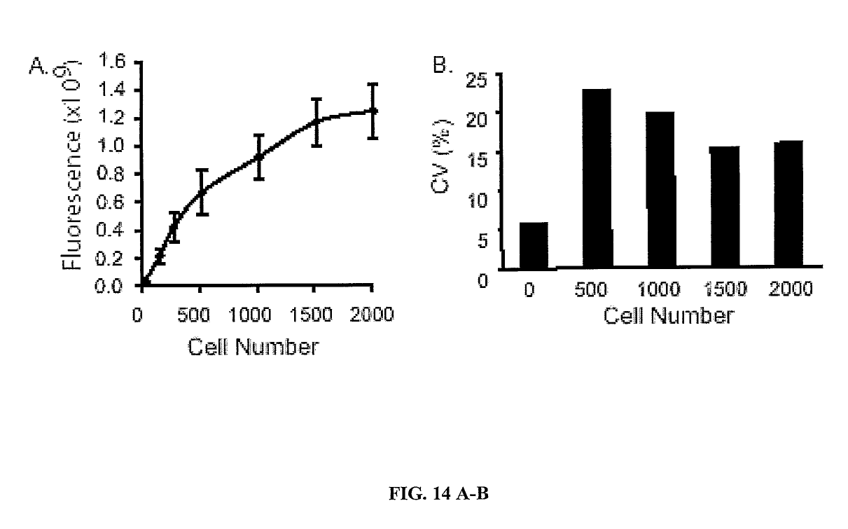

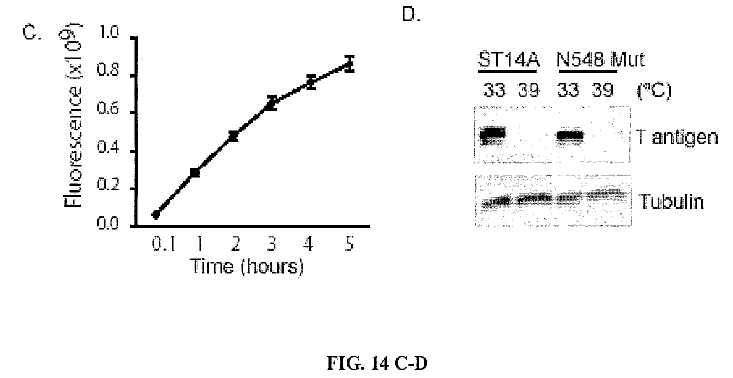

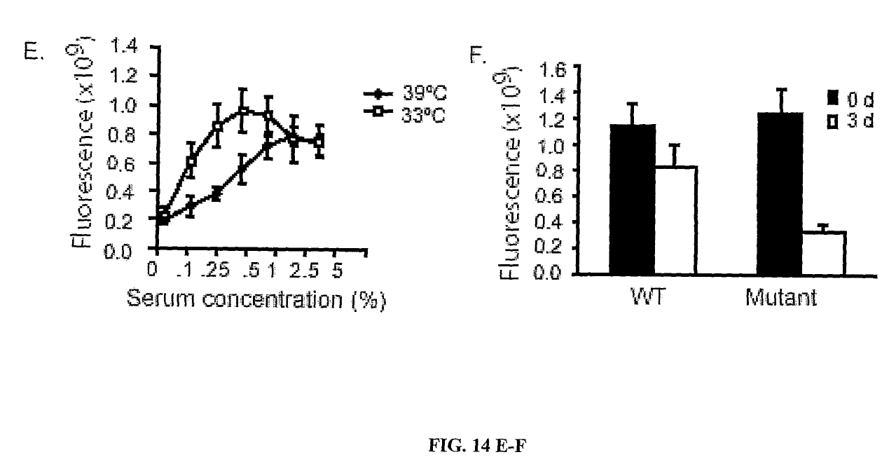

Assay Development: An assay was developed for detecting mutant htt-induced cell death in ST14A cells in a 384-well plate format. A calcein AM-based method was used to detect cell viability, and optimized the cell number and time course for this method. Calcein AM is a cell permeable non-fluorescent dye that is cleaved by cellular esterases to generate fluorescent calcein, and is retained by live cells (Wang X, et al Human Immunology, 1993, 37: 264-70). A cell number titration was performed to determine the cell density at which calcein fluorescence was not saturated and the coefficient of variation (CV) was low (FIG. 14A-B). A low CV is critical for a high-throughput assay as it decreases noise and enhances sensitivity. Based on this analysis, 1500 cells/well were chosen. Next, the kinetics of the fluorescence signal were determined. Calcein signal increased linearly over 4 h (FIG. 14C), giving a time window for performing the assay in a high-throughput manner. Thus, with a read time of 4 minutes for a 384-well plate, about 60 plates could be processed serially within 4 h of calcein AM addition.

Three key features of the ST14A model were confirmed: 1) temperature-dependent degradation of T-ag, 2) cell death upon serum deprivation, and 3) the protective effect of WT htt. Western blotting for T-ag (FIG. 14D) showed that a shift to 39° C. decreased T-ag protein within 6 h in both parental and mutant cell lines, consistent with degradation of T-ag at 39° C. Next, the serum concentrations that induce cell death in these cells were determined. Cells were seeded in decreasing serum (IFS) concentrations (5 to 0%), and cell viability was assayed after 3 d using the calcein AM assay (FIG. 14E). N548 mutant cells at 33° C. and 39° C. had similar viability in serum concentrations above 1% suggesting that higher temperature alone did not affect cell viability. Serum concentrations between 0.25% and 1% selectively decreased cell viability in differentiated (39° C.) mutant cells while lower serum concentration affected viability of undifferentiated cells (33° C.) as well. For inducing cell death in the screen, a concentration of 0.5% serum was thus chosen. Experiments from this study confirmed that in 0.5% serum, WT N548 expressing cells were protected relative to mutant cells (FIG. 14F). Additionally, serum deprivation (0.5% IFS) decreased viability in the parental ST14A cells to levels between WT and mutant htt cell lines allowing for use of this cell line to identify compounds that prevent cell death in a non-selective manner.

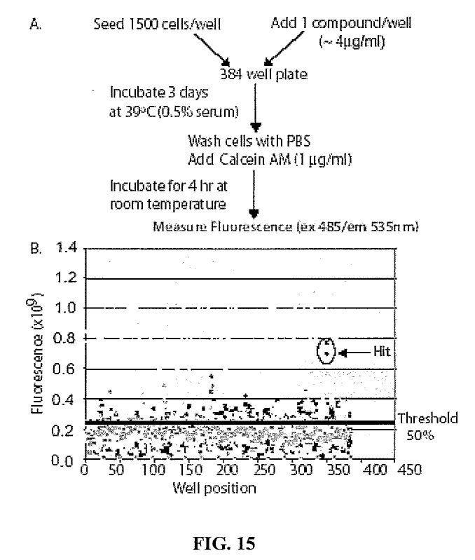

Assay optimization for high-throughput screening (HTS): The Z′ factor is a measure of the quality of a high-throughput assay (Zhang J, et al J Biomolec Screen 1999, 4: 67-83). A Z′ value of greater than 0 is required for a usable assay, with a maximum value of 1.0 for an ideal assay. For the assay, calcein fluorescence was used on the day of seeding to represent 100% rescue. The difference in calcein fluorescence signal on the day of seeding and after different number of days under serum deprivation was determined and used to calculate the Z′ factor. 3 days after serum deprivation, the assay had a Z′ factor between 0.1 and 0.25; allowing HTS. About 47,000 compounds were assayed in a 384-well plate format in triplicate to enhance the reliability of the primary screen. Any compound that increased calcein fluorescence 50% above the median plate fluorescence was considered a “hit” (FIGS. 15A-B). All hits were confirmed by retesting the compounds in dose-response titration assays in triplicate in three independent experiments. Based on these criteria, 50 compounds were identified that prevented cell death in N548 mutant htt cells.

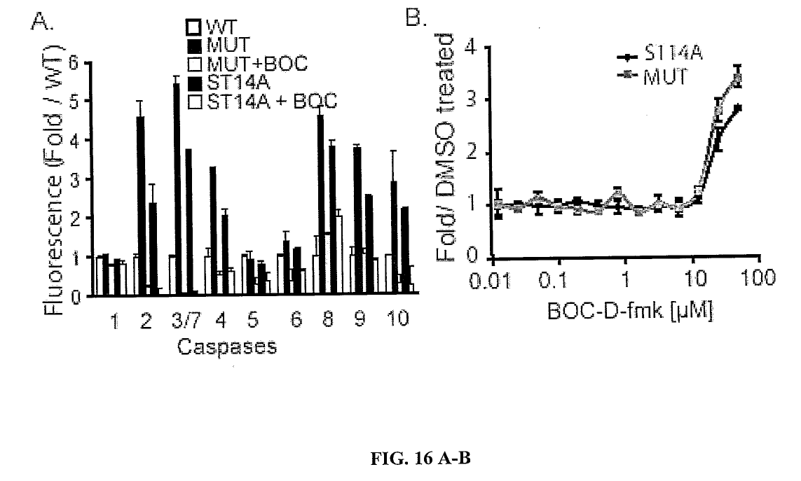

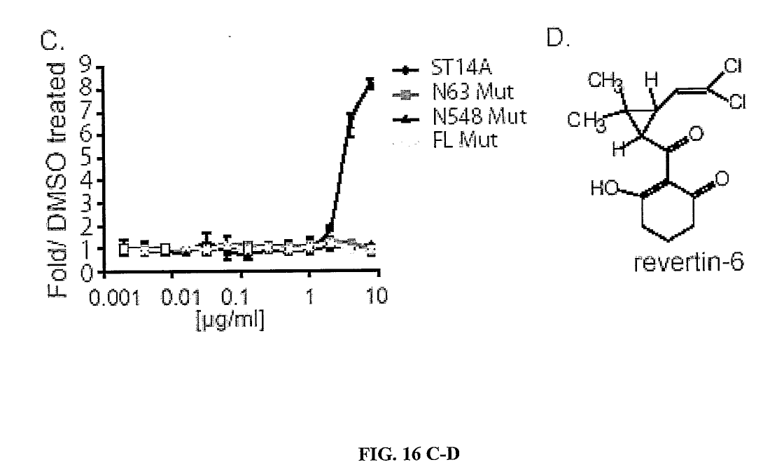

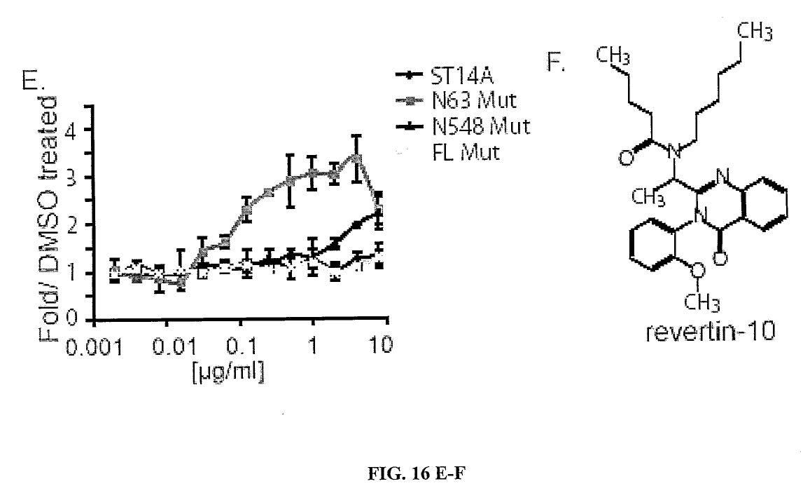

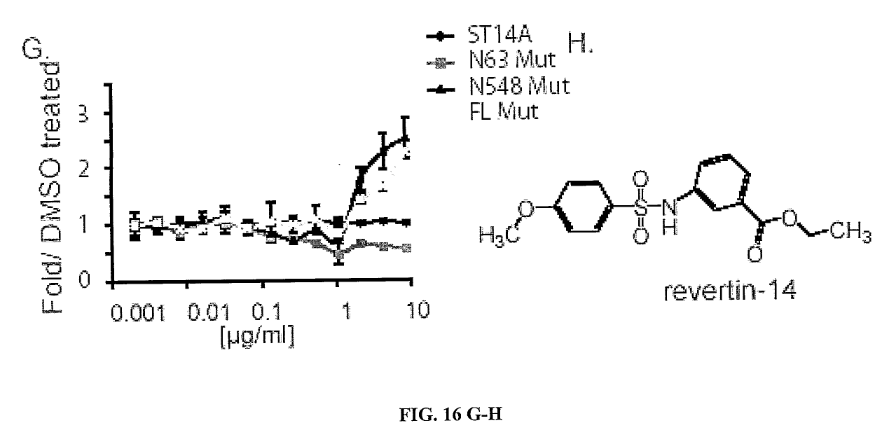

Identification of selective inhibitors of mutant-htt toxicity: As a first step towards probing mechanisms of action of these compounds, it was tested whether compounds selectively targeted pathways that are perturbed by mutant htt or if they suppressed a general death mechanism. Caspase activation was tested to see if it contributed to cell death in this model, since caspase-dependent pathways have been broadly implicated in cell death and in HD (Thornberry, N. and Y. Lazebnik, Science 1998, 281:1312-6; Sanchez Mejia, R and R. Friedlander Neuroscientist 2001, 7: 480-9; Hickey M. and M. Chesselet, Prog Neuropsychopharmacol Biol Psych 2003, 27: 255-65). Caspase activation was monitored via measuring the cleavage of specific fluorogenic caspase substrates. Enhanced caspase activation was observed in mutant htt cells and inhibition of caspase activation in WT htt cells compared to ST14A cells (FIG. 16A). The decreased caspase activation in WT N548 cells was consistent with relative protection of these cells from cell death. Additionally, BOC-D-fmk, a pan-caspase inhibitor (Deas O, et al. J Immunol 1998, 161: 3375-8.3), prevented caspase activation and rescued cell death in both ST14A and mutant N548 cells (FIG. 16A-B). These results suggest that caspase pathways are not specific for mutant htt-enhanced cell death but are a consequence of serum deprivation; though, mutant htt may enhance the overall levels of caspase activity. Thus, the ability of a compound to rescue cell death in ST14A cells could be used as a selectivity filter to differentiate compounds that were selective for pathways selective for mutant htt induced cell death. The selectivity of rescue by a compound was determined by assessing rescue of cell death in parental ST14A cells. A number of compounds with known biological mechanisms were identified as non-selective protective agents by this counter screen (Table I below). Some of these compounds are FDA-approved drugs and thus may have therapeutic potential. Additionally, 40 compounds selectively suppressed cell death in mutant htt-expressing cells (all structures are in Table 3). These compounds were named “revertins“(reversal of mutant huntingtin toxicity). Revertins may modulate pathways affecting cell viability that are perturbed by mutant htt.

TABLE 1
Nonselective hits

| Compound | Biological mechanism | EC50 (μM) | Activity | TC50* | FDA approved |
|---|---|---|---|---|---|
| BOC-D-fmk | Pan-Caspase inhibitor | 25 | 3 | ND(80) | No |
| Budesonide | Glucocorticoid agonist | 0.5 | 2 | ND | Yes |
| Clofibrate | PPAR alpha agonist | 1 | 2 | ND | Yes |
| Tretinoin | Retinoid receptor agonist | 0.6 | 2.5 | ND | Yes |
| Flufenamic Acid | Cyclooxygenase inhibitor | 5 | 1.5 | 40 | No |
| Prostaglandin E2 | G-protein coupled receptor signaling | 0.1 | 2 | ND | No |
| Zaprinast | cGMP Phosphodiesterase | 5 | 1.5 | ND | No |
| Tetrahydrobiopterin | Cofactor | 0.5 | 2.5 | ND | No |
| Homidium Bromide | DNA intercalator | 10 | 1.5 | 15 | No |
| 2-NPPB | Chloride channel blocker | 10 | 1.5 | 40 | No |

*The maximum compound concentration tested was 80 μM,
ND: no toxicity detected

Identification of htt length-dependent inhibitors of mutant htt toxicity: Previous work suggested that htt protein length (context) affects polyQ toxicity (Yu Z, et al., J Neurosci 2003, 23: 2193-202; Chan E, et al, Hum Mol Genet 2002, 11: 1939-51) but the mechanisms for context dependence are not known. Also, it is not clear if polyQ in different htt contexts causes toxicity by the same or by different mechanisms.

If all compounds that are active in one context (e.g. N548) are also active in others contexts, one could infer that polyQ triggers toxicity by common pathways in each case. However, if some compounds affect viability only in specific htt protein contexts, it would suggest that distinct pathways are perturbed by polyQ in each htt context. Such results would also suggest that these compounds act by distinct mechanisms.

To answer this question, the ability of compounds to rescue cell death in cell lines expressing mutant or the normal length glutamine stretches in the context of N63, N548 or FL htt was tested. The panel of cell lines that a compound prevented cell death in was referred to as the compound's “selectivity profile”. Based on the results of this testing, selective compounds were grouped into 3 selectivity profile classes (Table 2).

TABLE 2
Classes of selective hits

| | | Mutant | | | Wild Type | | | no. of |
|---|---|---|---|---|---|---|---|---|
| Class | Hit Length | N63 | N548 | FL | N63 | N548 | FL | cpds |
| Class I | N63, N548, FL | + | + | + | − | − | − | 5 |
| Class II A | N548 | − | + | − | − | − | − | 1 |
| Class II B | N63, N548 | + | + | − | − | − | − | 5 |
| Class II C | N548, FL | − | + | + | − | − | − | 6 |
| Class III | Mutant + WT | | | | | | | 19* |

| | | | | | | | |
|---|---|---|---|---|---|---|---|
| (Microtubule inhibitors) | − | + | + | − | + | − | 5 |

*These 19 include 5 microtubule inhibitors while the remaining compounds have unknown/different mechanisms and different selectivity profiles

Compounds that suppressed death in the context of all mutant htt proteins (N63, N548 and FL) were designated as class I. Compounds that suppressed cell death in a htt-length-dependent manner were designated as class II. Class II compounds were subdivided into 3 subclasses based on their efficacy in different length mutant htt-expressing cells, namely; compounds that rescued cell death in N548-mutant-expressing cells only (class IIA), compounds that rescued cell death in N63 and N548 mutant (class IIB) and compounds that rescued cell death in N548 mutant and FL mutant but not N63 mutant (class IIC). Dose response curves of the rescue for one representative compound for each class (class IIA, IIB and IIC), along with their structures, are shown in FIG. 16C-H. Finally, compounds that suppressed cell death in both mutant htt and WT htt-expressing cells (at least one cell line expressing mutant or WT htt in any context) were designated as class III. Based on this analysis, compounds were identified that utilize at least 5 distinct mechanisms to rescue mutant htt toxicity.

TABLE 3
Structures, efficacy and effective concentrations of various
classes of selective hits in N548 mutant and PC12 HD

| | | N548 | | | | | Catalog |
|---|---|---|---|---|---|---|---|
| Compound | Structure | Mut^{a} | EC₅₀^{b} | PC12^{c} | EC₅₀ | Source^{d} | no. |
| Class I | | | | | | | |
| revertin-1a | 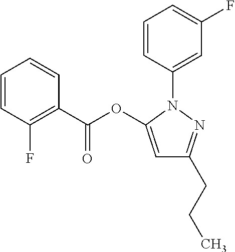 | 2.5 | 4 | 21 | 50 | CGX | 0640215 |
| revertin-1b | 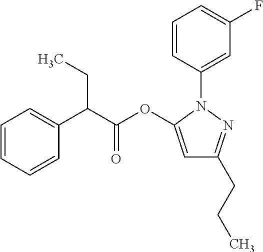 | 25 | 4 | — | n.a. | CGX | 0640213 |
| revertin-1c | 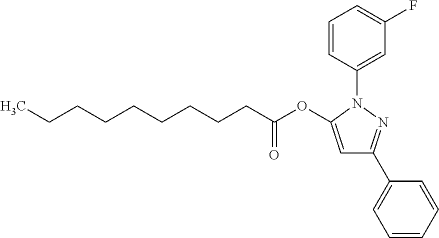 | 2.5 | 4 | 23 | 40 | CGX | 0634621 |
| revertin-2 | 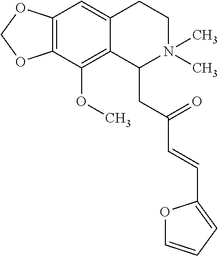 | 4 | 3 | 34 | 13 | IBS | 1N-27982 |
| revertin-3 | 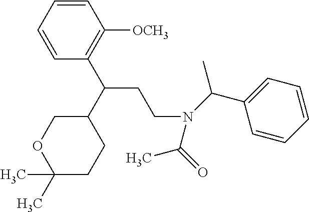 | 4 | 2 | — | n.a. | IBS | 1N-21372 |
| Class II A | | | | | | | |
| revertin-4 | 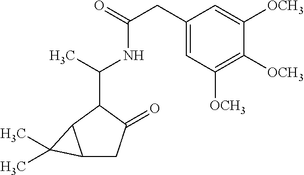 | 1.5 | 4 | — | n.a. | IBS | 1N-27238 |
| revertin-5 | 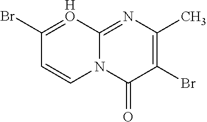 | 1.5 | 0.5 | — | n.a. | CB | 5785879 |
| revertin-6 | 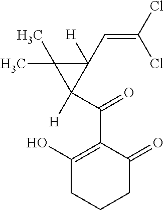 | 8 | 3 | — | n.a. | IBS | 1N-32243 |
| Class II B | | | | | | | |
| revertin-7 | 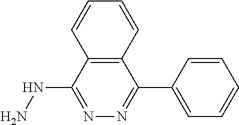 | 2 | 1 | 18 | 2 | CB | 5753607 |
| revertin-8 | 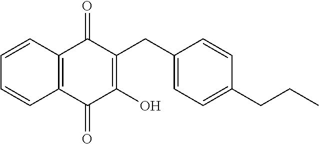 | 3 | 2 | 15 | 2 | CB | 5719309 |
| revertin-9 | 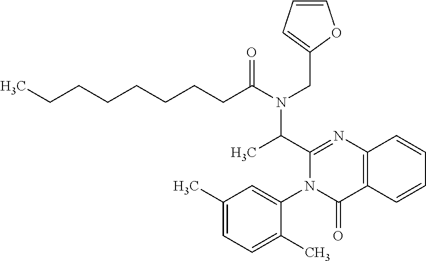 | 2 | 1 | 25 | 25 | CGX | 0528585 |
| revertin-10 | 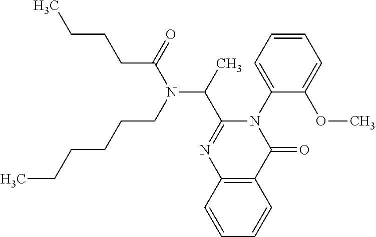 | 1.5 | 4 | — | n.a. | CGX | 0532894 |
| revertin-11 | 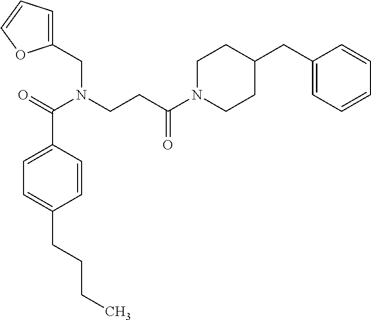 | 2.5 | 2 | 27 | 30 | CGX | 0509488 |
| revertin-12 | 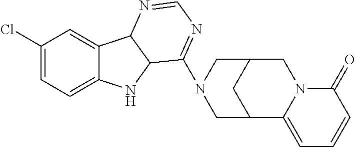 | 1.5 | 3 | — | n.a | IBS | 1N-31243 |
| revertin-13 | 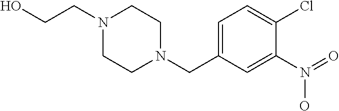 | 1.5 | 5 | — | n.a. | CB | 5347986 |
| Class IIC | | | | | | | |
| revertin-14 | 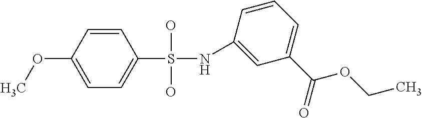 | 2.5 | 10 | — | n.a. | CB | 6617574 |
| revertin-15 | 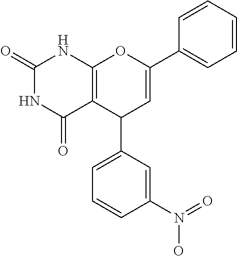 | 3 | 1 | — | n.a. | Timtec | ST2007542 |
| revertin-16 | 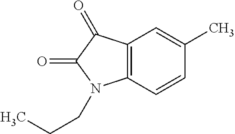 | 2 | 1 | — | n.a. | CB | 6137105 |
| revertin-17 | 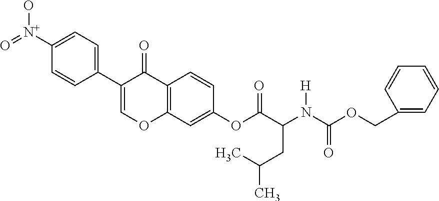 | 1.5 | 4 | — | n.a. | Timtec | ST222547 |
| revertin-18 | 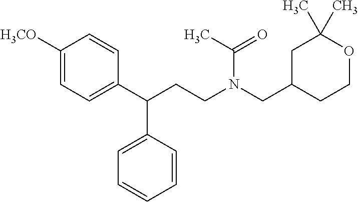 | 1.5 | 4 | — | n.a. | IBS | 1N-30665 |
| revertin-19 | 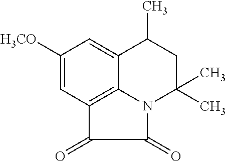 | 2 | 4 | — | n.a. | IBS | 1N-31830 |
| Class III | | | | | | | |
| revertin-20 | 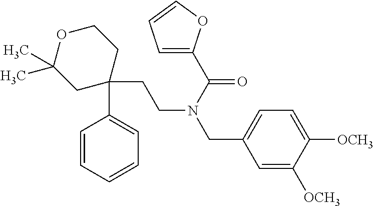 | 2 | 8 | — | n.a. | IBS | 1N-12255 |
| revertin-21 | 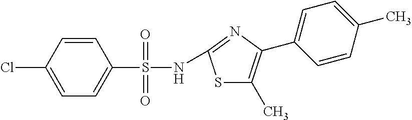 | 3 | 4 | — | n.a. | CB | 6655826 |
| revertin-22 | 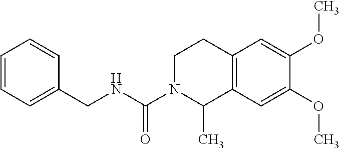 | 2.5 | 1 | — | n.a. | CGX | 0602488 |
| revertin-23 | 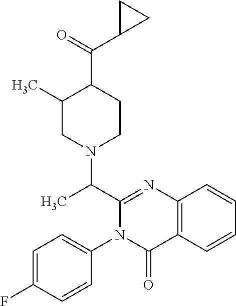 | 2.5 | 2 | — | n.a. | CGX | 0451517 |
| revertin-24 | 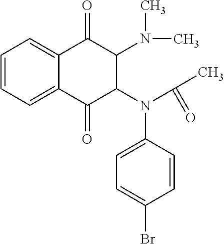 | 25 | 2 | — | n.a. | CB | 5543301 |
| revertin-25 | 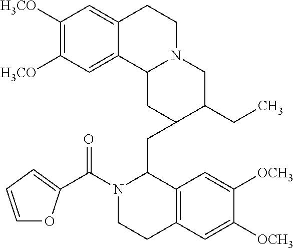 | 4 | 3 | — | n.a. | IBS | 1N-23587 |
| revertin-26 | 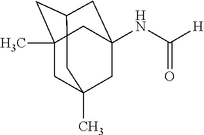 | 1.5 | 5 | — | n.a. | CB | 5347772 |
| revertin-27 | 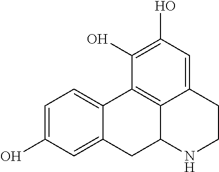 | 3 | 4 | — | n.a. | Timtec | ST2007542 |
| revertin-28 | 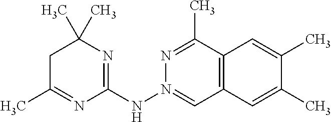 | 2.5 | 2 | — | n.a. | CB | 5658173 |
| revertin-29^{†} | 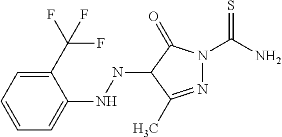 | 3 | 2 | — | n.a. | CB | 5711134 |
| revertin-30^{†} | 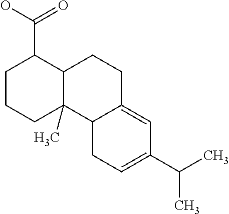 | 4 | 2 | — | n.a. | IBS | 1N-12989 |
| revertin-31^{†} | 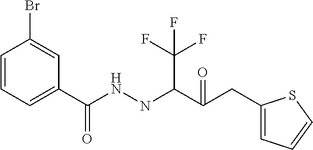 | 4 | 2 | — | n.a. | CB | 5649218 |
| Colchicine | 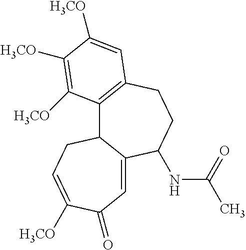 | 2.5 | 40 nM | — | n.a. | Sigma | C 3915 |
| Podophyllotoxin | 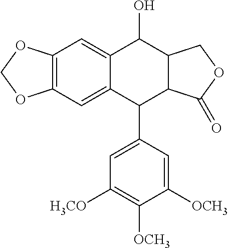 | 2.5 | 25 nM | — | n.a. | Sigma | P 4405 |
| Vincristine | 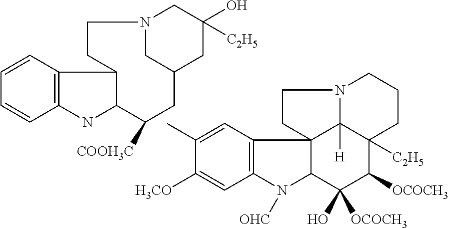 | 2.5 | 40 nM | — | n.a. | Sigma | V 8879 |
| Antimycin A | 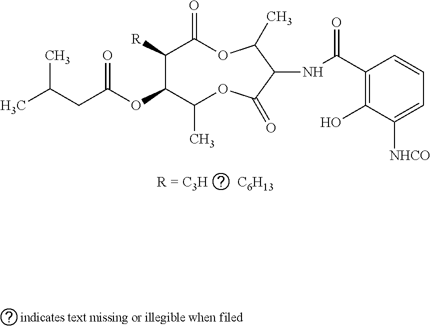 | 3 | 5 μM | — | n.a. | Sigma | A 8674 |
| Rotenone | 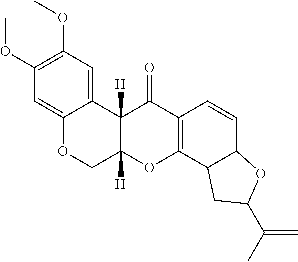 | 2.5 | 5 μM | — | n.a. | Sigma | R 8875 |
| Nonactin | 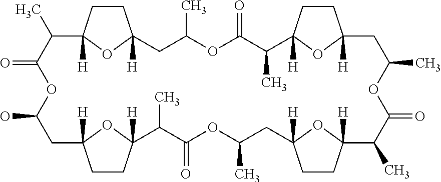 | 2.5 | 0.6 μM | — | n.a. | Sigma | 09877 |
| Valinomycin A | 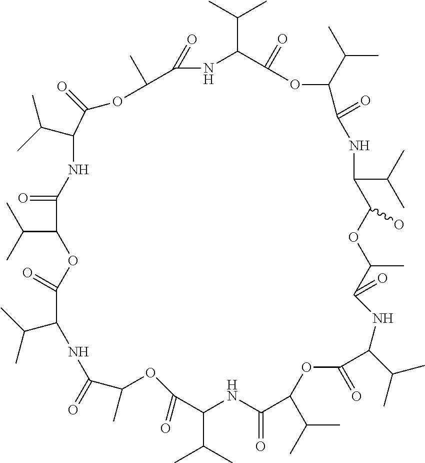 | 2.5 | 150 nM | — | n.a. | Sigma | V 0627 |

^{a}Max-maximum rescue expressed as fold over DMSO treated N548 mutant cells.
^{b}EC 50 (μg/ml) was based on activity in N548 mutant cells:
^{c}PC12 rescue: percent increase in viability in compound treated cells above DMSO treated PC12 expressing Q103 exon 1 htt.
^{d}CB-Chembridge; CGX-Comgenex; IBS-Interbioscreen.
^{†}relatively selective compounds-these were weakly active in parental ST14 A but more efficacious in mutant N548 cells.
n.a.-not applicable as the compounds did not show any activity in the assay.
Class I-poly Q selective compounds.
Class II-hit length selective compounds.
Class III-potential WT htt-protection enhancing compounds.

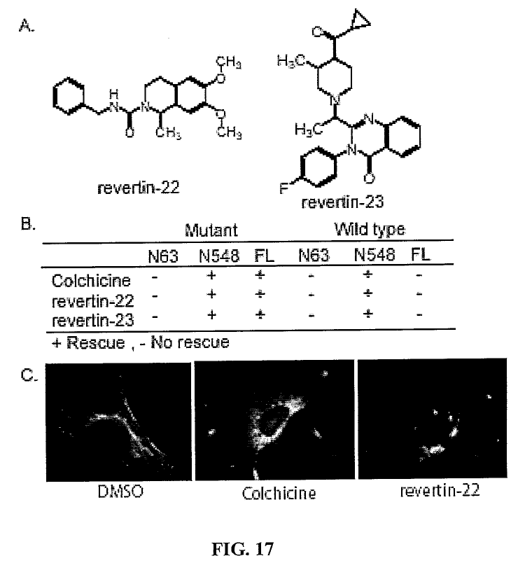

Identification of microtubule inhibitors based on selectivity profiling: If the classification of compounds based on context dependent rescue is meaningful, one could hypothesize that compounds in the same class are likely to act on the same pathway, but on a different pathway than compounds in another class. In this screen, microtubule inhibitors (MTIs) such as colchicine (Jordan A, et al, Med Res Rev 1998, 18: 259-96) that depolymerize microtubules, rescued cell death in three htt expressing cell lines (FIG. 17B). Two compounds, revertin-22 and revertin-23 (FIG. 17A), shared the selectivity profile with MTIs, but were structurally distinct from known MTIs ((Jordan A, et al, Med Res Rev 1998, 18: 259-96) and Table 1). Thus, these compounds may act as MTIs, explaining their selectivity profile. Using immunofluorescence for β-tubulin, the ability of these compounds to disrupt microtubules was confirmed (data shown for revertin-22, FIG. 17C). Further, the dose response of cell death rescue by these compounds paralleled that for MT disruption, suggesting that MT disruption was the mechanism by which these compounds rescued cell death. The mechanism of selective death rescue by MTI's is currently being studied. Thus, selectivity profiling can be used to identify the mechanism of action of compounds if compounds with known mechanisms of action share their selectivity profile.

Secondary screening. In order for a compound to be an attractive lead compound, it is important to show efficacy in more than one HD model; such compounds would likely affect a conserved mechanism of htt toxicity. The identified hits for rescue of HD phenotypes were tested in diverse models: a neuronal cell culture model (PC12), as well as yeast and worm HD models.

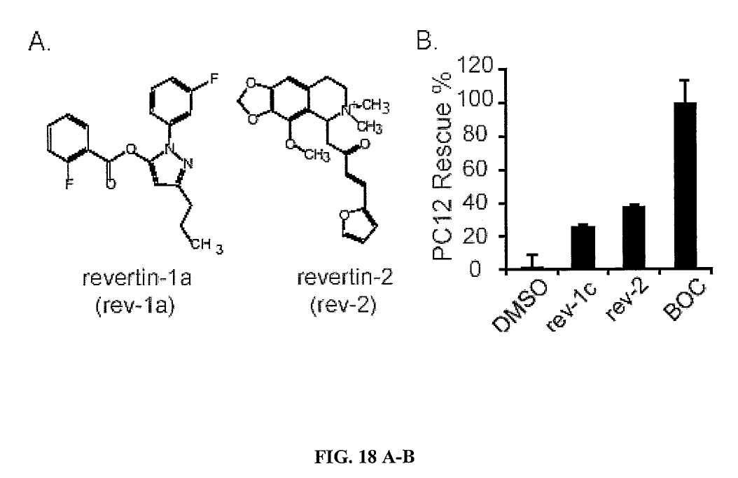

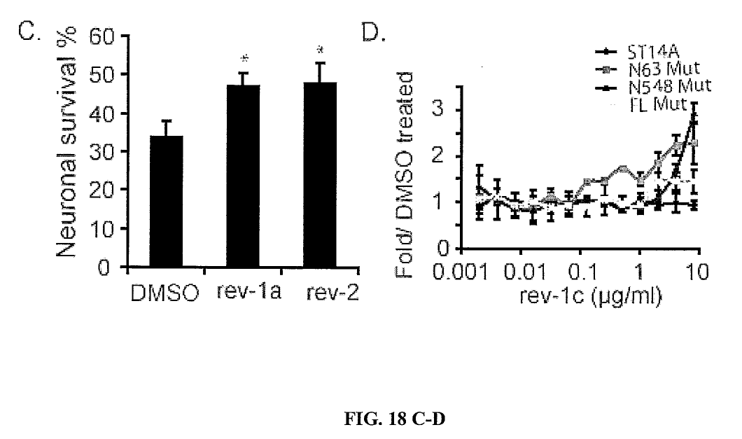

PC12 HD Model. An inducible model of mutant htt toxicity in PC12 cells was optimized for HTS (Aiken C, et al., Neurobiol Dis 2004, 16: 546-55; B. Hoffstrom and B. R. Stockwell, unpublished data). Induction of mutant htt transgene (exon 1 with 103Q) caused cell death over 48-72 h. Cell viability was assayed using Alamar Blue (Nociari M, et al, J Immunol Methods 1998, 213: 157-67). Consistent with an earlier report, BOC-D-fmk completely rescued cell death in this model (Aiken C, et al., Neurobiol Dis 2004, 16: 546-55). Of all the compounds tested, a few were active in this model (Table 3 and FIG. 18B).

Yeast HD Model. Galactose 2% inducible expression of an exonl htt transgene with 72 glutamines (Q72) reduced yeast growth compared to uninduced or Q25 expressing yeast cells (Krobitsch S and S Lindquist, Proc Natl Acad Sci USA 2000, 97: 1589-94). Compounds were tested for their ability to rescue the growth of Q72 htt yeast cells. None of the compounds reproducibly rescued yeast (Q72) growth ( ).

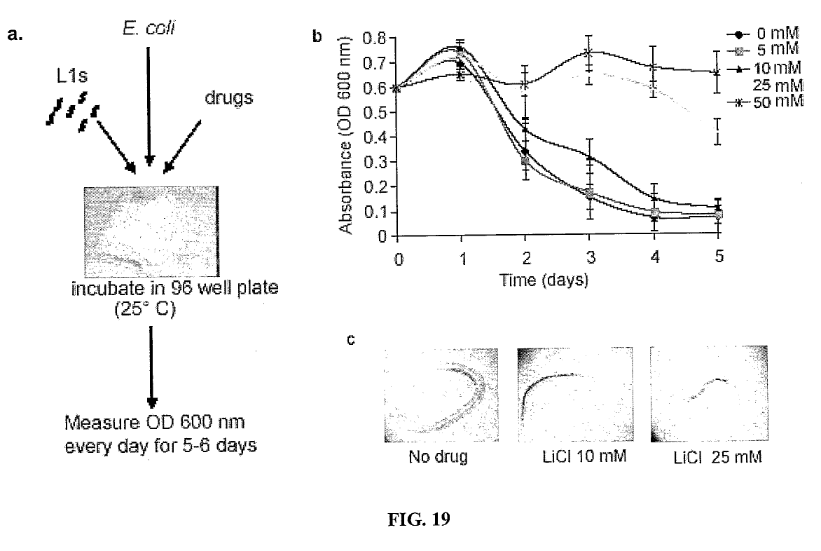

Worm HD Model. A C. elegans model of HD was optimized for testing compounds. In this model, larval ASH neurons that express mutant htt in a poly Q enhancer-1 mutant background, die over 2-3 days after hatching (Faber P, et al, Proc Natl Acad Sci USA 2002, 99: 17131-6). Neuronal death was monitored by loss of GFP expression in ASH neurons. Since the effective concentration of compounds in worms can vary widely from those in cell culture, concentrations to test in worms were based on an assay (FIG. 19). The effective concentrations in worms for all compounds were determined and tested for rescue of neuronal cell death. One compound was identified (rev-2) (FIG. 18A) that rescued ASH neuronal cell death in the worm HD model and was designated revertin-2 (FIG. 18C) This compound was protective in all three mutant htt versions and the PC12 model (FIG. 18B). Thus, the ST14A cell culture model can be used to identify compounds that are active in in vivo models of HD. In this screen, a series of structurally related compounds (Table 3 and FIG. 18A) was identified, designated as revertin-1 series (rev-1a, 1b, 1c) that selectively rescued cell death in all mutant htt-expressing cell lines (FIG. 18D). Compounds in this series were active in both the worm and PC12 HD models (FIG. 18B-C). The revertin-1 series has certain features making it an attractive candidate for lead development including different analogues that are active, a low molecular weight (˜400) making it likely that it can cross the blood brain barrier; furthermore, it is synthetically tractable.

A central feature of HD pathology is neuronal loss in the striatum and cortex that results in a fatal outcome. A number of cell culture and in vivo HD models that show enhanced cell death have been developed (Sipione S, and E Cattaneo, Mol Neurobiol 2001, 23: 21-51). A striatal neuronal cell viability assay was optimized for HTS. In this model, perturbation of cellular pathways by mutant htt enhances susceptibility to cell death by serum deprivation (Rigamonti D, et al, J Neurosci 200, 20: 3705-13). It was a simple assay performed over a short duration (3 d) and achieved a throughput of ˜5000 compounds in a single run of the assay, paving the way for larger screens. The assay resulted in 50 hits from 47,000 compounds tested (a hit rate of ˜0.1%).

Most HD cell viability screens do not differentiate between compounds that specifically target mutant htt perturbed cell death pathways and general cell death pathways (Aiken C, et al., Neurobiol Dis 2004, 16: 546-55). In this assay, both ST14A and mutant-expressing ST14A cell lines show caspase dependent cell death upon serum deprivation and were rescued by a pan-caspase inhibitor. By testing for cell death suppression in ST14A cells, compounds were differentiated that generally prevent cell death from those specific to mutant htt pathways. This is purportedly the first report of compounds that are selective for mutant-htt- enhanced cell death pathways.

The classification of active compounds based on their context-dependent rescue (Table 2) was similar to a genetic approach in which genes affecting a phenotype are likely to affect the same pathway and to affect another pathway from genes that cause a different phenotype. The identification of the mechanism of a compound in a class can help elucidate the mechanism of other compounds in the same class.

Several mechanisms have been proposed to explain HD pathology (Rangone H, et al, Pathol Biol (Paris) 2004, 52: 338-42; Ross C A Cell 2004, 118: 4-7). An important question is whether one or multiple mechanisms contribute to HD pathology. The answer to this question is essential in devising effective therapeutic strategies. Compounds were identified that prevent htt toxicity in a htt-context-dependent manner and others that are context-independent (Table 2 and FIG. 16). The simplest model to explain our results is one in which expanded polyQ in mutant htt causes toxicity by affecting multiple cellular pathways, some of these pathways are common to different htt contexts while others are unique to each htt context.

These results also raise questions about HD models used for assessing a compound's efficacy. For example, the R6/2 mouse model, in which expression of exon-1 of htt causes a rapid disease phenotype is often used to test HD therapeutics, as opposed to the YAC mice that have a FL mutant htt, but a delayed phenotype (Menalled L, and M Chesselet, Trends Pharmacol Sci 2002, 23: 32-9). In HD patient brains and transgenic mouse models, both full-length htt and fragments have been reported (Zhou H, et al. J Cell Biol 2003, 163: 109-18; DiFiglia M, et al, Science 1997, 277: 1990-3; Wellington C, et al, J Neurosci 2002, 22:7862-72). These results suggest that models that use one fragment of htt are unlikely to address all the mechanisms of htt toxicity caused by multiple contexts. Compounds that are active in multiple contexts are more likely to be effective in HD. For example, both revertin-1 and revertin-2 were effective in a context-independent manner and were effective in the worm and PC12 model.

Another finding of this study is the identification of compounds that suppress cell death by potentially enhancing the protective effects of WT htt. This raises the possibility of a new avenue of therapy by modulating the protective effects of htt for delaying HD and perhaps other neurodegenerative diseases. These compounds will serve as probes to uncover the mechanism of the protective effect of WT htt.

Results from secondary screening showed low activity of hits across different HD models. There may be variety of reasons for this, including a lack of conserved targets between yeast, worm and mammalian cells, tissue specificity of target expression (striatal cells compared to transformed neuroendocrine cells (PC12), different contexts (exon1 in PC12, yeast and N171 in worms), different levels of transgene expression (yeast and PC12 have relatively high expression) and permeability differences (yeast and worms). Despite these limitations, a few compounds that showed activity in two or more HD models were identified. This strategy of testing compounds systematically in multiple models should help in prioritizing hits identified by HTS. Results from this study also suggest that multiple distinct mechanisms are involved in mutant htt toxicity and underscore the importance of addressing multiple aberrant pathways in HD treatment.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a subject displaying multi-drug resistant cancer, comprising administering to the subject an effective amount of a compound or pharmaceutically acceptable salt thereof, wherein the compound comprises:
(a) an anti-tumor antibiotic;
(b) a protein synthesis inhibitor;
(c) a thiourea analog; or
(d) an acylated secondary amine compound,
thereby treating the subject to overcome the multi-drug resistance.

2. The method of claim 1, wherein the multi-drug resistant cancer is leukemia, breast, ovarian, and bladder cancers; small cell lung cancer; gastric cancer; sarcoma; Wilms' tumor; neuroblastoma; or thyroid cancer.

3. The method of claim 1, wherein administering comprises subcutaneous, intra-muscular, intra-peritoneal, or intravenous injection; infusion; oral, nasal, or topical delivery.

4. The method of claim 1, wherein the anti-tumor antibiotic is an anthracycline.

5. The method of claim 1, wherein the acylated secondary amine compound is a compound of Formula (I): 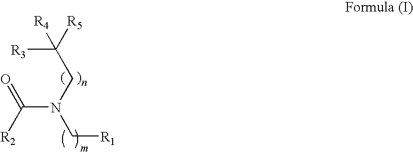
or a pharmaceutically acceptable salt thereof, wherein
R₁ is 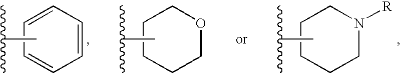
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
R₂ is C₁-C₆ alkyl, —CF₃, 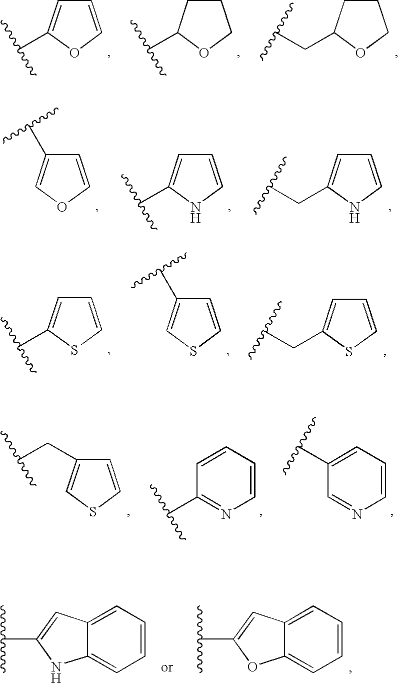
each ring optionally substituted with one or more C₁-C₆ alkyl, —O(C₁-C₆ alkyl) or —NO₂ groups;
R₃ is 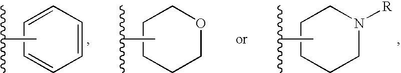
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
one of R₄ and R₅ is hydrogen and the other of R₄ and R₅ is 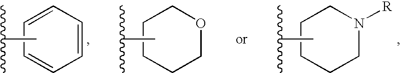
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
or R₄ and R₅ taken together with the carbon to which they are attached form 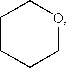
which is optionally substituted with one or more C₁-C₆ alkyl, —O(C₁-C₆ alkyl), —NH(C₁-C₆ alkyl), —N(C₁-C₆ alkyl)₂, —F or —CF₃ groups;
n is 1-3; and
m is 0-2.

6. The method of claim 1, comprising simultaneously administering a first compound or a pharmaceutically acceptable salt thereof and a second compound or a pharmaceutically acceptable salt thereof.

7. The method of claim 6, wherein the first compound or a pharmaceutically acceptable salt thereof is an anthracycline and the second compound or a pharmaceutically acceptable salt thereof is an acylated secondary amine compound.

8. The method of claim 7, wherein the acylated secondary amine compound is a compound of Formula (I): 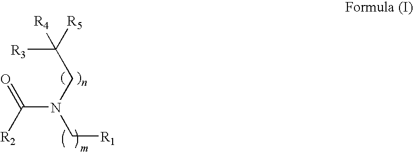
or a pharmaceutically acceptable salt thereof, wherein
R₁ is 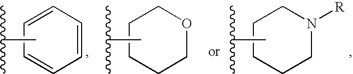
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
R₂ is C₁-C₆ alkyl, —CF₃, 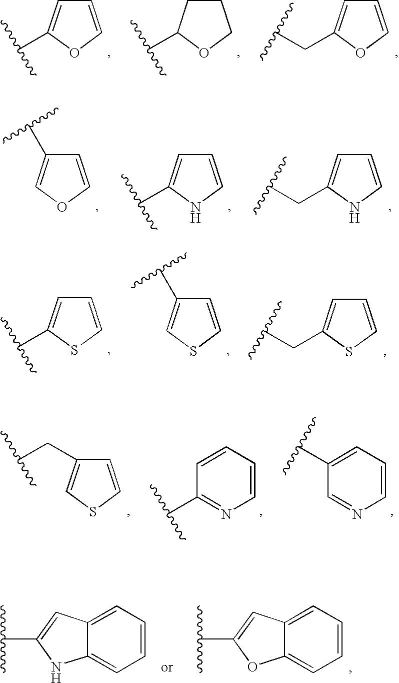
each ring optionally substituted with one or more C₁-C₆ alkyl, —O(C₁-C₆ alkyl) or —NO₂ groups;
R₃ is 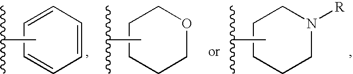
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
one of R₄ and R₅ is hydrogen and the other of R₄ and R₅ is 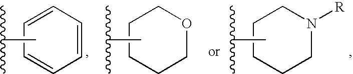
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
or R₄ and R₅ taken together with the carbon to which they are attached form 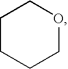
which is optionally substituted with one or more C₁-C₆ alkyl, —O(C₁-C₆ alkyl), —NH(C₁-C₆ alkyl), —N(C₁-C₆ alkyl)₂, —F or —CF₃ groups;
n is 1-3; and
m is 0-2.

9. The method of claim 7, wherein the anthracycline comprises doxorubicin, daunorubicin, nogalamycin, aclarubicin, or mitoxantrone.

10. The method of claim 7, wherein the anthracycline is doxorubicin.

11. The method of claim 1, wherein the subject is a mammal.

12. The method of claim 1, wherein the subject is human.

13. A method of treating a neurodegenerative disorder associated with polyglutamine (polyQ) expansion in a subject, comprising administering to the subject an effective amount of a compound or pharmaceutically acceptable salt thereof, wherein the compound is an acylated secondary amine compound, thereby treating a neurodegenerative disorder associated with polyglutamine (polyQ) expansion in the subject.

14. The method of claim 13, wherein the neurodegenerative disorder is Hungtinton's Disease.

15. The method of claim 13, wherein administering comprises subcutaneous, intra-muscular, intra-peritoneal, or intravenous injection; infusion; oral, nasal, or topical delivery.

16. The method of claim 13, wherein the acylated secondary amine compound is a compound of Formula (I): 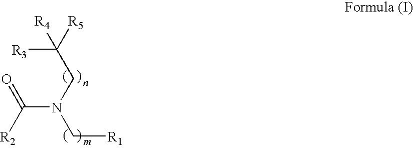
or a pharmaceutically acceptable salt thereof, wherein
R₁ is 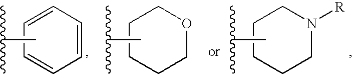
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
R₂ is C₁-C₆ alkyl, —CF₃, 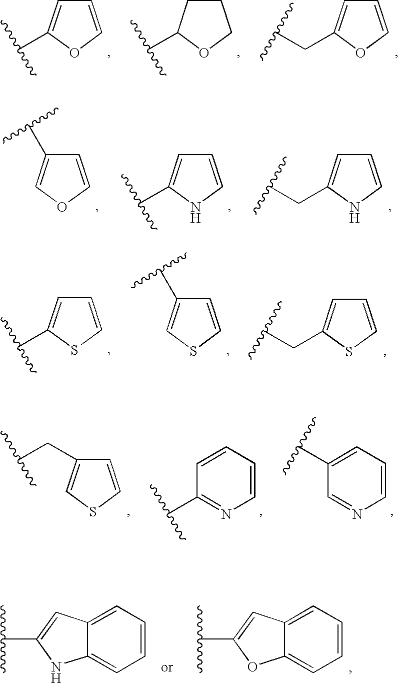
each ring optionally substituted with one or more C₁-C₆ alkyl, —O(C₁-C₆ alkyl) or —NO₂ groups;
R₃ is 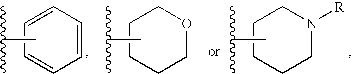
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
one of R₄ and R₅ is hydrogen and the other of R₄ and R₅ is 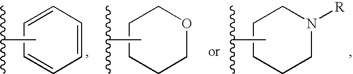
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
or R₄ and R₅ taken together with the carbon to which they are attached form 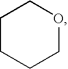
which is optionally substituted with one or more C₁-C₆ alkyl, —O(C₁-C₆ alkyl), —NH(C₁-C₆ alkyl), —N(C₁-C₆ alkyl)₂, —F or —CF₃ groups;
n is 1-3; and
m is 0-2.

17. The method of claim 16, wherein the acylated secondary amine compound is revertin-20 (Table 3).

18. The method of claim 17, wherein revertin-20 is a class IIc revertin.

19. The method of claim 18, wherein revertin-20 rescues mutant htt toxicity.

20. A method for identifying a compound that binds a motor protein, comprising
a) associating a labeled-indoxin with a motor protein to provide a labeled-indoxin/motor protein complex;
b) exposing the labeled-indoxin/motor protein complex to one or more compounds;
c) evaluating a displacement of the labeled-indoxin by the one or more compounds;
thereby identifying a compound that binds the motor protein.

21. The method of claim 20, wherein evaluating the displacement of the labeled-indoxin comprises evaluating a change in fluorescence intensity.

22. The method of claim 20, wherein the motor protein is actin-based.

23. The method of claim 22, wherein the motor protein is MyoIC.

24. The method of claim 20, wherein labeled-indoxin is conjugated with a photoprobe.

25. The method of claim 24, wherein the photoprobe is benzophenone fluorescein.

26. A compound of the Formula (II): 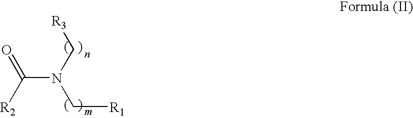
and pharmaceutically acceptable salts thereof, wherein
R₁ is 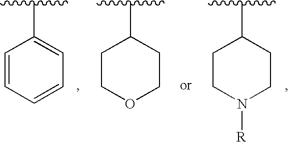
each optionally substituted with one or more C₁-C₆ alkyl, —O(C₁-C₆ alkyl), —NH(C₁-C₆ alkyl), —N(C₁-C₆ alkyl)₂, —F or —CF₃ groups;
R is hydrogen, C₁-C₆ alkyl or —CF₃;
R₂is 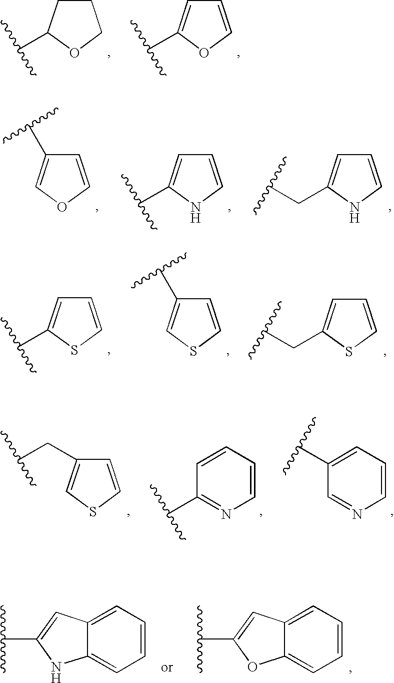
each ring optionally substituted with one or more C₁-C₆ alkyl, —O(C₁-C₆ alkyl) or —NO₂ groups;
or R₂ is 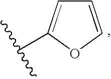
having one or more C₁-C₆ alkyl, —O(C₁-C₆ alkyl) or —NO₂ groups;
R₃ is 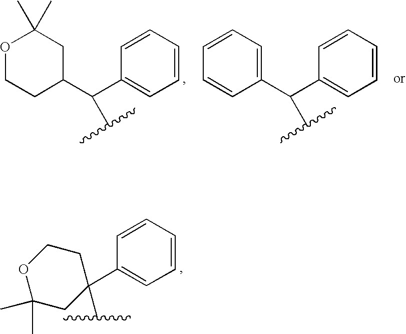
each ring optionally further substituted with one or more C₁-C₆ alkyl or —O(C₁-C₆ alkyl) groups;
n is 1-3; and
m is 0-2.

27. A sterile pharmaceutical composition comprising a compound having Formula (X) or a pharmaceutically acceptable salt thereof in an amount effective in inhibiting neuronal cell death: 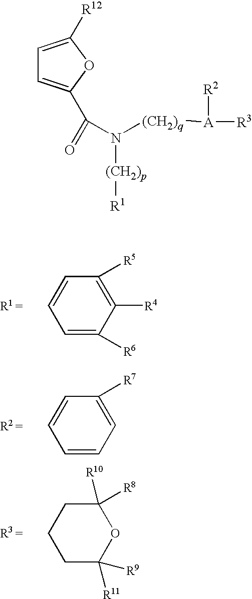
wherein A is selected from the group consisting of H and (CH);
wherein p is 0, 1, 2, or 3;
wherein q is 0, 1, 2, 3, or 4;
wherein R⁴, R⁵, and R⁶ are selected from the group consisting of H and (C₁₋₄) alkoxy;
wherein R⁷ is selected from the group consisting of H, (C₁₋₄) alkyl, and (C₁₋₄) alkoxy;
wherein R⁸, R⁹, R¹⁰ and R¹¹ are selected from the group consisting of H or (C₁₋₄) alkyl;
wherein R¹² selected from the group consisting of H, F, Cl, I, or Br.

28. The composition of claim 27, further comprising a pharmaceutical carrier.

29. A method of inhibiting neuronal cell death comprising administering, to a neuronal cell, an effective amount of a compound or pharmaceutically acceptable salt of a compound of Formula (X): 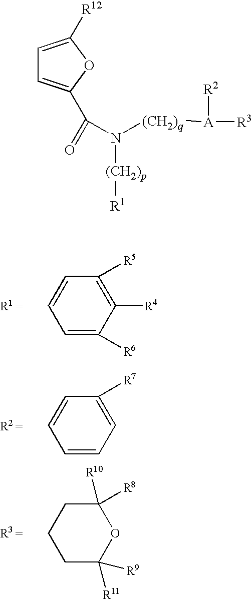
wherein A is selected from the group consisting of H and (CH);
wherein p is 0, 1, 2, or 3;
wherein q is 0, 1, 2, 3, or 4;
wherein R⁴ , R⁵, and R⁶ are selected from the group consisting of H and (C₁₋₄) alkoxy;
wherein R⁷ is selected from the group consisting of H, (C₁₋₄) alkyl, and (C₁₋₄) alkoxy;
wherein R⁸, R⁹, R¹⁰ and R¹¹ are selected from the group consisting of H or (C₁₋₄) alkyl;
wherein R¹² selected from the group consisting of H, F, Cl, I, or Br.
